SEQUENCE LISTING (2 sequences)

SEQ ID NO: 1 - protein, 163 aa, Homo sapiens
  Incyte ID NO 7514650CD1
MNSFSTSAFGPVAFSLGLLLVLPAAFPAPVPPGEDSKDVAAPHRQPLTSSERIDKQIRYILDGISALRKETCNKSNMCESSKEALAENNPNLPKMAEKDGCFQSGFNEAKNLDAITTPDPTTNASLLTKLQAQNQWLQDMTTHLILRSFKEFLQSSLRALRQM

SEQ ID NO: 2 - DNA, 962 bp, Homo sapiens
  Incyte ID NO 7514650CD1
tagaagctctatctcccctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacccgaaccttccaaagatggctgaaaaagatggatgcttccaatctggattcaatgaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattcagcaaaaaaaaaa

(19) United States
(12) Patent Application Publication
(10) Pub. No.: US 2007/0219353 A1
(43) Pub. Date: Sep. 20, 2007

(54) Immune Response Associated Proteins

(75) Inventor: Vicki S. Elliott, San Jose, CA (US)

Correspondence Address: FOLEY AND LARDNER LLP;SUITE 500, 3000 K STREET NW, WASHINGTON, DC, 20007, US

(73) Assignee: Incyte Corporation

(21) Appl. No.: 10/525,743

(22) PCT Filed: Aug. 26, 2003
(86) PCT No.: PCT/US03/26988

§ 371 Date: Sep. 27, 2005

Related U.S. Application Data
(60) Provisional application No. 60/407,566, filed on Sep. 3, 2002.
(60) Provisional application No. 60/410,178, filed on Sep. 11, 2002.
(60) Provisional application No. 60/410,571, filed on Sep. 13, 2002.
(60) Provisional application No. 60/419,906, filed on Oct. 18, 2002.
(60) Provisional application No. 60/421,445, filed on Oct. 25, 2002.

(51) Int. Cl. C07K 14/47

(52) U.S. Cl. 530/387.3; 530/387.9

(57) ABSTRACT

Various embodiments of the invention provide human immune response associated proteins (IRAP) and polynucleotides which identify and encode IRAP. Embodiments of the invention also provide expression vectors, host cells, antibodies, agonists, and antagonists. Other embodiments provide methods for diagnosing, treating, or preventing disorders associated with aberrant expression of IRAP.

### CROSS REFERENCE TO RELATED APPLICATIONS

This application is a U.S. National Stage of International Application PCT/US2003/026988, filed Aug. 26, 2003, which claims priority to U.S. Provisional Patent Application 60/407,561, filed Aug. 30, 2002, U.S. Provisional Patent Application 60/410,178, filed Sep. 11, 2002, U.S. Provisional Patent Application 60/410,571, filed Sep. 13, 2002, U.S. Provisional Patent Application 60/419,906, filed Oct. 18, 2002, and U.S. Provisional Patent Application 60/421,445, filed Oct. 25, 2002, all of which are incorporated herein by reference in their entirety.

### TECHNICAL FIELD

The invention relates to novel nucleic acids, immune response associated proteins encoded by these nucleic acids, and to the use of these nucleic acids and proteins in the diagnosis, treatment, and prevention of immune system, neurological, developmental, muscle, and cell proliferative disorders. The invention also relates to the assessment of the effects of exogenous compounds on the expression of nucleic acids and immune response associated proteins.

### BACKGROUND OF THE INVENTION

All vertebrates have developed sophisticated and complex immune systems that provide protection from viral, bacterial, fungal and parasitic infections. Included in these systems are the processes of humoral immunity, the complement cascade and the inflammatory response (See Paul, W. E. (1993) Fundamental Immunology, Raven Press, Ltd., New York N.Y. pp.1-20).

The cellular components of the immune system include six different types of leukocytes, or white blood cells: monocytes, lymphocytes, polymorphonuclear granulocytes (including neutrophils, eosinophils, and basophils) and plasma cells. Additionally, fragments of megakaryocytes, a seventh type of white blood cell in the bone marrow, occur in large numbers in the blood as platelets. Leukocytes are formed from two stem cell lineages in bone marrow. The myeloid stem cell line produces granulocytes and monocytes and the lymphoid stem cell line produces lymphocytes. Lymphoid cells travel to the thymus, spleen and lymph nodes, where they mature and differentiate into lymphocytes. Leukocytes are responsible for defending the body against invading pathogens. Neutrophils and monocytes attack invading bacteria, viruses, and other pathogens and destroy them by phagocytosis. Monocytes enter tissues and differentiate into macrophages which are extremely phagocytic. Lymphocytes and plasma cells are a part of the immune system which recognizes-specific foreign molecules and organisms and inactivates them, as well as signals other cells to attack the invaders.

Granulocytes and monocytes are formed and stored in the bone marrow until needed. Megakaryocytes are produced in bone marrow, where they fragment into platelets and are released into the bloodstream. The main function of platelets is to activate the blood clotting mechanism. Lymphocytes and plasma cells are produced in various lymphogenous organs, including the lymph nodes, spleen, thymus, and tonsils.

Both neutrophils and macrophages exhibit chemotaxis towards sites of inflammation. Tissue inflammation in response to pathogen invasion results in production of chemo-attractants for leukocytes, such as endotoxins or other bacterial products, prostaglandins, and products of leukocytes or platelets.

Basophils participate in the release of the chemicals involved in the inflammatory process. The main function of basophils is secretion of these chemicals, to such a degree that they have been referred to as ‘unicellular endocrine glands.’ A distinct aspect of basophilic secretion is that the contents of granules go directly into the extracellular environment, not into vacuoles as occurs with neutrophils, eosinophils, and monocytes. Basophils have receptors for the Fc fragment of immunoglobulin E (IgE) that are not present on other leukocytes. Crosslinking of membrane IgE with anti-IgE or other ligands triggers degranulation.

Eosinophils are bi- or multi-nucleated white blood cells which contain eosinophilic granules. Their plasma membrane is characterized by Ig receptors, particularly IgG and IgE. Generally, eosinophils are stored in the bone marrow until recruited for use at a site of inflammation or invasion. They have specific functions in parasitic infections and allergic reactions, and are thought to detoxify some of the substances released by mast cells and basophils which cause inflammation. Additionally, they phagocytize antigen-antibody complexes and further help prevent the spread of inflammation.

The mononuclear phagocyte system is comprised of precursor cells in the bone marrow, monocytes in circulation, and macrophages in tissues. Macrophages are monocytes that have left the blood stream to settle in tissue. Once monocytes have migrated into tissues, they do not re-enter the bloodstream. They increase several-fold in size and transform into macrophages that are characteristic of the tissue they have entered, surviving in tissues for several months. The mononuclear phagocyte system is capable of very fast and extensive phagocytosis. A macrophage may phagocytize over 100 bacteria, digest them and extrude residues, and then survive for many more months. Macrophages are also capable of ingesting large particles, including red blood cells and malarial parasites.

Mononuclear phagocytes are essential in defending the body against invasion by foreign pathogens, particularly intracellular microorganisms such as Mycobacterium tuberculosis, listeria, leishmania and toxoplasma. Macrophages can also control the growth of tumorous cells, via both phagocytosis and secretion of hydrolytic enzymes. Another important function of macrophages is that of processing antigens and presenting them in a biochemically modified form to lymphocytes.

The immune system responds to invading microorganisms in two major ways: antibody production and cell mediated responses. Antibodies are immunoglobulin proteins produced by B-lymphocytes which bind to specific antigens and cause inactivation or promote destruction of the antigen by other cells. Cell-mediated immune responses involve T-lymphocytes (T cells) that react with foreign antigens on the surface of infected host cells. Depending on the type of T cell, the T cell either kills the infected cell itself, or secretes signals which activate macrophages and other cells to destroy the infected cell (Paul, supra).

T-lymphocytes originate in the bone marrow or liver in fetuses. Precursor cells migrate via the blood to the thymus, where they are processed to mature into T-lymphocytes. This processing is crucial because it involves positive and negative selection of T cells for those that will react with foreign antigen and not with self molecules. After processing, T cells continuously circulate in the blood and secondary lymphoid tissues, such as lymph nodes, spleen, certain epithelium-associated tissues in the gastrointestinal tract, respiratory tract and skin. When T-lymphocytes are presented with the complementary antigen, they are stimulated to proliferate and release large numbers of activated T cells into the lymph system and the blood system. These activated T cells can survive and circulate for several days. At the same time, T memory cells are created, which remain in the lymphoid tissue for months or years. Upon subsequent exposure to that specific antigen, these memory cells will respond more rapidly and with a stronger response than induced by the original antigen. This creates an ‘immunological memory’ that can provide immunity for years.

There are two major types of T cells: cytotoxic T cells destroy infected host cells, and helper T cells activate other white blood cells via chemical signals. One class of helper cell, T_{H}1, activates macrophages to destroy ingested microorganisms, while another, T_{H}2, stimulates the production of antibodies by B cells.

Cytotoxic T cells directly attack the infected target cell. Receptors on the surface of T cells bind to antigen presented by MHC molecules on the surface of the infected cell. Once activated by binding to antigen, T cells secrete γ-interferon, a signal molecule that induces the expression of genes necessary for presenting viral (or other) antigens to cytotoxic T cells. Cytotoxic T cells kill the infected cell by stimulating programmed cell death.

Helper T cells constitute up to 75% of the total T cell population. They regulate the immune functions by producing a variety of lymphokines that act on other cells in the immune system and on bone marrow. Among these lymphokines are interleukins 2 through 6, granulocyte-monocyte colony stimulating factor, and γ-interferon.

Helper T cells are required for most B cells to respond to antigen. When an activated helper cell contacts a B cell, its centrosome and Golgi apparatus become oriented toward the B cell, aiding the directing of signal molecules, such as a transmembrane-bound protein called CD40 ligand, onto the B cell surface to interact with the CD40 transmembrane protein. Secreted signals also help B cells to proliferate and mature and, in some cases, to switch the class of antibody being produced.

B-lymphocytes (B cells) produce antibodies which react with specific antigenic proteins presented by pathogens. Once activated, B cells become filled with extensive rough endoplasmic reticulum and are known as plasma cells. As with T cells, interaction of B cells with antigen stimulates proliferation of only those B cells which produce antibody specific to that antigen. There are five classes of antibodies, known as immunoglobulins, which together comprise about 20% of total plasma protein. Each class mediates a characteristic biological response after antigen binding. Upon activation by specific antigen B cells switch from making the membrane-bound antibody to the secreted form of that antibody.

Antibodies, or immunoglobulins, are the founding members of the immunoglobulin (Ig) superfamily and the central components of the humoral immune response. Antibodies are either expressed on the surface of B cells or secreted by B cells into the circulation. Antibodies bind and neutralize blood-borne foreign antigens. The prototypical antibody is a tetramer consisting of two identical heavy polypeptide chains (H-chains) and two identical light polypeptide chains (L-chains) interlinked by disulfide bonds. This arrangement confers the characteristic Y-shape to antibody molecules. Antibodies are classified based on their H-chain composition. The five antibody classes, IgA, IgD, IgE, IgG and IgM, are defined by the α, δ, ε, γ, and μ H-chain types. There are two types of L-chains, κ and λ, either of which may associate as a pair with any H-chain pair. IgG, the most common class of antibody found in the circulation, is tetrameric, while the other classes of antibodies are generally variants or multimers of this basic structure.

H-chains and L-chains each contain an N-terminal variable region and a C-terminal constant region. The constant region consists of about 110 amino acids in L-chains and about 330 or 440 amino acids in H-chains. The amino acid sequence of the constant region is nearly identical among H- or L-chains of a particular class. The variable region consists of about 110 amino acids in both H- and L-chains. However, the amino acid sequence of the variable region differs among H- or L-chains of a particular class. Within each H- or L-chain variable region are three hypervariable regions of extensive sequence diversity, each consisting of about 5 to 10 amino acids. In the antibody molecule, the H- and L-chain hypervariable regions come together to form the antigen recognition site. (Reviewed in Alberts, B. et al. (1994) Molecular Biology of the Cell, Garland Publishing, New York, N.Y., pp.1206-1213 and 1216-1217.)

The immune system is capable of recognizing and responding to any foreign molecule that enters the body. Therefore, the immune system must be armed with a full repertoire of antibodies against all potential antigens. Such antibody diversity is generated by somatic rearrangement of gene segments encoding variable and constant regions. These gene segments are joined together by site-specific recombination which occurs between highly conserved DNA sequences that flank each gene segment. Because there are hundreds of different gene segments, millions of unique genes can be generated combinatorially. In addition, imprecise joining of these segments and an unusually high rate of somatic mutation within these segments further contribute to the generation of a diverse antibody population.

Both H-chains and L-chains contain repeated Ig domains. For example, a typical H-chain contains four Ig domains, three of which occur within the constant region and one of which occurs within the variable region and contributes to the formation of the antigen recognition site. Likewise, a typical L-chain contains two Ig domains, one of which occurs within the constant region and one of which occurs within the variable region. In addition, H chains such as μ have been shown to associate with other polypeptides during differentiation of the B-cell.

Antibodies can be described in terms of their two main functional domains. Antigen recognition is mediated by the Fab (antigen binding fragment) region of the antibody, while effector functions are mediated by the Fc (crystallizable fragment) region. Binding of antibody to an antigen, such as a bacterium, triggers the destruction of the antigen by phagocytic white blood cells such as macrophages and neutrophils. These cells express surface receptors that specifically bind to the antibody Fc region and allow the phagocytic cells to engulf, ingest, and degrade the antibody-bound antigen. The Fc receptors expressed by phagocytic cells are single-pass transmembrane glycoproteins of about 300 to 400 amino acids (Sears, D. W. et al. (1990) J. Immunol. 144:371-378). The extracellular portion of the Fc receptor typically contains two or three Ig domains.

Diseases which cause over- or under-abundance of any one type of leukocyte usually result in the entire immune defense system becoming involved. The most notorious autoimmune disease is AIDS (Acquired Immunodeficiency Syndrome). This disease depletes the number of helper T cells and leaves the patient susceptible to infection by microorganisms and parasites.

Another widespread medical condition attributable to the immune system is that of allergic reactions to certain antigens. Delayed reaction allergy is experienced by many genetically normal people. In the case of atopic allergies, there is a genetic origin, such that large quantities of IgE antibodies are produced. IgEs have a strong tendency to attach to mast cells and basophils, up to half million each (IgE/mast) which then rupture and release histamine, leukotrienes, eosinophil chemotactic substance, protease, neutrophil chemotactic substance, heparin, and platelet activation factors. Tissues can respond in a number of ways to these substances resulting in what are commonly known as allergic reactions: hay fever, asthma, anaphylaxis, and urticaria (hives).

Leukemias are an excess production of white blood cells, to the point where a major portion of the body's metabolic resources are directed solely at proliferation of white blood cells, leaving other tissues to starve. With lymphogenous leukemias, cancerous lymphogenous cells spread from a lymph node to other body parts. Excess T- and B-lymphocytes are produced. In myelogenous leukemias, cancerous young myelogenous cells spread from the bone marrow to other organs, especially the spleen, liver, lymph nodes and other highly vascularized regions. Usually, the extra leukemic cells released are immature, incapable of function, and undifferentiated. Occasionally, partially differentiated cells are produced, leading to classification of the disease as neutrophilic leukemia, eosinophilic leukemia, basophilic leukemia, or monocytic leukemia. Leukemias may be caused by exposure to environmental factors such as radiation or toxic chemicals or by genetic aberration.

Leukopenia or agranulocytosis occurs when the bone marrow stops producing white blood cells. This leaves the body unprotected against foreign microorganisms, including those which normally inhabit skin, mucous membranes, and gastrointestinal tract. If all white blood cell production stops completely, infection will occur within two days and death may follow only 1 to 4 days later. Acute leukopenia can be caused by exposure to radiation or chemicals containing benzene. Occasionally, drugs such as chloramphenicol and thiouracil can suppress blood cell production by the bone marrow and initiate the onset of agranulocytosis. In cases of monoblastic leukemia, primitive monocytes in blood and bone marrow do not mature. Clinical symptoms reflect this abnormality: high lysozyme levels in blood serum, renal tubular dysfunction, and high fevers.

Impaired phagocytosis occurs in several diseases, including monocytic leukemia, systemic lupus, and granulomatous disease. In such a situation, macrophages can phagocytize normally, but the enveloped organism is not killed. There is a defect in the plasma membrane enzyme which converts oxygen to lethally reactive forms. This results in abscess formation in liver, lungs, spleen, lymph nodes, and beneath the skin.

Eosinophilia is an excess of eosinophils commonly observed in patients with allergies (hay fever, asthma), allergic reactions to drugs, rheumatoid arthritis, and cancers (Hodgkins disease, lung, and liver cancer). The mechanism for elevated levels of eosinophils in these diseases is unknown. (Isselbacher, K. J. et al. (1994) Harrison's Principles of Internal Medicine, McGraw-Hill, Inc., New York, N.Y.).

Host defense is further augmented by the complement system. The complement system serves as an effector system and is involved in infectious agent recognition. It can function as an independent immune network or in conjunction with other humoral immune responses. The complement system is comprised of numerous plasma and membrane proteins that act in a cascade of reaction sequences whereby one component activates the next. The result is a rapid and amplified response to infection through either an inflammatory response or increased phagocytosis.

The complement system has more than 30 protein components which can be divided into functional groupings including modified serine proteases, membrane-binding proteins, and regulators of complement activation. Activation occurs through two different pathways, the classical and the alternative. Both pathways serve to destroy infectious agents through distinct triggering mechanisms that eventually merge with the involvement of the component C3.

The classical pathway requires antibody binding to infectious agent antigens. The antibodies serve to define the target and initiate the complement system cascade, culminating in the destruction of the infectious agent. In this pathway, since the antibody guides initiation of the process, the complement system can be seen as an effector arm of the humoral immune system.

The alternative pathway of the complement system does not require the presence of pre-existing antibodies for targeting infectious agent destruction. Rather, this pathway, through low levels of an activated component, remains constantly primed and provides surveillance in the non-immune host to enable targeting and destruction of infectious agents. In this case foreign material triggers the cascade, thereby facilitating phagocytosis or lysis (Paul, supra pp.918-919).

Another important component of host defense is the process of inflamation. Inflammatory responses are divided into four categories on the basis of pathology and include allergic inflammation, cytotoxic antibody mediated inflammation, immune complex mediated inflammation, and monocyte mediated inflammation. Inflammation manifests as a combination of each of these forms with one predominating.

Allergic acute inflamation is observed in individuals wherein specific antigens stimulate IgE antibody production. Mast cells and basophils are subsequently activated by the attachment of antigen-IgE complexes, resulting in the release of cytoplasmic granule contents such as histamine. The products of activated mast cells can increase vascular permeability and constrict the smooth muscle of breathing passages, resulting in anaphylaxis or asthma.

Acute inflamation is also mediated by cytotoxic antibodies and can result in the destruction of tissue through the binding of complement-fixing antibodies to cells. In this case the antibodies responsible are of the IgG or IgM types and resultant clinical disorders including autoimmune hemolytic anemia and thrombocytopenia as associated with systemic lupus erythematosis.

Immune complex mediated acute inflammation involves the IgG or IgM antibody types which combine with antigen to activate the complement cascade. When such immune complexes bind to neutrophils and macrophages they activate the respiratory burst to form protein and vessel damaging agents such as hydrogen peroxide, hydroxyl radical, hypochlorous acid, and chloramines. Clinical manifestations include rheumatoid arthritis and systemic lupus erythematosus.

In chronic inflammation or delayed-type hypersensitivity, macrophages are activated and process antigen for presentation to T cells that subsequently produce lymphokines and monokines. This type of inflammatory response is likely important for defense against intracellular parasites and certain viruses. Clinical associations include granulomatous disease, tuberculosis, leprosy, and sarcoidosis (Paul, supra pp.1017-1018).

Most cell surface and soluble molecules that mediate functions such as recognition, adhesion or binding have evolved from a common evolutionary precursor (i.e., these proteins have structural homology). A number of molecules outside the immune system that have similar functions are also derived from this same evolutionary precursor. These molecules are classified as members of the immunoglobulin (Ig) superfamily. The criteria for a protein to be a member of the Ig superfamily is to have one or more Ig domains, which are regions of 70-110 amino acid residues in length homologous to either Ig variable-like (V) or Ig constant-like (C) domains. Members of the Ig superfamily include antibodies (Ab), T cell receptors (TCRs), class I and II major histocompatibility (MHC) proteins, CD2, CD3, CD4, CD8, poly-Ig receptors, Fc receptors, neural cell-adhesion molecule (NCAM) and platelet-derived growth factor receptor (PDGFR).

Ig domains (V and C) are regions of conserved amino acid residues that give a polypeptide a globular tertiary structure called an immunoglobulin (or antibody) fold, which consists of two approximately parallel layers of β-sheets. Conserved cysteine residues form an intrachain disulfide-bonded loop, 55-75 amino acid residues in length, which connects the two layers of the β-sheets. Each β-sheet has three or four anti-parallel β-strands of 5-10 amino acid residues. Hydrophobic and hydrophilic interactions of amino acid residues within the β-strands stabilize the Ig fold (hydrophobic on inward facing amino acid residues and hydrophilic on the amino acid residues in the outward facing portion of the strands). A V domain consists of a longer polypeptide than a C domain, with an additional pair of β-strands in the Ig fold.

A consistent feature of Ig superfamily genes is that each sequence of an Ig domain is encoded by a single exon. It is possible that the superfamily evolved from a gene coding for a single Ig domain involved in mediating cell-cell interactions. New members of the superfamily then arose by exon and gene duplications. Modern Ig superfamily proteins contain different numbers of V and/or C domains. Another evolutionary feature of this superfamily is the ability to undergo DNA rearrangements, a unique feature retained by the antigen receptor members of the family.

Many members of the Ig superfamily are integral plasma membrane proteins with extracellular Ig domains. The hydrophobic amino acid residues of their transmembrane domains and their cytoplasmic tails are very diverse, with little or no homology among Ig family members or to known signal-transducing structures. There are exceptions to this general superfamily description. For example, the cytoplasmic tail of PDGFR has tyrosine kinase activity. In addition Thy-1 is a glycoprotein found on thymocytes and T cells. This protein has no cytoplasmic tail, but is instead attached to the plasma membrane by a covalent glycophosphatidylinositol linkage.

Another common feature of many Ig superfamily proteins is the interactions between Ig domains which are essential for the function of these molecules. Interactions between Ig domains of a multimeric protein can be either homophilic or heterophilic (i.e., between the same or different Ig domains). Antibodies are multimeric proteins which have both homophilic and heterophilic interactions between Ig domains. Pairing of constant regions of heavy chains forms the Fc region of an antibody and pairing of variable regions of light and heavy chains form the antigen binding site of an antibody. Heterophilic interactions also occur between Ig domains of different molecules. These interactions provide adhesion between cells for significant cell-cell interactions in the immune system and in the developing and mature nervous system. (Reviewed in Abbas, A. K. et al. (1991) Cellular and Molecular Immunology, W. B. Saunders Company, Philadelphia, Pa., pp.142-145.)

Sushi domains, also known as complement control protein (CCP) modules, or short consensus repeats (SCR), are found in a wide variety of complement and adhesion proteins. CD21 (also called C3d receptor, CR2, Epstein Barr virus receptor or EBV-R) is the receptor for EBV and for C3d, C3dg and iC3b. Complement components may activate B cells through CD21. CD21 is part of a large signal-transduction complex that also involves CD19, CD81, and Leul13. Some of the proteins in this group are responsible for the molecular basis of the blood group antigens, surface markers on the outside of the red blood cell membrane. Most of these markers are proteins, but some are carbohydrates attached to lipids or proteins (for a review see Reid, M. E. and C. Lomas-Francis (1977) The Blood Group Antigen FactsBook Academic Press, San Diego, Calif.). Complement decay-accelerating factor (Antigen CD55) belongs to the Cromer blood group system and is associated with Cr(a), Dr(a), Es(a), Tc(a/b/c), Wd(a), WES(a/b), IFC and UMC antigens. Complement receptor type 1 (C3b/C4b receptor) (Antigen CD35) belongs to the Knops blood group system and is associated with Kn(a/b), McC(a), Sl(a) and Yk(a) antigens.

Human leukocyte-specific transcript 1 (LST1) is a small protein that modulates immune responses and cellular morphogenesis. LST1 is expressed at high levels in dendritic cells. A DNA-binding site and interaction of multiple regulatory elements may be involved in mediating the expression of the various forms of LST1 mRNA (Yu, X. and Weissman, S. M. (2000) J. Biol. Chem. 275:34597-34608).

Spalpha is a member of the scavenger receptor cysteine-rich (SRCR) family of proteins. Spalpha is expressed only in lymphoid tissues, where it is implicated in monocyte activity (Gebe, J. A. (1997) J. Biol. Chem. 272:6151-6158). Expression profiling

Microarrays are analytical tools used in bioanalysis. A microarray has a plurality of molecules spatially distributed over, and stably associated with, the surface of a solid support. Microarrays of polypeptides, polynucleotides, and/or antibodies have been developed and find use in a variety of applications, such as gene sequencing, monitoring gene expression, gene mapping, bacterial identification, drug discovery, and combinatorial chemistry.

One area in particular in which microarrays find use is in gene expression analysis. Array technology can provide a simple way to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for identifying genes that are tissue specific, are affected by a substance being tested in a toxicology assay, are part of a signaling cascade, carry out housekeeping functions, or are specifically related to a particular genetic predisposition, condition, disease, or disorder.

Breast Cancer

More than 180,000 new cases of breast cancer are diagnosed each year, and the mortality rate for breast cancer approaches 10% of all deaths in females between the ages of 45-54 (Gish, K. (1999) AWIS Magazine 28:7-10). However the survival rate based on early diagnosis of localized breast cancer is extremely high (97%), compared with the advanced stage of the disease in which the tumor has spread beyond the breast (22%). Current procedures for clinical breast examination are lacking in sensitivity and specificity, and efforts are underway to develop comprehensive gene expression profiles for breast cancer that may be used in conjunction with conventional screening methods to improve diagnosis and prognosis of this disease (Perou, C. M. et al. (2000) Nature 406:747-752).

Mutations in two genes, BRCA1 and BRCA2, are known to greatly predispose a woman to breast cancer and may be passed on from parents to children (Gish, supra). However, this type of hereditary breast cancer accounts for only about 5% to 9% of breast cancers, while the vast majority of breast cancer is due to non-inherited mutations that occur in breast epithelial cells.

The relationship between expression of epidermal growth factor (EGF) and its receptor, EGFR, to human mammary carcinoma has been particularly well studied. (See Khazaie, K. et al. (1993) Cancer and Metastasis Rev. 12:255-274, and references cited therein for a review of this area.) Overexpression of EGFR, particularly coupled with down-regulation of the estrogen receptor, is a marker of poor prognosis in breast cancer patients. In addition, EGFR expression in breast tumor metastases is frequently elevated relative to the primary tumor, suggesting that EGFR is involved in tumor progression and metastasis. This is supported by accumulating evidence that EGF has effects on cell functions related to metastatic potential, such as cell motility, chemotaxis, secretion and differentiation. Changes in expression of other members of the erbB receptor family, of which EGFR is one, have also been implicated in breast cancer. The abundance of erbB receptors, such as HER-2/neu, HER-3, and HER-4, and their ligands in breast cancer points to their functional importance in the pathogenesis of the disease, and may therefore provide targets for therapy of the disease (Bacus, S. S. et al. (1994) Am. J. Clin. Pathol. 102:S13-S24). Other known markers of breast cancer include a human secreted frizzled protein mRNA that is downregulated in breast tumors; the matrix G1a protein which is overexpressed in human breast carcinoma cells; Drg1 or RTP, a gene whose expression is diminished in colon, breast, and prostate tumors; maspin, a tumor suppressor gene downregulated in invasive breast carcinomas; and CaN19, a member of the S100 protein family, all of which are down-regulated in mammary carcinoma cells relative to normal mammary epithelial cells (Zhou, Z. et al. (1998) Int. J. Cancer 78:95-99; Chen, L. et al. (1990) Oncogene 5:1391-1395; Ulrix, W. et al (1999) FEBS Lett 455:23-26; Sager, R. et al. (1996) Curr. Top. Microbiol. Immunol. 213:51-64; and Lee, S. W. et al. (1992) Proc. Natl. Acad. Sci. USA 89:2504-2508).

Cell lines derived from human mammary epithelial cells at various stages of breast cancer provide a useful model to study the process of malignant transformation and tumor progression as it has been shown that these cell lines retain many of the properties of their parental tumors for lengthy culture periods (Wistuba, I. I. et al. (1998) Clin. Cancer Res. 4:2931-2938). Such a model is particularly useful for comparing phenotypic and molecular characteristics of human mammary epithelial cells at various stages of malignant transformation.

Colon Cancer

Colorectal cancer is the second leading cause of cancer deaths in the United States, and is thought to be a disease of aging since 90% of the total cases occur in individuals over the age of 55. A widely accepted hypothesis is that several mutations must accumulate over time in an individual who develops the disease.

While soft tissue sarcomas are relatively rare, more than 50% of new patients diagnosed with the disease will die from it. The molecular pathways leading to the development of sarcomas are relatively unknown, due to the rarity of the disease and variation in pathology. Colon cancer evolves through a multi-step process whereby pre-malignant colonocytes undergo a relatively defined sequence of events leading to tumor formation. Several factors participate in the process of tumor progression and malignant transformation including genetic factors, mutations, and selection.

To understand the nature of gene alterations in colorectal cancer, a number of studies have focused on the inherited syndromes. The first, Familial Adenomatous Polyposis (FAP), is caused by mutations in the Adenomatous Polyposis Coli gene (APC), resulting in truncated or inactive forms of the protein. This tumor suppressor gene has been mapped to chromosome 5q. The second known inherited syndrome is hereditary nonpolyposis colorectal cancer (HNPCC), which is caused by mutations in mismatch repair genes.

Although hereditary colon cancer syndromes occur in a small percentage of the population, and most colorectal cancers are considered sporadic, knowledge from studies of the hereditary syndromes can be applied broadly. For instance, somatic mutations in APC occur in at least 80% of sporadic colon tumors. APC mutations are thought to be the initiating event in disease progression. Other mutations occur subsequently. Approximately 50% of colorectal cancers contain activating mutations in ras, while 85% contain inactivating mutations in p53. Changes in all of these genes lead to gene expression changes in colon cancer. Less is understood about downstream targets of these mutations and the role they may play in cancer development and progression.

Lung Cancer

Lung cancer is the leading cause of cancer death for men and the second leading cause of cancer death for women in the U.S. Lung cancers are divided into four histopathologically distinct groups. Three groups (squamous cell carcinoma, adenocarcinoma, and large cell carcinoma) are classified as non-small cell lung cancers (NSCLCs). The fourth group of cancers is referred to as small cell lung cancer (SCLC). Deletions on chromosome 3 are common in this disease and are thought to indicate the presence of a tumor suppressor gene in this region. Activating mutations in K-ras are commonly found in lung cancer and are the basis of one of the mouse models for the disease.

The potential application of gene expression profiling is particularly relevant to improving diagnosis, prognosis, and treatment of cancer, such as lung cancer. Lung cancer is the leading cause of cancer death in the United States, affecting more than 100,000 men and 50,000 women each year. Nearly 90% of the patients diagnosed with lung cancer are cigarette smokers. Tobacco smoke contains thousands of noxious substances that induce carcinogen metabolizing enzymes and covalent DNA adduct formation in the exposed bronchial epithelium. In nearly 80% of patients diagnosed with lung cancer, metastasis has already occurred. Most commonly lung cancers metastasize to pleura, brain, bone, pericardium, and liver. The decision to treat with surgery, radiation therapy, or chemotherapy is made on the basis of tumor histology, response to growth factors or hormones, and sensitivity to inhibitors or drugs. With current treatments, most patients die within one year of diagnosis. Earlier diagnosis and a systematic approach to identification, staging, and treatment of lung cancer could positively affect patient outcome.

Lung cancers progress through a series of morphologically distinct stages from hyperplasia to invasive carcinoma. Malignant lung cancers are divided into two groups comprising four histopathological classes. The Non Small Cell Lung Carcinoma (NSCLC) group includes squamous cell carcinomas, adenocarcinomas, and large cell carcinomas and accounts for about 70% of all lung cancer cases. Adenocarcinomas typically arise in the peripheral airways and often form mucin secreting glands. Squamous cell carcinomas typically arise in proximal airways. The histogenesis of squamous cell carcinomas may be related to chronic inflammation and injury to the bronchial epithelium, leading to squamous metaplasia. The Small Cell Lung Carcinoma (SCLC) group accounts for about 20% of lung cancer cases. SCLCs typically arise in proximal airways and exhibit a number of paraneoplastic syndromes including inappropriate production of adrenocorticotropin and anti-diuretic hormone.

Lung cancer cells accumulate numerous genetic lesions, many of which are associated with cytologically visible chromosomal aberrations. The high frequency of chromosomal deletions associated with lung cancer may reflect the role of multiple tumor suppressor loci in the etiology of this disease. Deletion of the short arm of chromosome 3 is found in over 90% of cases and represents one of the earliest genetic lesions leading to lung cancer. Deletions at chromosome arms 9p and 17p are also common. Other frequently observed genetic lesions include overexpression of telomerase, activation of oncogenes such as K-ras and c-myc, and inactivation of tumor suppressor genes such as RB, p53 and CDKN2.

Genes differentially regulated in lung cancer have been identified by a variety of methods. Using mRNA differential display technology, Manda et al. (1999; Genomics 51:5-14) identified five genes differentially expressed in lung cancer cell lines compared to normal bronchial epithelial cells. Among the known genes, pulmonary surfactant apoprotein A and alpha 2 macroglobulin were down regulated whereas nm23H1 was upregulated. Petersen et al. (2000; Int J. Cancer, 86:512-517) used suppression subtractive hybridization to identify 552 clones differentially expressed in lung tumor derived cell lines, 205 of which represented known genes. Among the known genes, thrombospondin-1, fibronectin, intercellular adhesion molecule 1, and cytokeratins 6 and 18 were previously observed to be differentially expressed in lung cancers. Wang et al. (2000; Oncogene 19:1519-1528) used a combination of microarray analysis and subtractive hybridization to identify 17 genes differentially overexpresssed in squamous cell carcinoma compared with normal lung epithelium. Among the known genes they identified were keratin isoform 6, KOC, SPRC, IGFb2, connexin 26, plakofillin 1 and cytokeratin 13.

Immune Responses

Human peripheral blood mononuclear cells (PBMCs) represent the major cellular components of the immune system. PBMCs contain about 12% B lymphocytes, 25% CD4+and 15% CD8+lymphocytes, 20% NK cells, 25% monocytes, and 3% various cells that include dendritic cells and progenitor cells. The proportions, as well as the biology of these cellular components tend to vary slightly between healthy individuals, depending on factors such as age, gender, past medical history, and genetic background.

Interleukin 1 beta (IL-1β) is a cytokine associated with acute inflammatory responses and is generally considered the prototypical pro-inflammatory cytokine. However, IL-1β functions are not limited to the inflammatory response since this molecule is involved in processes such as fever induction, metabolic regulation, and bone remodeling. Both cells of the immune system (monocytes, dendritic cells, NK cells, platelets, and neutrophils) and somatic cells (osteoblasts, neurons, Schwann's cells, oligodendrocytes, and adrenal cortical cells) can produce IL-1β. IL-1β has been shown to induce its own production in monocytes; induce the production of adhesion molecules and chemokines in endothelial cells; and in conjunction with IL-12, induce interferon-γ production by NK cells. IL-1β is produced as a single chain pro-molecule that needs to be cleaved by a specialized protease, IL-1β converting enzyme (ICE), to acquire its function.

Leptin is a protein product of the mouse obesity gene. Mice with mutations in the obesity gene that block the synthesis of leptin tend to be obese and diabetic and exhibit reduced activity, metabolism, and body temperature. Human leptin shares approximately 84% sequence identity with the mouse protein. Human leptin cDNA encodes a 167 amino acid residue protein with a 21 amino acid residue signal sequence that is cleaved to yield the 146 amino acid residue mature protein. The expression of leptin mRNA is restricted to adipose tissue. A high-affinity receptor for leptin (OB-R) with homology to gp130 and the G-CSF receptor has recently been cloned. OB-R mRNA is expressed in the choroid plexus and in the hypothalamus. OB-R is also an isoform of B219, a sequence that is expressed in at least four isoforms in very primitive hematopoietic cell populations and in a variety of lymphohematopoietic cell lines. The possible roles of leptin in body weight regulation, hematopoiesis, and reproduction are being investigated.

Leukemia inhibitory factor (LIF) was initially identified as a factor that inhibits the proliferation and induces the differentiation to macrophages of the murine myeloid leukemic cell line M1. Subsequent to its purification and molecular cloning, LIF was recognized to be a pleiotropic factor with multiple effects on both hematopoietic and non-hematopoietic cells. LIF has overlapping biological functions with OSM, IL-6, IL-11, and CNTF. All these cytokines use gp130 as a component in their signal transducing receptor complexes. Human LIF cDNA encodes a 202 amino acid residue polypeptide with a 22 amino acid residue signal peptide that is cleaved to yield a 180 amino acid residue mature human LIF.

Tumor necrosis factor alpha (TNF-α), also called cachectin, is produced by neutrophils, activated lymphocytes, macrophages, NK cells, LAK cells, astrocytes, endothelial cells, smooth muscle cells, and some transformed cells. TNF-α occurs as a secreted, soluble form and as a membrane-anchored form, both of which are biologically active. Two types of receptors for TNF-α have been described and virtually all cell types studied show the presence of one or both of these receptor types. TNF-α and TNF-β are extremely pleiotropic factors due to the ubiquity of their receptors, to their ability to activate multiple signal transduction pathways, and to their ability to induce or suppress the expression of a wide number of genes. TNF-α and TNF-β play a critical role in mediation of the inflammatory response and in mediation of resistance to infections and tumor growth.

Monocytes are involved in the initiation and maintenance of inflammatory immune responses. The outer membrane of gram-negative bacteria expresses lipopolysaccharide (LPS) complexes called endotoxins. Toxicity is associated with the lipid component (Lipid A) of LPS, and immunogenicity is associated with the polysaccharide components of LPS. LPS elicits a variety of inflammatory responses, and because it activates complement by the alternative (properdin) pathway, it is often part of the pathology of gram-negative bacterial infections. For the most part, endotoxins remain associated with the cell wall until the bacteria disintegrate. LPS released into the bloodstream by lysing gram-negative bacteria is first bound by certain plasma proteins identified as LPS-binding proteins. The LPS-binding protein complex interacts with CD14 receptors on monocytes, macrophages, B cells, and other types of receptors on endothelial cells. Activation of human B cells with LPS results in mitogenesis as well as immunoglobulin synthesis. In monocytes and macrophages three types of events are triggered during their interaction with LPS: 1) production of cytokines, including IL-1, IL-6, IL-8, TNF-α, and platelet-activating factor, which stimulate production of prostaglandins and leukotrienes that mediate inflammation and septic shock; 2) activation of the complement cascade; and 3) activation of the coagulation cascade.

Staphylococcal exotoxins such as staphylococcal exotoxin B (SEB) specifically activate human T cells, expressing an appropriate TCR-Vbeta chain. Although polyclonal in nature, T cells activated by Staphylococcal exotoxins require antigen presenting cells (APCs) to present the exotoxin molecules to the T cells and deliver the costimulatory signals required for optimum T cell activation. Although Staphylococcal exotoxins must be presented to T cells by APCs, these molecules are not required to be processed by APC. Indeed, Staphylococcal exotoxins directly bind to a non-polymorphic portion of the human MHC class II molecules, bypassing the need for capture, cleavage, and binding of the peptides to the polymorphic antigenic groove of the MHC class II molecules.

Cyclosporin A (CsA) has been a drug of choice for the last 15 years for inmmunosuppression in organ transplants, GvHD in bone marrow transplants, and in some other inflammatory conditions. CsA forms complexes with cyclophilin molecules that inhibit the cytoplasmic phosphatase calcineurin. Inhibition of the phosphatase activity of calcineurin prevents the downstream induction of transcription of genes such as IL-2, IL-3, and IFN-γ. In the absence of these terminal mediators, T cell activation is aborted and the development of the immune response is dramatically impaired.

Obesity

The most important function of adipose tissue is its ability to store and release fat during periods of feeding and fasting. White adipose tissue is the major energy reserve in periods of excess energy use. Its primary purpose is mobilization during energy deprivation. Understanding how various molecules regulate adiposity and energy balance in physiological and pathophysiological situations may lead to the development of novel therapeutics for human obesity. Adipose tissue is also one of the important target tissues for insulin. Adipogenesis and insulin resistance in type II diabetes are linked and present intriguing relations. Most patients with type II diabetes are obese and obesity in turn causes insulin resistance.

The majority of research in adipocyte biology to date has been done using transformed mouse preadipocyte cell lines. The culture condition which stimulates mouse preadipocyte differentiation is different from that for inducing human primary preadipocyte differentiation. In addition, primary cells are diploid and may therefore reflect the in vivo context better than aneuploid cell lines. Understanding the gene expression profile during adipogenesis in humans will lead to an understanding of the fundamental mechanism of adiposity regulation. Furthermore, through comparing the gene expression profiles of adipogenesis between donor with normal weight and donor with obesity, identification of crucial genes, potential drug targets for obesity and type II diabetes, will be possible.

Thiazolidinediones (TZDs) act as agonists for the peroxisome-proliferator-activated receptor gamma (PPARγ), a member of the nuclear hormone receptor superfamily. TZDs reduce hyperglycemia, hyperinsulinemia, and hypertension, in part by promoting glucose metabolism and inhibiting gluconeogenesis. Roles for PPARγ and its agonists have been demonstrated in a wide range of pathological conditions including diabetes, obesity, hypertension, atherosclerosis, polycystic ovarian syndrome, and cancers such as breast, prostate, liposarcoma, and colon cancer.

The mechanism by which TZDs and other PPARγ agonists enhance insulin sensitivity is not fully understood, but may involve the ability of PPARγ to promote adipogenesis. When ectopically expressed in cultured preadipocytes, PPARγ is a potent inducer of adipocyte differentiation. TZDs, in combination with insulin and other factors, can also enhance differentiation of human preadipocytes in culture (Adams, M. et al. (1997) J. Clin. Invest. 100:3149-3153). The relative potency of different TZDs in promoting adipogenesis in vitro is proportional to both their insulin sensitizing effects in vivo, and their ability to bind and activate PPARγ in vitro. Interestingly, adipocytes derived from omental adipose depots are refractory to the effects of TZDs. It has therefore been suggested that the insulin sensitizing effects of TZDs may result from their ability to promote adipogenesis in subcutaneous adipose depots (Adams et al., supra). Further, dominant negative mutations in the PPARγ gene have been identified in two non-obese subjects with severe insulin resistance, hypertension, and overt non-insulin dependent diabetes mellitus (NIDDM) (Barroso, I. et al. (1999) Nature 402:880-883).

NIDDM is the most common form of diabetes mellitus, a chronic metabolic disease that affects 143 million people worldwide. NIDDM is characterized by abnormal glucose and lipid metabolism that results from a combination of peripheral insulin resistance and defective insulin secretion. NIDDM has a complex, progressive etiology and a high degree of heritability. Numerous complications of diabetes including heart disease, stroke, renal failure, retinopathy, and peripheral neuropathy contribute to the high rate of morbidity and mortality.

At the molecular level, PPARγ functions as a ligand activated transcription factor. In the presence of ligand, PPARγ forms a heterodimer with the retinoid X receptor (RXR) which then activates transcription of target genes containing one or more copies of a PPARγ response element (PPRE). Many genes important in lipid storage and metabolism contain PPREs and have been identified as PPARγ targets, including PEPCK, aP2, LPL, ACS, and FAT-P (Auwerx, J. (1999) Diabetologia 42:1033-1049). Multiple ligands for PPARγ have been identified. These include a variety of fatty acid metabolites; synthetic drugs belonging to the TZD class, such as Pioglitazone and Rosiglitazone (BRL49653); and certain non-glitazone tyrosine analogs such as GI262570 and GW1929. The prostaglandin derivative 15-dPGJ2 is a potent endogenous ligand for PPARγ.

Expression of PPARγ is very high in adipose but barely detectable in skeletal muscle, the primary site for insulin stimulated glucose disposal in the body. PPARγ is also moderately expressed in large intestine, kidney, liver, vascular smooth muscle, hematopoietic cells, and macrophages. The high expression of PPARγ in adipose tissue suggests that the insulin sensitizing effects of TZDs may result from alterations in the expression of one or more PPARγ regulated genes in adipose tissue. Identification of PPARγ target genes will contribute to better drug design and the development of novel therapeutic strategies for diabetes, obesity, and other conditions.

Systematic attempts to identify PPARγ target genes have been made in several rodent models of obesity and diabetes (Suzuki, A. et al. (2000) Jpn. J. Pharmacol. 84:113-123; Way, J. M. et al. (2001) Endocrinology 142:1269-1277). However, a serious drawback of the rodent gene expression studies is that significant differences exist between human and rodent models of adipogenesis, diabetes, and obesity (Taylor, S. I. (1999) Cell 97:9-12; Gregoire, F. M. et al. (1998) Physiol. Reviews 78:783-809). Therefore, an unbiased approach to identifying TZD regulated genes in primary cultures of human tissues is necessary to fully elucidate the molecular basis for diseases associated with PPARγ activity.

Tangier Disease

Tangier disease (TD) is a genetic disorder characterized by the near absence of circulating high density lipoprotein (HDL) and the accumulation of cholesterol esters in many tissues, including tonsils, lymph nodes, liver, spleen, thymus, and intestine. Low levels of HDL represent a clear predictor of premature coronary artery disease and homozygous TD correlates with a four- to six-fold increase in cardiovascular disease compared to controls. HDL plays a cardio-protective role in reverse cholesterol transport, the flux of cholesterol from peripheral cells such as tissue macrophages through plasma lipoproteins to the liver. The HDL protein, apolipoprotein A-I, plays a major role in this process, interacting with the cell surface to remove excess cholesterol and phospholipids. This pathway is severely impaired in TD and the defect lies in a specific gene, the ABC1 transporter. This gene is a member of the family of ATP-binding cassette transporters, which utilize ATP hydrolysis to transport a variety of substrates across membranes.

Atherosclerosis

Atherosclerosis is a pathological condition characterized by a chronic local inflammatory response within the vessel wall of major arteries. Disease progression results in the formation of atherosclerotic lesions, unstable plaques which occasionally rupture, precipitating a catastrophic thrombotic occlusion of the vessel lumen. Atherosclerosis and the associated coronary artery disease and cerebral stroke represent the most common causes of death in industrialized nations. Although certain key risk factors have been identified, a full molecular characterization that elucidates the causes and identifies all potential therapeutic targets for this complex disease has not been achieved. Molecular characterization of atherosclerosis requires identification of the genes that contribute to lesion growth, stability, dissolution, rupture and induction of occlusive vessel thrombi.

Development of atherosclerosis is understood to be induced by the presence of circulating lipoprotein. Lipoproteins, such as the cholesterol-rich low-density lipoprotein (LDL), accumulate in the extracellular space of the vascular intima, and undergo modification. Oxidation of LDL (Ox-LDL) occurs most avidly in the sub-endothelial space where circulating antioxidant defenses are less effective. Mononuclear phagocytes enter the intima, differentiate into macrophages, and ingest modified lipids including Ox-LDL. During Ox-LDL uptake, macrophages produce cytokines (e.g. TNF-α and IL-1) and growth factors (e.g. M-CSF, VEGF, and PDGF-BB) that elicit further cellular events that modulate atherogenesis such as smooth muscle cell proliferation and production of extracellular matrix by vascular endothelium. Additionally, these macrophages may activate genes in endothelium and smooth muscle tissue involved in inflammation and tissue differentiation, including superoxide dismutatse (SOD), IL-8, and ICAM-1.

The vascular endothelium influences not only the three classically interacting components of hemostasis: the vessel, the blood platelets and the clotting and fibrinolytic systems of plasma, but also the natural sequelae: inflammation and tissue repair. Two principal modes of endothelial behavior may be differentiated, best defined as an anti- and a pro-thrombotic state. Under physiological conditions endothelium mediates vascular dilatation (formation of nitric oxide (NO), PGI₂, adenosine, hyperpolarising factor), prevents platelet adhesion and activation (production of adenosine, NO and PGI₂, removal of ADP), blocks thrombin formation (tissue factor pathway inhibitor, activation of protein C via thrombomodulin, activation of antithrombin III) and mitigates fibrin deposition (t- and scuplasminogen activator production). Adhesion and transmigration of inflammatory leukocytes are attenuated, e.g. by NO and IL-10, and oxygen radicals are efficiently scavenged (urate, NO, glutathione, SOD).

Activation of vascular endothelium is a central event in a wide range of physiological and disease processes such as vascular tone regulation, coagulation and thrombosis, atherosclerosis, inflammation and some infectious diseases. Blood vessel walls are composed of two tissue layers: an endothelial cell (EC) layer which comprises the lumenal surface of the vessel, and an underlying vascular smooth muscle cell (VSMC) layer. Through dynamic interactions with each other and with surrounding tissues, the vascular endothelium and smooth muscle tissues maintain vascular tone, control selective permeability of the vascular wall, direct vessel remodeling and angiogenesis, and modulate inflammatory and immune responses.

Non-atherosclerotic vascular endothelium not only mediates vascular dilatation but prevents platelet adhesion and activation, blocks thrombin formation, mitigates fibrin deposition, and attenuates adhesion and transmigration of inflammatory leukocytes. When the endothelium is physically disrupted, or perturbed by events such as postischemic reperfusion, acute and chronic inflammation, atherosclerosis, diabetes and chronic arterial hypertension, it acts in the opposite manner. The perturbed or proinflammatory state is characterized by vaso-constriction, platelet and leukocyte activation and adhesion (involving externalization, expression and upregulation of, for example, von Willebrand factor, platelet activating factor, P-selectin, ICAM-1, IL-8, MCP-1, and TNF-α), promotion of thrombin formation, coagulation and deposition of fibrin at the vascular wall (expression of tissue factor, PAI-1, and phosphatidyl serine) and, in platelet-leukocyte coaggregates, additional inflammatory interactions via attachment of platelet CD40-ligand to endothelial, monocyte and B-cell CD40. Thrombin formation and inflammatory stimulation set the stage for later tissue repair, but limiting procoagulatory, prothrombotic actions of a dysfunctional vascular endothelium may be the goal of clinical interventions (Becker, B. F. et al. (2000) Z. Kardiol. 89:160-167).

Treatment of confluent cultures of vascular smooth muscle cells (SMCs) with TNF-α suppresses the incorporation of [³H]proline into both collagenase-digestible proteins (CDP) and noncollagenous proteins (NCP). Such suppression by TNF-α is not observed in confluent bovine aortic endothelial cells and human fibroblastic IMR-90 cells. TNF-α decreases the relative proportion of collagen types IV and V suggesting that TNF-α modulates collagen synthesis by SMCs depending on their cell density and therefore may modify formation of atherosclerotic lesions (Hiraga, S. et al. (2000) Life Sci. 66:235-244).

Human coronary artery smooth muscle cells (CASMC) are primary cells isolated from the tunica media (an intermediate muscular layer) of a human coronary artery. Vascular smooth muscle cells are a model of increasing significance in vascular biology. It is now well known that besides their obvious role in the regulation of vascular tone and, consequently, oxygen supply to various tissues, their behavior under inflammatory conditions is an important factor in the development of atherosclerosis and restenosis.

Human aortic endothelial cells (HAECs) are primary cells derived from the endothelium of a human aorta. HAECs have been used as an experimental model for investigating in vitro the role of the endothelium in human vascular biology. Activation of the vascular endothelium is considered to be a central event in a wide range of both physiological and pathophysiological processes, such as vascular tone regulation, coagulation and thrombosis, atherosclerosis, and inflammation.

Thus, vascular tissue genes differentially expressed during treatment of vascular smooth muscle cells and vascular endothelial cell cultures with TNF-α may reasonably be expected to be markers of the atherosclerotic process.

Several investigators have examined changes in vascular cell gene expression associated with various inflammatory diseases or model systems. Examining human umbilical vein endothelial cells (HUVEC) activated by recombinant TNF-α or conditioned medium from activated human primary monocytes, Horrevoets et al. (1999; Blood 93:3418-3431) identified 106 differentially regulated genes. In a similar approach, deVries et al. (2000; J. Biol. Chem. 275:23939-23947) identified 40 differentially regulated genes in umbilical cord artery-derived smooth muscle cells activated by conditioned media from cultured macrophages after stimulation with oxidized LDL particles. In both studies, many of the identified genes were already known to be involved in inflammation. Comparing expression profiles from inflammatory diseased tissues, cultured macrophages, chondrocyte cell lines, primary chondrocytes, and synoviocytes, Heller et al. (1997; Proc. Natl. Acad. Sci. USA 94:2150-2155) identified candidate genes involved in inflammatory responses, including TNF, IL-1 IL-6, IL-8 G-CSF, RANTES, and V-CAM. From this candidate gene set, tissue inhibitor of metalloproteinase 1, ferritin light chain, and manganese superoxide dismutase were found to be differentially expressed in rheumatoid arthritis (RA) relative to inflammatory bowel disease (IBD). Further, IL-3, chemokine Groα, and metalloproteinase matrix metallo-elastase were expressed in both RA and IBD. Most recently, in an analysis of cultured aortic smooth muscle cells treated with TNF-α, Haley et al. (2000; Circulation 102:2185-2189) found a 20-fold increase in eotaxin, an eosinophil chemotactic factor. The overexpression of eotaxin and its receptor CCR3 in atherosclerotic lesions was confirmed by northern analysis.

Inflammation

The inflammatory response is a complex vascular reaction mediated by numerous cytokines, chemokines, growth factors, and other signaling molecules expressed by activated ECs, VSMCs and leukocytes. Inflammation protects the organism during trauma and infection, but can also lead to pathological conditions such as atherosclerosis. The pro-inflammatory cytokines, interleukin (IL)-1 and tumor necrosis factor (TNF), are secreted by a small number of activated macrophages or other cells and can set off a cascade of vascular changes, largely through their ability to alter gene expression patterns in ECs and VSMCs. These vascular changes include vasodilation and increased permeability of microvasculature, edema, and leukocyte extravasation and transmigration across the vessel wall. Ultimately, leukocytes, particularly neutrophils and monocytes/macrophages, accumulate in the extravascular space, where they remove injurious agents by phagocytosis and oxidative killing, a process accompanied by release of toxic factors, such as proteases and reactive oxygen species.

IL-1β and TNF-α induce pro-inflammatory, thrombotic, and anti-apoptotic changes in gene expression by signaling through receptors on the surface of ECs and VSMCs; these receptors activate transcription factors such as NF-κB as well as AP-1, IRF-1, and NF-GMa, leading to alterations in gene expression. Genes known to be differentially regulated in EC by IL-1β and TNF-α include E-selectin, VCAM-1, ICAM-1, PAF, Iκ-Bα, IAP-1, MCP-1, eotaxin, ENA-78, G-CSF, A20, ICE, and complement C3 component. A key event in inflammation, adhesion and transmigration of blood leukocytes across the vascular endothelium, for example, is mediated by increased expression of E-selectin, P-selectin, ICAM-1, and VCAM-1 on activated endothelium.

Tumor necrosis factor α is a pleiotropic cytokine that a mediates immune regulation and inflammatory responses. TNF-α-related cytokines generate partially overlapping cellular responses, including differentiation, proliferation, nuclear factor-κB (NF-κB) activation, and cell death, by triggering the aggregation of receptor monomers (Smith, C. A. et al. (1994) Cell 76:959-962). The cellular responses triggered by TNF-α are initiated through its interaction with distinct cell surface receptors (TNFRs). NF-κB is a transcription factor with a pivotal role in inducing genes involved in physiological processes as well as in the response to injury and infection. Activation of NF-κB involves the phosphorylation and subsequent degradation of an inhibitory protein, Iκ-B, and many of the proximal kinases and adaptor molecules involved in this process have been elucidated. Additionally, the NF-κB activation pathway from cell membrane to nucleus for IL-1 and TNF-α is now understood (Bowie, A. G. and L. A. O'Neill (2000) Biochem. Pharmacol. 59:13-23).

TNF-α is upregulated when the endothelium is physically disrupted or functionally perturbed by events such as postischemic reperfusion, acute and chronic inflammation, atherosclerosis, diabetes and chronic arterial hypertension. Inflammatory stimulation sets the stage for later tissue repair. Elevated TNF-α initially increases, and then inhibits, the activity of a number of key enzymes including protein-tyrosine kinase (PTKase) and protein-tyrosine phosphatase (Holden, R. J. et al. (1999) Med. Hypotheses 52:319-23).

Interferon-gamma (IFN-γ), also known as Type II interferon or immune interferon, is a cytokine produced primarily by T-lymphocytes and natural killer cells. Mature IFN-γ exists as noncovalently- linked homodimers. IFN-γ displays antiviral, antiproliferative, immunoregulatory, and proinflammatory activities and is important in host defense mechanisms. IFN-γ induces the production of cytokines; upregulates the expression of class I and II MHC antigens, Fc receptor, and leukocyte adhesion molecule; modulates macrophage effector functions; influences isotype switching; potentiates the secretion of immunoglobulins by B cells; augments TH1 cell expansion; and may be required for TH1 cell differentiation. IFN-γ exerts its biological activities by binding to specific cell surface receptors, which display high affinity binding sites. The IFN-γ receptor is present on almost all cell types except mature erythrocytes. Upon binding to its receptor, IFN-γ triggers the activation of JAK-1 and JAK-2 kinases resulting in the phosphorylation of STAT1. Both IFN-γ and TNF-α are considered proinflammatory cytokines. Cross-talk can exist between the signal transduction pathways of two cytokines; for example, signal transduction cascades initiated by two different cytokines lead to the activation of NF-κB.

Monocyte chemoattractant protein-1 (MCP-1) is known to play an important role in the pathogenesis of atherosclerosis by inducing monocyte migration. TNF-α treatment of human umbilical vein endothelial cells (HUVECs) increased the cellular secretions of MCP-1 119-fold compared with untreated cells. Troglitazone, an insulin-sensitizing drug, significantly inhibited this TNF-α-induced increase in MCP-1 secretions and decreased mRNA levels (Ohta, M. Y. et al. (2000) Diabetes Res. Clin. Pract. 48:171-176).

There is a need in the art for new compositions, including nucleic acids and proteins, for the diagnosis, prevention, and treatment of immune system, neurological, developmental, muscle, and cell proliferative disorders.

### SUMMARY OF THE INVENTION

Various embodiments of the invention provide a purified IRAP polypeptide, immune response associated proteins, referred to as ‘IRAP-17,’ and methods for using this protein and its encoding polynucleotides for the detection, diagnosis, and treatment of diseases and medical conditions. Embodiments also provide methods for utilizing the purified immune response associated protein and/or its encoding polynucleotides for facilitating the drug discovery process, including determination of efficacy, dosage, toxicity, and pharmacology. Related embodiments provide methods for utilizing the purified immune response associated protein and/or its encoding polynucleotides for investigating the pathogenesis of diseases and medical conditions.

An embodiment provides an isolated polypeptide selected from the group consisting of a) a polypeptide comprising an amino acid sequence set forth in SEQ ID NO: 1, b) a polypeptide comprising a naturally occurring amino acid sequence at least 90% identical or at least about 90% identical to an amino acid sequence set forth in SEQ ID NO: 1, c) a biologically active fragment of a polypeptide having an amino acid sequence set forth in SEQ ID NO: 1, and d) an immunogenic fragment of a polypeptide having an amino acid sequence set forth in SEQ ID NO: 1. Another embodiment provides an isolated polypeptide comprising an amino acid sequence set forth in SEQ ID NO: 1.

Still another embodiment provides an isolated polynucleotide encoding a polypeptide selected from the group consisting of a) a polypeptide comprising an amino acid sequence set forth in SEQ ID NO: 1, b) a polypeptide comprising a naturally occurring amino acid sequence at least 90% identical or at least about 90% identical to an amino acid sequence set forth in SEQ ID NO: 1, c) a biologically active fragment of a polypeptide having an amino acid sequence set forth in SEQ ID NO: 1, and d) an immunogenic fragment of a polypeptide having an amino acid sequence set forth in SEQ ID NO: 1. In another embodiment, the polynucleotide encodes a polypeptide set forth in SEQ ID NO: 1. In an alternative embodiment, the polynucleotide is set forth in SEQ ID NO: 2.

Still another embodiment provides a recombinant polynucleotide comprising a promoter sequence operably linked to a polynucleotide encoding a polypeptide selected from the group consisting of a) a polypeptide comprising an amino acid sequence set forth in SEQ ID NO: 1, b) a polypeptide comprising a naturally occurring amino acid sequence at least 90% identical or at least about 90% identical to an amino acid sequence set forth in SEQ ID NO: 1, c) a biologically active fragment of a polypeptide having an amino acid sequence set forth in SEQ ID NO: 1, and d) an immunogenic fragment of a polypeptide having an amino acid sequence set forth in SEQ ID NO: 1. Another embodiment provides a cell transformed with the recombinant polynucleotide. Yet another embodiment provides a transgenic organism comprising the recombinant polynucleotide.

Another embodiment provides a method for producing a polypeptide selected from the group consisting of a) a polypeptide comprising an amino acid sequence set forth in SEQ ID NO: 1, b) a polypeptide comprising a naturally occurring amino acid sequence at least 90% identical or at least about 90% identical to an amino acid sequence set forth in SEQ ID NO: 1, c) a biologically active fragment of a polypeptide having an amino acid sequence set forth in SEQ ID NO: 1, and d) an immunogenic fragment of a polypeptide having an amino acid sequence set forth in SEQ ID NO: 1. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Yet another embodiment provides an isolated antibody which specifically binds to a polypeptide selected from the group consisting of a) a polypeptide comprising an amino acid sequence set forth in SEQ ID NO: 1, b) a polypeptide comprising a naturally occurring amino acid sequence at least 90% identical or at least about 90% identical to an amino acid sequence set forth in SEQ ID NO: 1, c) a biologically active fragment of a polypeptide having an amino acid sequence set forth in SEQ ID NO: 1, and d) an immunogenic fragment of a polypeptide having an amino acid sequence set forth in SEQ ID NO: 1.

Still yet another embodiment provides an isolated polynucleotide selected from the group consisting of a) a polynucleotide comprising a polynucleotide sequence set forth in SEQ ID NO: 2, b) a polynucleotide comprising a naturally occurring polynucleotide sequence at least 90% identical or at least about 90% identical to a polynucleotide sequence set forth in SEQ ID NO: 2, c) a polynucleotide complementary to the polynucleotide of a), d) a polynucleotide complementary to the polynucleotide of b), and e) an RNA equivalent of a)-d). In other embodiments, the polynucleotide can comprise at least about 20, 30, 40, 60, 80, or 100 contiguous nucleotides.

Yet another embodiment provides a method for detecting a target polynucleotide in a sample, said target polynucleotide being selected from the group consisting of a) a polynucleotide comprising a polynucleotide sequence set forth in SEQ ID NO: 2, b) a polynucleotide comprising a naturally occurring polynucleotide sequence at least 90% identical or at least about 90% identical to a polynucleotide sequence set forth in SEQ ID NO: 2, c) a polynucleotide complementary to the polynucleotide of a), d) a polynucleotide complementary to the polynucleotide of b), and e) an RNA equivalent of a)-d). The method comprises a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and b) detecting the presence or absence of said hybridization complex. In a related embodiment, the method can include detecting the amount of the hybridization complex. In still other embodiments, the probe can comprise at least about 20, 30, 40, 60, 80, or 100 contiguous nucleotides.

Still yet another embodiment provides a method for detecting a target polynucleotide in a sample, said target polynucleotide being selected from the group consisting of a) a polynucleotide comprising a polynucleotide sequence set forth in SEQ ID NO: 2, b) a polynucleotide comprising a naturally occurring polynucleotide sequence at least 90% identical or at least about 90% identical to a polynucleotide sequence set forth in SEQ ID NO: 2, c) a polynucleotide complementary to the polynucleotide of a), d) a polynucleotide complementary to the polynucleotide of b), and e) an RNA equivalent of a)-d). The method comprises a) amplifying said target polynucleotide or fragment thereof using polymerase chain reaction amplification, and b) detecting the presence or absence of said amplified target polynucleotide or fragment thereof. In a related embodiment, the method can include detecting the amount of the amplified target polynucleotide or fragment thereof.

Another embodiment provides a composition comprising an effective amount of a polypeptide selected from the group consisting of a) a polypeptide comprising an amino acid sequence set forth in SEQ ID NO: 1, b) a polypeptide comprising a naturally occurring amino acid sequence at least 90% identical or at least about 90% identical to an amino acid sequence set forth in SEQ ID NO: 1, c) a biologically active fragment of a polypeptide having an amino acid sequence set forth in SEQ ID NO: 1, and d) an immunogenic fragment of a polypeptide having an amino acid sequence set forth in SEQ ID NO: 1, and a pharmaceutically acceptable excipient. In one embodiment, the composition can comprise an amino acid sequence set forth in SEQ ID NO: 1. Other embodiments provide a method of treating a disease or condition associated with decreased or abnormal expression of functional IRAP, comprising administering to a patient in need of such treatment the composition.

Yet another embodiment provides a method for screening a compound for effectiveness as an agonist of a polypeptide selected from the group consisting of a) a polypeptide comprising an amino acid sequence set forth in SEQ ID NO: 1, b) a polypeptide comprising a naturally occurring amino acid sequence at least 90% identical or at least about 90% identical to an amino acid sequence set forth in SEQ ID NO: 1, c) a biologically active fragment of a polypeptide having an amino acid sequence set forth in SEQ ID NO: 1, and d) an immunogenic fragment of a polypeptide having an amino acid sequence set forth in SEQ ID NO: 1. The method comprises a) contacting a sample comprising the polypeptide with a compound, and b) detecting agonist activity in the sample. Another embodiment provides a composition comprising an agonist compound identified by the method and a pharmaceutically acceptable excipient. Yet another embodiment provides a method of treating a disease or condition associated with decreased expression of functional IRAP, comprising administering to a patient in need of such treatment the composition.

Still yet another embodiment provides a method for screening a compound for effectiveness as an antagonist of a polypeptide selected from the group consisting of a) a polypeptide comprising an amino acid sequence set forth in SEQ ID NO: 1, b) a polypeptide comprising a naturally occurring amino acid sequence at least 90% identical or at least about 90% identical to an amino acid sequence set forth in SEQ ID NO: 1, c) a biologically active fragment of a polypeptide having an amino acid sequence set forth in SEQ ID NO: 1, and d) an immunogenic fragment of a polypeptide having an amino acid sequence set forth in SEQ ID NO: 1. The method comprises a) contacting a sample comprising the polypeptide with a compound, and b) detecting antagonist activity in the sample. Another embodiment provides a composition comprising an antagonist compound identified by the method and a pharmaceutically acceptable excipient. Yet another embodiment provides a method of treating a disease or condition associated with overexpression of functional IRAP, comprising administering to a patient in need of such treatment the composition.

Another embodiment provides a method of screening for a compound that specifically binds to a polypeptide selected from the group consisting of a) a polypeptide comprising an amino acid sequence set forth in SEQ ID NO: 1, b) a polypeptide comprising a naturally occurring amino acid sequence at least 90% identical or at least about 90% identical to an amino acid sequence set forth in SEQ ID NO: 1, c) a biologically active fragment of a polypeptide having an amino acid sequence set forth in SEQ ID NO: 1, and d) an immunogenic fragment of a polypeptide having an amino acid sequence set forth in SEQ ID NO: 1. The method comprises a) combining the polypeptide with at least one test compound under suitable conditions, and b) detecting binding of the polypeptide to the test compound, thereby identifying a compound that specifically binds to the polypeptide.

Yet another embodiment provides a method of screening for a compound that modulates the activity of a polypeptide selected from the group consisting of a) a polypeptide comprising an amino acid sequence set forth in SEQ ID NO: 1, b) a polypeptide comprising a naturally occurring amino acid sequence at least 90% identical or at least about 90% identical to an amino acid sequence set forth in SEQ ID NO: 1, c) a biologically active fragment of a polypeptide having an amino acid sequence set forth in SEQ ID NO: 1, and d) an immunogenic fragment of a polypeptide having an amino acid sequence set forth in SEQ ID NO: 1. The method comprises a) combining the polypeptide with at least one test compound under conditions permissive for the activity of the polypeptide, b) assessing the activity of the polypeptide in the presence of the test compound, and c) comparing the activity of the polypeptide in the presence of the test compound with the activity of the polypeptide in the absence of the test compound, wherein a change in the activity of the polypeptide in the presence of the test compound is indicative of a compound that modulates the activity of the polypeptide.

Still yet another embodiment provides a method for screening a compound for effectiveness in altering expression of a target polynucleotide, wherein said target polynucleotide comprises a polynucleotide sequence set forth in SEQ ID NO: 2, the method comprising a) contacting a sample comprising the target polynucleotide with a compound, b) detecting altered expression of the target polynucleotide, and c) comparing the expression of the target polynucleotide in the presence of varying amounts of the compound and in the absence of the compound.

Another embodiment provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide selected from the group consisting of i) a polynucleotide comprising a polynucleotide sequence set forth in SEQ ID NO: 2, ii) a polynucleotide comprising a naturally occurring polynucleotide sequence at least 90% identical or at least about 90% identical to a polynucleotide sequence set forth in SEQ ID NO: 2, iii) a polynucleotide having a sequence complementary to i), iv) a polynucleotide complementary to the polynucleotide of ii), and v) an RNA equivalent of i)-iv). Hybridization occurs under conditions whereby a specific hybridization complex is formed between said probe and a target polynucleotide in the biological sample, said target polynucleotide selected from the group consisting of i) a polynucleotide comprising a polynucleotide sequence set forth in SEQ ID NO: 2, ii) a polynucleotide comprising a naturally occurring polynucleotide sequence at least 90% identical or at least about 90% identical to a polynucleotide sequence set forth in SEQ ID NO: 2, iii) a polynucleotide complementary to the polynucleotide of i), iv) a polynucleotide complementary to the polynucleotide of ii), and v) an RNA equivalent of i)-iv). Alternatively, the target polynucleotide can comprise a fragment of a polynucleotide selected from the group consisting of i)-v) above; c) quantifying the amount of hybridization complex; and d) comparing the amount of hybridization complex in the treated biological sample with the amount of hybridization complex in an untreated biological sample, wherein a difference in the amount of hybridization complex in the treated biological sample is indicative of toxicity of the test compound.

### BRIEF DESCRIPTION OF THE TABLES

Table 1 summarizes the nomenclature for full length polynucleotide and polypeptide embodiments of the invention.

Table 2 shows the GenBank identification number and annotation of the nearest GenBank homolog, and the PROTEOME database identification numbers and annotations of PROTEOME database homologs, for polypeptide embodiments of the invention. The probability scores for the matches between each polypeptide and its homolog(s) are also shown.

Table 3 shows structural features of polypeptide embodiments, including predicted motifs and domains, along with the methods, algorithms, and searchable databases used for analysis of the polypeptides.

Table 4 lists the cDNA and/or genomic DNA fragments which were used to assemble polynucleotide embodiments, along with selected fragments of the polynucleotides.

Table 5 shows representative cDNA libraries for polynucleotide embodiments.

Table 6 provides an appendix which describes the tissues and vectors used for construction of the cDNA libraries shown in Table 5.

Table 7 shows the tools, programs, and algorithms used to analyze polynucleotides and polypeptides, along with applicable descriptions, references, and threshold parameters.

Table 8 shows single nucleotide polymorphisms found in polynucleotide sequences of the invention, along with allele frequencies in different human populations.

### DESCRIPTION OF THE INVENTION

Before the present proteins, nucleic acids, and methods are described, it is understood that embodiments of the invention are not limited to the particular machines, instruments, materials, and methods described, as these may vary. It is also to be understood that the terminology used herein is for the purpose of describing particular embodiments only, and is not intended to limit the scope of the invention.

As used herein and in the appended claims, the singular forms ‘a,’ ‘an,’ and ‘the’ include plural reference unless the context clearly dictates otherwise. Thus, for example, a reference to ‘a host cell’ includes a plurality of such host cells, and a reference to ‘an antibody’ is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with various embodiments of the invention. Nothing herein is to be construed as an admission that the invention is not entitled to antedate such disclosure by virtue of prior invention.

Definitions

‘IRAP’ refers to the amino acid sequences of substantially purified IRAP obtained from any species, particularly a mammalian species, including bovine, ovine, porcine, murine, equine, and human, and from any source, whether natural, synthetic, semi-synthetic, or recombinant.

The term ‘agonist’ refers to a molecule which intensifies or mimics the biological activity of IRAP. Agonists may include proteins, nucleic acids, carbohydrates, small molecules, or any other compound or composition which modulates the activity of IRAP either by directly interacting with IRAP or by acting on components of the biological pathway in which IRAP participates.

‘allelic variant’ is an alternative form of the gene encoding IRAP. Allelic variants may result from at least one mutation in the nucleic acid sequence and may result in altered mRNAs or in polypeptides whose structure or function may or may not be altered. A gene may have none, one, or many allelic variants of its naturally occurring form. Common mutational changes which give rise to allelic variants are generally ascribed to natural deletions, additions, or substitutions of nucleotides. Each of these types of changes may occur alone, or in combination with the others, one or more times in a given sequence.

‘Altered’ nucleic acid sequences encoding IRAP include those sequences with deletions, insertions, or substitutions of different nucleotides, resulting in a polypeptide the same as IRAP or a polypeptide with at least one functional characteristic of IRAP. Included within this definition are polymorphisms which may or may not be readily detectable using a particular oligonucleotide probe of the polynucleotide encoding IRAP, and improper or unexpected hybridization to allelic variants, with a locus other than the normal chromosomal locus for the polynucleotide encoding IRAP. The encoded protein may also be ‘altered,’ and may contain deletions, insertions, or substitutions of amino acid residues which produce a silent change and result in a functionally equivalent IRAP. Deliberate amino acid substitutions may be made on the basis of one or more similarities in polarity, charge, solubility, hydrophobicity, hydrophilicity, and/or the amphipathic nature of the residues, as long as the biological or immunological activity of IRAP is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

The terms ‘amino acid’ and ‘amino acid sequence’ can refer to an oligopeptide, a peptide, a polypeptide, or a protein sequence, or a fragment of any of these, and to naturally occurring or synthetic molecules. Where ‘amino acid sequence’ is recited to refer to a sequence of a naturally occurring protein molecule, ‘amino acid sequence’ and like terms are not meant to limit the amino acid sequence to the complete native amino acid sequence associated with the recited protein molecule.

‘Amplification’ relates to the production of additional copies of a nucleic acid. Amplification may be carried out using polymerase chain reaction (PCR) technologies or other nucleic acid amplification technologies well known in the art.

The term ‘antagonist’ refers to a molecule which inhibits or attenuates the biological activity of IRAP. Antagonists may include proteins such as antibodies, anticalins, nucleic acids, carbohydrates, small molecules, or any other compound or composition which modulates the activity of IRAP either by directly interacting with IRAP or by acting on components of the biological pathway in which IRAP participates.

The term ‘antibody’ refers to intact immunoglobulin molecules as well as to fragments thereof, such as Fab, F(ab′)₂, and Fv fragments, which are capable of binding an epitopic determinant. Antibodies that bind IRAP polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term ‘antigenic determinant’ refers to that region of a molecule (i.e., an epitope) that makes contact with a particular antibody. When a protein or a fragment of a protein is used to immunize a host animal, numerous regions of the protein may induce the production of antibodies which bind specifically to antigenic determinants (particular regions or three-dimensional structures on the protein). An antigenic determinant may compete with the intact antigen (i.e., the immunogen used to elicit the immune response) for binding to an antibody.

The term ‘aptamer’ refers to a nucleic acid or oligonucleotide molecule that binds to a specific molecular target. Aptamers are derived from an in vitro evolutionary process (e.g., SELEX (Systematic Evolution of Ligands by EXponential Enrichment), described in U.S. Pat. No. 5,270,163), which selects for target-specific aptamer sequences from large combinatorial libraries. Aptamer compositions may be double-stranded or single-stranded, and may include deoxyribonucleotides, ribonucleotides, nucleotide derivatives, or other nucleotide-like molecules. The nucleotide components of an aptamer may have modified sugar groups (e.g., the 2′-OH group of a ribonucleotide may be replaced by 2′-F or 2′-NH₂), which may improve a desired property, e.g., resistance to nucleases or longer lifetime in blood. Aptamers may be conjugated to other molecules, e.g., a high molecular weight carrier to slow clearance of the aptamer from the circulatory system. Aptamers may be specifically cross-linked to their cognate ligands, e.g., by photo-activation of a cross-linker (Brody, E. N. and L. Gold (2000) J. Biotechnol. 74:5-13).

The term ‘intramer’ refers to an aptamer which is expressed in vivo. For example, a vaccinia virus-based RNA expression system has been used to express specific RNA aptamers at high levels in the cytoplasm of leukocytes (Blind, M. et al. (1999) Proc. Natl. Acad. Sci. USA 96:3606-3610).

The term ‘spiegelmer’ refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides.

The term ‘antisense’ refers to any composition capable of base-pairing with the ‘sense’ (coding) strand of a polynucleotide having a specific nucleic acid sequence. Antisense compositions may include DNA; RNA; peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may be produced by any method including chemical synthesis or transcription. Once introduced into a cell, the complementary antisense molecule base-pairs with a naturally occurring nucleic acid sequence produced by the cell to form duplexes which block either transcription or translation. The designation ‘negative’ or ‘minus’ can refer to the antisense strand, and the designation ‘positive’ or ‘plus’ can refer to the sense strand of a reference DNA molecule.

The term ‘biologically active’ refers to a protein having structural, regulatory, or biochemical functions of a naturally occurring molecule. Likewise, ‘immunologically active’ or ‘immunogenic’ refers to the capability of the natural, recombinant, or synthetic IRAP, or of any oligopeptide thereof, to induce a specific immune response in appropriate animals or cells and to bind with specific antibodies.

‘Complementary’ describes the relationship between two single-stranded nucleic acid sequences that anneal by base-pairing. For example, 5′-AGT-3′pairs with its complement,

3′-TCA-5′.

A ‘composition comprising a given polynucleotide’ and a ‘composition comprising a given polypeptide’ can refer to any composition containing the given polynucleotide or polypeptide. The composition may comprise a dry formulation or an aqueous solution. Compositions comprising polynucleotides encoding IRAP or fragments of IRAP may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g., sodium dodecyl sulfate; SDS), and other components (e.g., Denhardt's solution, dry milk, salmon sperm DNA, etc.).

‘Consensus sequence’ refers to a nucleic acid sequence which has been subjected to repeated DNA sequence analysis to resolve uncalled bases, extended using the XL-PCR kit (Applied Biosystems, Foster City Calif.) in the 5′ and/or the 3′ direction, and resequenced, or which has been assembled from one or more overlapping cDNA, EST, or genomic DNA fragments using a computer program for fragment assembly, such as the GELVIEW fragment assembly system (Accelrys, Burlington MA) or Phrap (University of Washington, Seattle Wash.). Some sequences have been both extended and assembled to produce the consensus sequence.

‘Conservative amino acid substitutions’ are those substitutions that are predicted to least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A ‘deletion’ refers to a change in the amino acid or nucleotide sequence that results in the absence of one or more amino acid residues or nucleotides.

The term ‘derivative’ refers to a chemically modified polynucleotide or polypeptide. Chemical modifications of a polynucleotide can include, for example, replacement of hydrogen by an alkyl, acyl, hydroxyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A ‘detectable label’ refers to a reporter molecule or enzyme that is capable of generating a measurable signal and is covalently or noncovalently joined to a polynucleotide or polypeptide.

‘Differential expression’ refers to increased or upregulated; or decreased, downregulated, or absent gene or protein expression, determined by comparing at least two different samples. Such comparisons may be carried out between, for example, a treated and an untreated sample, or a diseased and a normal sample.

‘Exon shuffling’ refers to the recombination of different coding regions (exons). Since an exon may represent a structural or functional domain of the encoded protein, new proteins may be assembled through the novel reassortment of stable substructures, thus allowing acceleration of the evolution of new protein functions.

A ‘fragment’ is a unique portion of IRAP or a polynucleotide encoding IRAP which can be identical in sequence to, but shorter in length than, the parent sequence. A fragment may comprise up to the entire length of the defined sequence, minus one nucleotide/amino acid residue. For example, a fragment may comprise from about 5 to about 1000 contiguous nucleotides or amino acid residues. A fragment used as a probe, primer, antigen, therapeutic molecule, or for other purposes, may be at least 5, 10, 15, 16, 20, 25, 30, 40, 50, 60, 75, 100, 150, 250 or at least 500 contiguous nucleotides or amino acid residues in length. Fragments may be preferentially selected from certain regions of a molecule. For example, a polypeptide fragment may comprise a certain length of contiguous amino acids selected from the first 250 or 500 amino acids (or first 25% or 50%) of a polypeptide as shown in a certain defined sequence. Clearly these lengths are exemplary, and any length that is supported by the specification, including the Sequence Listing, tables, and figures, may be encompassed by the present embodiments.

A fragment of SEQ ID NO: 2 can comprise a region of unique polynucleotide sequence that specifically identifies SEQ ID NO: 2, for example, as distinct from any other sequence in the genome from which the fragment was obtained. A fragment of SEQ ID NO: 2 can be employed in one or more embodiments of methods of the invention, for example, in hybridization and amplification technologies and in analogous methods that distinguish SEQ ID NO: 2 from related polynucleotides. The precise length of a fragment of SEQ ID NO: 2 and the region of SEQ ID NO: 2 to which the fragment corresponds are routinely determinable by one of ordinary skill in the art based on the intended purpose for the fragment.

A fragment of SEQ ID NO: 1 is encoded by a fragment of SEQ ID NO: 2. A fragment of SEQ ID NO: 1 can comprise a region of unique amino acid sequence that specifically identifies SEQ ID NO: 1. For example, a fragment of SEQ ID NO: 1 can be used as an immunogenic peptide for the development of antibodies that specifically recognize SEQ ID NO: 1. The precise length of a fragment of SEQ ID NO: 1 and the region of SEQ ID NO: 1 to which the fragment corresponds can be determined based on the intended purpose for the fragment using one or more analytical methods described herein or otherwise known in the art.

A ‘full length’ polynucleotide is one containing at least a translation initiation codon (e.g., methionine) followed by an open reading frame and a translation termination codon. A ‘full length’ polynucleotide sequence encodes a ‘full length’ polypeptide sequence.

‘Homology’ refers to sequence similarity or, alternatively, sequence identity, between two or more polynucleotide sequences or two or more polypeptide sequences.

The terms ‘percent identity’ and ‘% identity,’ as applied to polynucleotide sequences, refer to the percentage of identical nucleotide matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using one or more computer algorithms or programs known in the art or described herein. For example, percent identity can be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and P. M. Sharp (1989; CABIOS 5:151-153) and in Higgins, D. G. et al. (1992; CABIOS 8:189-191). For pairwise alignments of polynucleotide sequences, the default parameters are set as follows: Ktuple=2, gap penalty=5, window=4, and ‘diagonals saved’=4. The ‘weighted’ residue weight table is selected as the default.

Alternatively, a suite of commonly used and freely available sequence comparison algorithms which can be used is provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at ncbi.nlm.nih.gov/BLAST/. The BLAST software suite includes various sequence analysis programs including ‘blastn,’ that is used to align a known polynucleotide sequence with other polynucleotide sequences from a variety of databases. Also available is a tool called ‘BLAST 2 Sequences’ that is used for direct pairwise comparison of two nucleotide sequences. ‘BLAST 2 Sequences’ can be accessed and used interactively at ncbi.nlm.nih.gov/gorf/bl2.html. The ‘BLAST 2 Sequences’ tool can be used for both blastn and blastp (discussed below). BLAST programs are commonly used with gap and other parameters set to default settings. For example, to compare two nucleotide sequences, one may use blastn with the ‘BLAST 2 Sequences’ tool Version 2.0.12 (Apr. 21, 2000) set at default parameters. Such default parameters may be, for example:
Matrix: BLOSUM62
Reward for match: 1
Penalty for mismatch: -2
Open Gap: 5 and Extension Gap: 2 penalties
Gap x drop-off: 50
Expect: 10
Word Size: 11
Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 con nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases ‘percent identity’ and ‘% identity,’ as applied to polypeptide sequences, refer to the percentage of identical residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. The phrases ‘percent similarity’ and ‘% similarity,’ as applied to polypeptide sequences, refer to the percentage of residue matches, including identical residue matches and conservative substitutions, between at least two polypeptide sequences aligned using a standardized algorithm. In contrast, conservative substitutions are not included in the calculation of percent identity between polypeptide sequences.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty=3, window=5, and ‘diagonals saved’=5. The PAM250 matrix is selected as the default residue weight table.

Alternatively the NCBI BLAST software suite may be used. For example, for a pairwise comparison of two polypeptide sequences, one may use the ‘BLAST 2 Sequences’ tool Version 2.0.12 (Apr.-21-2000) with blastp set at default parameters. Such default parameters may be, for example:
Matrix: BLOSUM62
Open Gap. 11 and Extension Gap: 1 penalties
Gap x drop-off 50
Expect: 10
Word Size: 3
Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or least 150 contiguous residues. Such lengths are exemplary only, and it is understood that any fragment length supported by the sequences shown herein, in the tables, figures or Sequence Listing, may be used to describe a length over which percentage identity may be measured.

‘Human artificial chromosomes’ (HACs) are linear microchromosomes which may contain DNA sequences of about 6 kb to 10 Mb in size and which contain all of the elements required for chromosome replication, segregation and maintenance.

The term ‘humanized antibody’ refers to an antibody molecule in which the amino acid sequence in the non-antigen binding regions has been altered so that the antibody more closely resembles a human antibody, and still retains its original binding ability.

‘Hybridization’ refers to the process by which a polynucleotide strand anneals with a complementary strand through base pairing under defined hybridization conditions. Specific hybridization is an indication that two nucleic acid sequences share a high degree of complementarity. Specific hybridization complexes form under permissive annealing conditions and remain hybridized after the ‘washing’ step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68□C in the presence of about 6×SSC, about 1% (w/v) SDS, and about 100 μg/ml sheared, denatured salmon sperm DNA.

Generally, stringency of hybridization is expressed, in part, with reference to the temperature under which the wash step is carried out. Such wash temperatures are typically selected to be about 51° C. to 201° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook, J. and D. W. Russell (2001; Molecular Cloning: A Laboratory Manual, 3rd ed., vol. 1-3, Cold Spring Harbor Press, Cold Spring Harbor N.Y., ch. 9).

High stringency conditions for hybridization between polynucleotides of the present invention include wash conditions of 68° C. in the presence of about 0.2×SSC and about 0.1 % SDS, for 1 hour. Alternatively, temperatures of about 65° C., 60° C., 55° C., or 42° C. may be used. SSC concentration may be varied from about 0.1 to 2×SSC, with SDS being present at about 0.1%. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

The term ‘hybridization complex’ refers to a complex formed between two nucleic acids by virtue of the formation of hydrogen bonds between complementary bases. A hybridization complex may be formed in solution (e.g., C₀t or R₀t analysis) or formed between one nucleic acid present in solution and another nucleic acid immobilized on a solid support (e.g., paper, membranes, filters, chips, pins or glass slides, or any other appropriate substrate to which cells or their nucleic acids have been fixed).

The words ‘insertion’ and ‘addition’ refer to changes in an amino acid or polynucleotide sequence resulting in the addition of one or more amino acid residues or nucleotides, respectively.

‘Immune response’ can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

An ‘immunogenic fragment’ is a polypeptide or oligopeptide fragment of IRAP which is capable of eliciting an immune response when introduced into a living organism, for example, a mammal. The term ‘immunogenic fragment’ also includes any polypeptide or oligopeptide fragment of IRAP which is useful in any of the antibody production methods disclosed herein or known in the art.

The term ‘microarray’ refers to an arrangement of a plurality of polynucleotides, polypeptides, antibodies, or other chemical compounds on a substrate.

The terms ‘element’ and ‘array element’ refer to a polynucleotide, polypeptide, antibody, or other chemical compound having a unique and defined position on a microarray.

The term ‘modulate’ refers to a change in the activity of IRAP. For example, modulation may cause an increase or a decrease in protein activity, binding characteristics, or any other biological, functional, or immunological properties of IRAP.

The phrases ‘nucleic acid’ and ‘nucleic acid sequence’ refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material.

‘Operably linked’ refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

‘Peptide nucleic acid’ (PNA) refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

‘Post-translational modification’ of an IRAP may involve lipidation, glycosylation, phosphorylation, acetylation, racemization, proteolytic cleavage, and other modifications known in the art. These processes may occur synthetically or biochemically. Biochemical modifications will vary by cell type depending on the enzymatic milieu of IRAP.

‘Probe’ refers to nucleic acids encoding IRAP, their complements, or fragments thereof, which are used to detect identical, allelic or related nucleic acids. Probes are isolated oligonucleotides or polynucleotides attached to a detectable label or reporter molecule. Typical labels include radioactive isotopes, ligands, chemiluminescent agents, and enzymes. ‘Primers’ are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 25, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the tables, figures, and Sequence Listing, may be used.

Methods for preparing and using probes and primers are described in, for example, Sambrook, J. and D. W. Russell (2001; Molecular Cloning: A Laboratory Manual, 3rd ed., vol. 1-3, Cold Spring Harbor Press, Cold Spring Harbor N.Y.), Ausubel, F. M. et al. (1999; Short Protocols in Molecular Biology, 4^{th} ed., John Wiley & Sons, New York N.Y.), and Innis, M. et al. (1990; PCR Protocols, A Guide to Methods and Applications, Academic Press, San Diego Calif.). PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/MIT Center for Genome Research, Cambridge Mass.) allows the user to input a ‘mispriming library,’ in which sequences to avoid as primer binding sites are user-specified. Primer3 is useful, in particular, for the selection of oligonucleotides for microarrays. (The source code for the latter two primer selection programs may also be obtained from their respective sources and modified to meet the users specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

A ‘recombinant nucleic acid’ is a nucleic acid that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook and Russell (supra). The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the mammal.

A ‘regulatory element’ refers to a nucleic acid sequence usually derived from untranslated regions of a gene and includes enhancers, promoters, introns, and 5′ and 3′ untranslated regions (UTRs). Regulatory elements interact with host or viral proteins which control transcription, translation, or RNA stability.

‘Reporter molecules’ are chemical or biochemical moieties used for labeling a nucleic acid, amino acid, or antibody. Reporter molecules include radionuclides; enzymes; fluorescent, chemiluminescent, or chromogenic agents; substrates; cofactors; inhibitors; magnetic particles; and other moieties known in the art.

An ‘RNA equivalent,’ in reference to a DNA molecule, is composed of the same linear sequence of nucleotides as the reference DNA molecule with the exception that all occurrences of the nitrogenous base thymine are replaced with uracil, and the sugar backbone is composed of ribose instead of deoxyribose.

The term ‘sample’ is used in its broadest sense. A sample suspected of containing IRAP, nucleic acids encoding IRAP, or fragments thereof may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms ‘specific binding’ and ‘specifically binding’ refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope ‘A,’ the presence of a polypeptide comprising the epitope A, or the presence of free unlabeled A, in a reaction containing free labeled A and the antibody will reduce the amount of labeled A that binds to the antibody.

The term ‘substantially purified’ refers to nucleic acid or amino acid sequences that are removed from their natural environment and are isolated or separated, and are at least about 60% free, preferably at least about 75% free, and most preferably at least about 90% free from other components with which they are naturally associated.

A ‘substitution’ refers to the replacement of one or more amino acid residues or nucleotides by different amino acid residues or nucleotides, respectively.

‘Substrate’ refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

A ‘transcript image’ or ‘expression profile’ refers to the collective pattern of gene expression by a particular cell type or tissue under given conditions at a given time.

‘Transformation’ describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term ‘transformed cells’ includes stably transformed cells in which the inserted DNA is capable of replication either as an autonomously replicating plasmid or as part of the host chromosome, as well as transiently transformed cells which express the inserted DNA or RNA for limited periods of time.

A ‘transgenic organism,’ as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. In another embodiment, the nucleic acid can be introduced by infection with a recombinant viral vector, such as a lentiviral vector (Lois, C. et al. (2002) Science 295:868-872). The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, plants and animals. The isolated DNA of the present invention can be introduced into the host by methods known in the art, for example infection, transfection, transformation or transconjugation. Techniques for transferring the DNA of the present invention into such organisms are widely known and provided in references such as Sambrook and Russell (supra).

A ‘variant’ of a particular nucleic acid sequence is defined as a nucleic acid sequence having at least 40% sequence identity to the particular nucleic acid sequence over a certain length of one of the nucleic acid sequences using blastn with the ‘BLAST 2 Sequences’ tool Version 2.0.9 (May-07-1999) set at default parameters. Such a pair of nucleic acids may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length. A variant may be described as, for example, an ‘allelic’ (as defined above), ‘splice,’ ‘species,’ or ‘polymorphic’ variant. A splice variant may have significant identity to a reference molecule, but will generally have a greater or lesser number of polynucleotides due to alternate splicing during mRNA processing. The corresponding polypeptide may possess additional functional domains or lack domains that are present in the reference molecule. Species variants are polynucleotides that vary from one species to another. The resulting polypeptides will generally have significant amino acid identity relative to each other. A polymorphic variant is a variation in the polynucleotide sequence of a particular gene between individuals of a given species. Polymorphic variants also may encompass ‘single nucleotide polymorphisms’ (SNPs) in which the polynucleotide sequence varies by one nucleotide base. The presence of SNPs may be indicative of, for example, a certain population, a disease state, or a propensity for a disease state.

A ‘variant’ of a particular polypeptide sequence is defined as a polypeptide sequence having at least 40% sequence identity or sequence similarity to the particular polypeptide sequence over a certain length of one of the polypeptide sequences using blastp with the ‘BLAST 2 Sequences’ tool Version 2.0.9 (May-07-1999) set at default parameters. Such a pair of polypeptides may show, for example, at least 50%, at least 60%, at least 70%, at least 80%, at least 85%, at least 90%, at least 91%, least 92%, at least 93%, at least 94%, at least 95%, at least 96%, at least 97%, at least 98%, or at least 99% or greater sequence identity or sequence similarity over a certain defined length of one of the polypeptides.

The Invention

Various embodiments of the invention include new human immune response associated proteins (IRAP), the polynucleotides encoding IRAP, and the use of these compositions for the diagnosis, treatment, or prevention of immune system, neurological, developmental, muscle, and cell proliferative disorders.

Table 1 summarizes the nomenclature for the full length polynucleotide and polypeptide embodiments of the invention. Each polynucleotide and its corresponding polypeptide are correlated to a single Incyte project identification number (Incyte Project ID). Each polypeptide sequence is denoted by both a polypeptide sequence identification number (Polypeptide SEQ ID NO:) and an Incyte polypeptide sequence number (Incyte Polypeptide ID) as shown. Each polynucleotide sequence is denoted by both a polynucleotide sequence identification number (Polynucleotide SEQ ID NO:) and an Incyte polynucleotide consensus sequence number (Incyte Polynucleotide ID) as shown. Column 6 shows the Incyte ID numbers of physical, full length clones corresponding to the polypeptide and polynucleotide sequences of the invention. The full length clones encode polypeptides which have at least 95% sequence identity to the polypeptide sequences shown in column 3.

Table 2 shows sequences with homology to polypeptide embodiments of the invention as identified by BLAST analysis against the GenBank protein (genpept) database and the PROTEOME database. Columns 1 and 2 show the polypeptide sequence identification number (Polypeptide SEQ ID NO:) and the corresponding Incyte polypeptide sequence number (Incyte Polypeptide ID) for polypeptides of the invention. Column 3 shows the GenBank identification number (GenBank ID NO:) of the nearest GenBank homolog and the PROTEOME database identification numbers (PROTEOME ID NO:) of the nearest PROTEOME database homologs. Column 4 shows the probability scores for the matches between each polypeptide and its homolog(s). Column 5 shows the annotation of the GenBank and PROTEOME database homolog(s) along with relevant citations where applicable, all of which are expressly incorporated by reference herein.

Table 3 shows various structural features of the polypeptides of the invention. Columns 1 and 2 show the polypeptide sequence identification number (SEQ ID NO:) and the corresponding Incyte polypeptide sequence number (Incyte Polypeptide ID) for each polypeptide of the invention. Column 3 shows the number of amino acid residues in each polypeptide. Column 4 shows potential phosphorylation sites and potential glycosylation sites, as determined by the MOTIFS program of the GCG sequence analysis software package (Accelrys, Burlington Mass.), as well as amino acid residues comprising signature sequences, domains, and motifs. Column 5 shows analytical methods for protein structure/function analysis and in some cases, searchable databases to which the analytical methods were applied.

Together, Tables 2 and 3 summarize the properties of polypeptides of the invention, and these properties establish that the claimed polypeptides are immune response associated proteins.

As an example, SEQ ID NO:1 is 94% identical, from residue M1 to residue T177, to human interleukin 6 (GenBank ID g186352) as determined by the Basic Local Alignment Search Tool (BLAST). (See Table 2.) The BLAST probability score is 3.1e-80, which indicates the probability of obtaining the observed polypeptide sequence alignment by chance. SEQ ID NO: 1 also has homology to cytokines that promote T and B cell proliferation and differentiation, and are implicated in multiple sclerosis and the persistence of multiple myeloma and chronic lymphocytic leukemia, as determined by BLAST analysis using the PROTEOME database. SEQ ID NO: 1 also contains an Interleukin-6 homologue or Interleukin-6/G-CSF/MGF family domain as determined by searching for statistically significant matches in the hidden Markov model (HMM)-based PFAM and SMART database of conserved protein families/domains. (See Table 3.) Data from BLIMPS and PROFILESCAN analyses, and BLAST analyses against the PRODOM and DOMO databases, provide further corroborative evidence that SEQ ID NO: 1 is an interleukin 6 family member or homologue.

The algorithms and parameters for the analysis of SEQ ID NO: 1 are described in Table 7.

As shown in Table 4, the full length polynucleotide embodiments were assembled using cDNA sequences or coding (exon) sequences derived from genomic DNA, or any combination of these two types of sequences. Column 1 lists the polynucleotide sequence identification number (Polynucleotide SEQ ID NO:), the corresponding Incyte polynucleotide consensus sequence number (Incyte ID) for each polynucleotide of the invention, and the length of each polynucleotide sequence in basepairs. Column 2 shows the nucleotide start (5′) and stop (3′) positions of the cDNA and/or genomic sequences used to assemble the full length polynucleotide embodiments, and of fragments of the polynucleotides which are useful, for example, in hybridization or amplification technologies that identify SEQ ID NO: 2 or that distinguish between SEQ ID NO: 2 and related polynucleotides.

The polynucleotide fragments described in Column 2 of Table 4 may refer specifically, for example, to Incyte cDNAs derived from tissue-specific cDNA libraries or from pooled cDNA libraries. Alternatively, the polynucleotide fragments described in column 2 may refer to GenBank cDNAs or ESTs which contributed to the assembly of the full length polynucleotides. In addition, the polynucleotide fragments described in column 2 may identify sequences derived from the ENSEMBL (The Sanger Centre, Cambridge, UK) database (i.e., those sequences including the designation ‘ENST’). Alternatively, the polynucleotide fragments described in column 2 may be derived from the NCBI RefSeq Nucleotide Sequence Records Database (i.e., those sequences including the designation ‘NM’ or ‘NT’) or the NCBI RefSeq Protein Sequence Records (i.e., those sequences including the designation ‘NP’). Alternatively, the polynucleotide fragments described in column 2 may refer to assemblages of both cDNA and Genscan-predicted exons brought together by an ‘exon stitching’ algorithm. For example, a polynucleotide sequence identified as FL₁₃XXXXXX_N_{1—}N_{2—}YYYYY_N_{3—}N₄ represents a ‘stitched’ sequence in which XXXXXX is the identification number of the cluster of sequences to which the algorithm was applied, and YYYYY is the number of the prediction generated by the algorithm, and N_{1,2,3. . .} , if present, represent specific exons that may have been manually edited during analysis (See Example V). Alternatively, the polynucleotide fragments in column 2 may refer to assemblages of exons brought together by an ‘exon-stretching’ algorithm. For example, a polynucleotide sequence identified as FLXXXXXX_gAAAAA_gBBBBB_{—}1_N is a ‘stretched’ sequence, with XXXXXX being the Incyte project identification number, gAAAAA being the GenBank identification number of the human genomic sequence to which the ‘exon-stretching’ algorithm was applied, gBBBBB being the GenBank identification number or NCBI RefSeq identification number of the nearest GenBank protein homolog, and N referring to specific exons (See Example V). In instances where a RefSeq sequence was used as a protein homolog for the ‘exon-stretching’ algorithm, a RefSeq identifier (denoted by ‘NM,’ ‘NP,’ or ‘NT’) may be used in place of the GenBank identifier (i.e., gBBBBB).

Alternatively, a prefix identifies component sequences that were hand-edited, predicted from genomic DNA sequences, or derived from a combination of sequence analysis methods. The following Table lists examples of component sequence prefixes and corresponding sequence analysis methods associated with the prefixes (see Example IV and Example V).

| Prefix | Type of analysis and/or examples of programs |
|---|---|
| GNN, GFG, | Exon prediction from genomic sequences using, for |
| ENST | example, GENSCAN (Stanford University, CA, USA) or |
| | FGENES (Computer Genomics Group, The Sanger Centre, |
| | Cambridge, UK). |
| GBI | Hand-edited analysis of genomic sequences. |
| FL | Stitched or stretched genomic sequences (see Example V). |
| INCY | Full length transcript and exon prediction from mapping |
| | of EST sequences to the genome. Genomic location and |
| | EST composition data are combined to predict the exons |
| | and resulting transcript. |

In some cases, Incyte cDNA coverage redundant with the sequence coverage shown in Table 4 was obtained to confirm the final consensus polynucleotide sequence, but the relevant Incyte cDNA identification numbers are not shown.

Table 5 shows the representative cDNA libraries for those full length polynucleotides which were assembled using Incyte cDNA sequences. The representative cDNA library is the Incyte cDNA library which is most frequently represented by the Incyte cDNA sequences which were used to assemble and confirm the above polynucleotides. The tissues and vectors which were used to construct the cDNA libraries shown in Table 5 are described in Table 6.

Table 8 shows single nucleotide polymorphisms (SNPs) found in polynucleotide sequences of the invention, along with allele frequencies in different human populations. Columns 1 and 2 show the polynucleotide sequence identification number (SEQ ID NO:) and the corresponding Incyte project identification number (PID) for polynucleotides of the invention. Column 3 shows the Incyte identification number for the EST in which the SNP was detected (EST ID), and column 4 shows the identification number for the SNP (SNP ID). Column 5 shows the position within the EST sequence at which the SNP is located (EST SNP), and column 6 shows the position of the SNP within the full-length polynucleotide sequence (CB1 SNP). Column 7 shows the allele found in the EST sequence. Columns 8 and 9 show the two alleles found at the SNP site. Column 10 shows the amino acid encoded by the codon including the SNP site, based upon the allele found in the EST. Columns 11-14 show the frequency of allele 1 in four different human populations. An entry of n/d (not detected) indicates that the frequency of allele 1 in the population was too low to be detected, while n/a (not available) indicates that the allele frequency was not determined for the population.

The invention also encompasses IRAP variants. Various embodiments of IRAP variants can have at least about 80%, at least about 90%, or at least about 95% amino acid sequence identity to the IRAP amino acid sequence, and can contain at least one functional or structural characteristic of IRAP.

Various embodiments also encompass polynucleotides which encode IRAP. In a particular embodiment, the invention encompasses a polynucleotide sequence comprising a sequence selected from the group consisting of SEQ ID NO: 2, which encodes IRAP. The polynucleotide sequences of SEQ ID NO: 2, as presented in the Sequence Listing, embrace the equivalent RNA sequences, wherein occurrences of the nitrogenous base thymine are replaced with uracil, and the sugar backbone is composed of ribose instead of deoxyribose.

The invention also encompasses variants of a polynucleotide encoding IRAP. In particular, such a variant polynucleotide will have at least about 70%, or alternatively at least about 85%, or even at least about 95% polynucleotide sequence identity to a polynucleotide encoding IRAP. A particular aspect of the invention encompasses a variant of a polynucleotide comprising a sequence selected from the group consisting of SEQ ID NO: 2 which has at least about 70%, or alternatively at least about 85%, or even at least about 95% polynucleotide sequence identity to a nucleic acid sequence selected from the group consisting of SEQ ID NO: 2. Any one of the polynucleotide variants described above can encode a polypeptide which contains at least one functional or structural characteristic of IRAP.

In addition, or in the alternative, a polynucleotide variant of the invention is a splice variant of a polynucleotide encoding IRAP. A splice variant may have portions which have significant sequence identity to a polynucleotide encoding IRAP, but will generally have a greater or lesser number of nucleotides due to additions or deletions of blocks of sequence arising from alternate splicing during mRNA processing. A splice variant may have less than about 70%, or alternatively less than about 60%, or alternatively less than about 50% polynucleotide sequence identity to a polynucleotide encoding IRAP over its entire length; however, portions of the splice variant will have at least about 70%, or alternatively at least about 85%, or alternatively at least about 95%, or alternatively 100% polynucleotide sequence identity to portions of the polynucleotide encoding IRAP.

It will be appreciated by those skilled in the art that as a result of the degeneracy of the genetic code, a multitude of polynucleotide sequences encoding IRAP, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring IRAP, and all such variations are to be considered as being specifically disclosed.

Although polynucleotides which encode IRAP and its variants are generally capable of hybridizing to polynucleotides encoding naturally occurring IRAP under appropriately selected conditions of stringency, it may be advantageous to produce polynucleotides encoding IRAP or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding IRAP and its derivatives without altering the encoded amino acid sequences include the production of RNA transcripts having more desirable properties, such as a greater half-life, than transcripts produced from the naturally occurring sequence.

The invention also encompasses production of polynucleotides which encode IRAP and IRAP derivatives, or fragments thereof, entirely by synthetic chemistry. After production, the synthetic polynucleotide may be inserted into any of the many available expression vectors and cell systems using reagents well known in the art. Moreover, synthetic chemistry may be used to introduce mutations into a polynucleotide encoding IRAP or any fragment thereof.

Embodiments of the invention can also include polynucleotides that are capable of hybridizing to the claimed polynucleotides, and, in particular, to those having the sequences shown in SEQ ID NO: 2 and fragments thereof, under various conditions of stringency (Wahl, G. M. and S. L. Berger (1987) Methods Enz mol. 152:399-407; Kimmel, A. R. (1987) Methods Enzymol. 152:507-511). Hybridization conditions, including annealing and wash conditions, are described in ‘Definitions.’

Methods for DNA sequencing are well known in the art and may be used to practice any of the embodiments of the invention. The methods may employ such enzymes as the Klenow fragment of DNA polymerase I, SEQUENASE (US Biochemical, Cleveland Ohio), Taq polymerase (Applied Biosystems), thermostable T7 polymerase (Amersham Biosciences, Piscataway N.J.), or combinations of polymerases and proofreading exonucleases such as those found in the ELONGASE amplification system (Invitrogen, Carlsbad Calif.). Preferably, sequence preparation is automated with machines such as the MICROLAB 2200 liquid transfer system (Hamilton, Reno Nev.), PTC200 thermal cycler (MJ Research, Watertown Mass.) and ABI CATALYST 800 thermal cycler (Applied Biosystems). Sequencing is then carried out using either the ABI 373 or 377 DNA sequencing system (Applied Biosystems), the MEGABACE 1000 DNA sequencing system (Amersham Biosciences), or other systems known in the art. The resulting sequences are analyzed using a variety of algorithms which are well known in the art (Ausubel et al., supra, ch. 7; Meyers, R. A. (1995) Molecular Biology and Biotechnology, Wiley VCH, New York N.Y., pp. 856-853).

The nucleic acids encoding IRAP may be extended utilizing a partial nucleotide sequence and employing various PCR-based methods known in the art to detect upstream sequences, such as promoters and regulatory elements. For example, one method which may be employed, restriction-site PCR, uses universal and nested primers to amplify unknown sequence from genomic DNA within a cloning vector (Sarkar, G. (1993) PCR Methods Applic. 2:318-322). Another method, inverse PCR, uses primers that extend in divergent directions to amplify unknown sequence from a circularized template. The template is derived from restriction fragments comprising a known genomic locus and surrounding sequences (Triglia, T. et al. (1988) Nucleic Acids Res. 16:8186). A third method, capture PCR, involves PCR amplification of DNA fragments adjacent to known sequences in human and yeast artificial chromosome DNA (Lagerstrom, M. et al. (1991) PCR Methods Applic. 1:111-119). In this method, multiple restriction enzyme digestions and ligations may be used to insert an engineered double-stranded sequence into a region of unknown sequence before performing PCR. Other methods which may be used to retrieve unknown sequences are known in the art (Parker, J. D. et al. (1991) Nucleic Acids Res. 19:3055-3060). Additionally, one may use PCR, nested primers, and PROMOTERFINDER libraries (BD Clontech, Palo Alto Calif.) to walk genomic DNA. This procedure avoids the need to screen libraries and is useful in finding intron/exon junctions. For all PCR-based methods, primers may be designed using commercially available software, such as OLIGO 4.06 primer analysis software (National Biosciences, Plymouth Minn.) or another appropriate program, to be about 22 to 30 nucleotides in length, to have a GC content of about 50% or more, and to anneal to the template at temperatures of about 68° C. to 72° C.

When screening for full length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., GENOTYPER and SEQUENCE NAVIGATOR, Applied Biosystems), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotides or fragments thereof which encode IRAP may be cloned in recombinant DNA molecules that direct expression of IRAP, or fragments or functional equivalents thereof, in appropriate host cells. Due to the inherent degeneracy of the genetic code, other polynucleotides which encode substantially the same or a functionally equivalent polypeptides may be produced and used to express IRAP.

The polynucleotides of the invention can be engineered using methods generally known in the art in order to alter IRAP-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

The nucleotides of the present invention may be subjected to DNA shuffling techniques such as MOLECULARBREEDING (Maxygen Inc., Santa Clara Calif.; described in U.S. Pat. No. 5,837,458; Chang, C.-C. et al. (1999) Nat. Biotechnol. 17:793-797; Christians, F. C. et al. (1999) Nat. Biotechnol. 17:259-264; and Crameri, A. et al. (1996) Nat. Biotechnol. 14:315-319) to alter or improve the biological properties of IRAP, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through ‘artificial’ breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

In another embodiment, polynucleotides encoding IRAP may be synthesized, in whole or in part, using one or more chemical methods well known in the art (Caruthers, M. H. et al. (1980) Nucleic Acids Symp. Ser. 7:215-223; Horn, T. et al. (1980) Nucleic Acids Symp. Ser. 7:225-232). Alternatively, IRAP itself or a fragment thereof may be synthesized using chemical methods known in the art. For example, peptide synthesis can be performed using various solution-phase or solid-phase techniques (Creighton, T. (1984) Proteins, Structures and Molecular Properties, WH Freeman, New York N.Y., pp. 55-60; Roberge, J. Y. et al. (1995) Science 269:202-204). Automated synthesis may be achieved using the ABI 431 A peptide synthesizer (Applied Biosystems). Additionally, the amino acid sequence of IRAP, or any part thereof, may be altered during direct synthesis and/or combined with sequences from other proteins, or any part thereof, to produce a variant polypeptide or a polypeptide having a sequence of a naturally occurring polypeptide.

The peptide may be substantially purified by preparative high performance liquid chromatography (Chiez, R. M. and F. Z. Regnier (1990) Methods Enzymol. 182:392-421). The composition of the synthetic peptides may be confirmed by amino acid analysis or by sequencing (Creighton, supra, pp. 28-53).

In order to express a biologically active IRAP, the polynucleotides encoding IRAP or derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotides encoding IRAP. Such elements may vary in their strength and specificity. Specific initiation signals may also be used to achieve more efficient translation of polynucleotides encoding IRAP. Such signals include the ATG initiation codon and adjacent sequences, e.g. the Kozak sequence. In cases where a polynucleotide sequence encoding IRAP and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used (Scharf, D. et al. (1994) Results Probl. Cell Differ. 20:125-162).

Methods which are well known to those skilled in the art may be used to construct expression vectors containing polynucleotides encoding IRAP and appropriate transcriptional and translational control elements. These methods include in vitro recombinant DNA techniques, synthetic techniques, and in vivo genetic recombination (Sambrook and Russell, supra, ch. 1-4, and 8; Ausubel et al., supra, ch. 1, 3, and 15).

A variety of expression vector/host systems may be utilized to contain and express polynucleotides encoding IRAP. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems (Sambrook and Russell, supra; Ausubel et al., supra; Van Heeke, G. and S. M. Schuster (1989) J. Biol. Chem. 264:5503-5509; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; Harrington, J. J. et al. (1997) Nat. Genet. 15:345-355). Expression vectors derived from retroviruses, adenoviruses, or herpes or vaccinia viruses, or from various bacterial plasmids, may be used for delivery of polynucleotides to the targeted organ, tissue, or cell population (Di Nicola, M. et al. (1998) Cancer Gen. Ther. 5:350-356; Yu, M. et al. (1993) Proc. Natl. Acad. Sci. USA 90:6340-6344; Buller, R. M. et al. (1985) Nature 317:813-815; McGregor, D. P. et al. (1994) Mol. Immunol. 31:219-226; Verma, I. M. and N. Somia (1997) Nature 389:239-242). The invention is not limited by the host cell employed.

In bacterial systems, a number of cloning and expression vectors may be selected depending upon the use intended for polynucleotides encoding IRAP. For example, routine cloning, subcloning, and propagation of polynucleotides encoding IRAP can be achieved using a multifunctional E. coli vector such as PBLUESCRIPT (Stratagene, La Jolla Calif.) or PSPORT1 plasmid (Invitrogen). Ligation of polynucleotides encoding IRAP into the vector's multiple cloning site disrupts the lacZ gene, allowing a colorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence (Van Heeke, G. and S. M. Schuster (1989) J. Biol. Chem. 264:5503-5509). When large quantities of IRAP are needed, e.g. for the production of antibodies, vectors which direct high level expression of IRAP may be used. For example, vectors containing the strong, inducible SP6 or T7 bacteriophage promoter may be used.

Yeast expression systems may be used for production of IRAP. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign polynucleotide sequences into the host genome for stable propagation (Ausubel et al., supra; Bitter, G. A. et al. (1987) Methods Enzymol. 153:516-544; Scorer, C. A. et al. (1994) Bio/Technology 12:181-184).

Plant systems may also be used for expression of IRAP. Transcription of polynucleotides encoding IRAP may be driven by viral promoters, e.g., the 35S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters may be used (Coruzzi, G. et al. (1984) EMBO J. 3:1671-1680; Broglie, R. et al. (1984) Science 224:838-843; Winter, J. et al. (1991) Results Probl. Cell Differ. 17:85-105). These constructs can be introduced into plant cells by direct DNA transformation or pathogen-mediated transfection (The McGraw Hill Yearbook of Science and Technology (1992) McGraw Hill, New York N.Y., pp. 191-196).

In mammalian cells, a number of viral-based expression systems may be utilized. In cases where an adenovirus is used as an expression vector, polynucleotides encoding IRAP may be ligated into an adenovirus transcription/translation complex consisting of the late promoter and tripartite leader sequence. Insertion in a non-essential E1 or E3 region of the viral genome may be used to obtain infective virus which expresses IRAP in host cells (Logan, J. and T. Shenk (1984) Proc. Natl. Acad. Sci. USA 81:3655-3659). In addition, transcription enhancers, such as the Rous sarcoma virus (RSV) enhancer, may be used to increase expression in mammalian host cells. SV40 or EBV— based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes (Harrington, J. J. et al. (1997) Nat. Genet. 15:345-355).

For long term production of recombinant proteins in mammalian systems, stable expression of IRAP in cell lines is preferred. For example, polynucleotides encoding IRAP can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk⁻and apr⁻cells, respectively (Wigler, M. et al. (1977) Cell 11:223-232; Lowy, I. et al. (1980) Cell 22:817-823). Also, antimetabolite, antibiotic, or herbicide resistance can be used as the basis for selection. For example, dhfr confers resistance to methotrexate; neo confers resistance to the aminoglycosides neomycin and G-418; and als and pat confer resistance to chlorsulfuron and phosphinotricin acetyltransferase, respectively (Wigler, M. et al. (1980) Proc. Natl. Acad. Sci. USA 77:3567-3570; Colbere-Garapin, F. et al. (1981) J. Mol. Biol. 150:1-14). Additional selectable genes have been described, e.g., trpB and hisD, which alter cellular requirements for metabolites (Hartman, S. C. and R. C. Mulligan (1988) Proc. Natl. Acad. Sci. USA 85:8047-8051). Visible markers, e.g., anthocyanins, green fluorescent proteins (GFP; BD Clontech), β-glucuronidase and its substrate β-glucuronide, or luciferase and its substrate luciferin may be used. These markers can be used not only to identify transformants, but also to quantify the amount of transient or stable protein expression attributable to a specific vector system (Rhodes, Calif. (1995) Methods Mol. Biol. 55:121-131).

Although the presence/absence of marker gene expression suggests that the gene of interest is also present, the presence and expression of the gene may need to be confirmed. For example, if the sequence encoding IRAP is inserted within a marker gene sequence, transformed cells containing polynucleotides encoding IRAP can be identified by the absence of marker gene function. Alternatively, a marker gene can be placed in tandem with a sequence encoding IRAP under the control of a single promoter. Expression of the marker gene in response to induction or selection usually indicates expression of the tandem gene as well.

In general, host cells that contain the polynucleotide encoding IRAP and that express IRAP may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of IRAP using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on IRAP is preferred, but a competitive binding assay may be employed. These and other assays are well known in the art (Hampton, R. et al. (1990) Serological Methods a Laboratory Manual, APS Press, St. Paul Minn., Sect. IV; Coligan, J. E. et al. (1997) Current Protocols in Immunology, Greene Pub. Associates and Wiley-Interscience, New York N.Y.; Pound, J. D. (1998) Immunochemical Protocols, Humana Press, Totowa N.J.).

A wide variety of labels and conjugation techniques are known by those skilled in the art and may be used in various nucleic acid and amino acid assays. Means for producing labeled hybridization or PCR probes for detecting sequences related to polynucleotides encoding IRAP include oligolabeling, nick translation, end-labeling, or PCR amplification using a labeled nucleotide. Alternatively, polynucleotides encoding IRAP, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Amersham Biosciences, Promega (Madison Wis.), and US Biochemical. Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with polynucleotides encoding IRAP may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode IRAP may be designed to contain signal sequences which direct secretion of IRAP through a prokaryotic or eukaryotic cell membrane.

In addition, a host cell strain may be chosen for its ability to modulate expression of the inserted polynucleotides or to process the expressed protein in the desired fashion. Such modifications of the polypeptide include, but are not limited to, acetylation, carboxylation, glycosylation, phosphorylation, lipidation, and acylation. Post-translational processing which cleaves a ‘prepro’ or ‘pro’ form of the protein may also be used to specify protein targeting, folding, and/or activity. Different host cells which have specific cellular machinery and characteristic mechanisms for post-translational activities (e.g., CHO, HeLa, MDCK, HEK293, and WI38) are available from the American Type Culture Collection (ATCC, Manassas Va.) and may be chosen to ensure the correct modification and processing of the foreign protein.

In another embodiment of the invention, natural, modified, or recombinant polynucleotides encoding IRAP may be ligated to a heterologous sequence resulting in translation of a fusion protein in any of the aforementioned host systems. For example, a chimeric IRAP protein containing a heterologous moiety that can be recognized by a commercially available antibody may facilitate the screening of peptide libraries for inhibitors of IRAP activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the IRAP encoding sequence and the heterologous protein sequence, so that IRAP may be cleaved away from the heterologous moiety following purification. Methods for fusion protein expression and purification are discussed in Ausubel et al. (supra, ch. 10 and 16). A variety of commercially available kits may also be used to facilitate expression and purification of fusion proteins.

In another embodiment, synthesis of radiolabeled IRAP may be achieved in vitro using the TNT rabbit reticulocyte lysate or wheat germ extract system (Promega). These systems couple transcription and translation of protein-coding sequences operably associated with the T7, T3, or SP6 promoters. Translation takes place in the presence of a radiolabeled amino acid precursor, for example, ³⁵S-methionine.

IRAP, fragments of IRAP, or variants of IRAP may be used to screen for compounds that specifically bind to IRAP. One or more test compounds may be screened for specific binding to IRAP. In various embodiments, 1, 2, 3, 4, 5, 10, 20, 50, 100, or 200 test compounds can be screened for specific binding to IRAP. Examples of test compounds can include antibodies, anticalins, oligonucleotides, proteins (e.g., ligands or receptors), or small molecules.

In related embodiments, variants of IRAP can be used to screen for binding of test compounds, such as antibodies, to IRAP, a variant of IRAP, or a combination of IRAP and/or one or more variants IRAP. In an embodiment, a variant of IRAP can be used to screen for compounds that bind to a variant of IRAP, but not to IRAP having the exact sequence of a sequence of SEQ ID NO: 1. IRAP variants used to perform such screening can have a range of about 50% to about 99% sequence identity to IRAP, with various embodiments having 60%, 70%, 75%, 80%, 85%, 90%, and 95% sequence identity.

In an embodiment, a compound identified in a screen for specific binding to IRAP can be closely related to the natural ligand of IRAP, e.g., a ligand or fragment thereof, a natural substrate, a structural or functional mimetic, or a natural binding partner (Coligan, J. E. et al. (1991) Current Protocols in Immunology 1(2):Chapter 5). In another embodiment, the compound thus identified can be a natural ligand of a receptor IRAP (Howard, A. D. et al. (2001) Trends Pharmacol. Sci. 22:132-140; Wise, A. et al. (2002) Drug Discovery Today 7:235-246).

In other embodiments, a compound identified in a screen for specific binding to IRAP can be closely related to the natural receptor to which IRAP binds, at least a fragment of the receptor, or a fragment of the receptor including all or a portion of the ligand binding site or binding pocket. For example, the compound may be a receptor for IRAP which is capable of propagating a signal, or a decoy receptor for IRAP which is not capable of propagating a signal (Ashkenazi, A. and V. M. Divit (1999) Curr. Opin. Cell Biol. 11:255-260; Mantovani, A. et al. (2001) Trends Immunol. 22:328-336). The compound can be rationally designed using known techniques. Examples of such techniques include those used to construct the compound etanercept (ENBREL; Amgen Inc., Thousand Oaks Calif.), which is efficacious for treating rheumatoid arthritis in humans. Etanercept is an engineered p75 tumor necrosis factor (TNF) receptor dimer linked to the Fc portion of human IgG₁ (Taylor, P. C. et al. (2001) Curr. Opin. Immunol. 13:611-616).

In one embodiment, two or more antibodies having similar or, alternatively, different specificities can be screened for specific binding to IRAP, fragments of IRAP, or variants of IRAP. The binding specificity of the antibodies thus screened can thereby be selected to identify particular fragments or variants of IRAP. In one embodiment, an antibody can be selected such that its binding specificity allows for preferential identification of specific fragments or variants of IRAP. In another embodiment, an antibody can be selected such that its binding specificity allows for preferential diagnosis of a specific disease or condition having increased, decreased, or otherwise abnormal production of IRAP.

In an embodiment, anticalins can be screened for specific binding to IRAP, fragments of IRAP, or variants of IRAP. Anticalins are ligand-binding proteins that have been constructed based on a lipocalin scaffold (Weiss, G. A. and H. B. Lowman (2000) Chem. Biol. 7:R177-R184; Skerra, A. (2001) J. Biotechnol. 74:257-275). The protein architecture of lipocalins can include a beta-barrel having eight antiparallel beta-strands, which supports four loops at its open end. These loops form the natural ligand-binding site of the lipocalins, a site which can be re-engineered in vitro by amino acid substitutions to impart novel binding specificities. The amino acid substitutions can be made using methods known in the art or described herein, and can include conservative substitutions (e.g., substitutions that do not alter binding specificity) or substitutions that modestly, moderately, or significantly alter binding specificity.

In one embodiment, screening for compounds which specifically bind to, stimulate, or inhibit IRAP involves producing appropriate cells which express IRAP, either as a secreted protein or on the cell membrane. Preferred cells can include cells from mammals, yeast, Drosophila, or E. coli. Cells expressing IRAP or cell membrane fractions which contain IRAP are then contacted with a test compound and binding, stimulation, or inhibition of activity of either IRAP or the compound is analyzed.

An assay may simply test binding of a test compound to the polypeptide, wherein binding is detected by a fluorophore, radioisotope, enzyme conjugate, or other detectable label. For example, the assay may comprise the steps of combining at least one test compound with IRAP, either in solution or affixed to a solid support, and detecting the binding of IRAP to the compound. Alternatively, the assay may detect or measure binding of a test compound in the presence of a labeled competitor. Additionally, the assay may be carried out using cell-free preparations, chemical libraries, or natural product mixtures, and the test compound(s) may be free in solution or affixed to a solid support.

An assay can be used to assess the ability of a compound to bind to its natural ligand and/or to inhibit the binding of its natural ligand to its natural receptors. Examples of such assays include radio-labeling assays such as those described in U.S. Pat. No. 5,914,236 and U.S. Pat. No. 6,372,724. In a related embodiment, one or more amino acid substitutions can be introduced into a polypeptide compound (such as a receptor) to improve or alter its ability to bind to its natural ligands (Matthews, D. J. and J. A. Wells. (1994) Chem. Biol. 1:25-30). In another related embodiment, one or more amino acid substitutions can be introduced into a polypeptide compound (such as a ligand) to improve or alter its ability to bind to its natural receptors (Cunningham, B. C. and J. A. Wells (1991) Proc. Natl. Acad. Sci. USA 88:3407-3411; Lowman, H. B. et al. (1991) J. Biol. Chem. 266:10982-10988).

IRAP, fragments of IRAP, or variants of IRAP may be used to screen for compounds that modulate the activity of IRAP. Such compounds may include agonists, antagonists, or partial or inverse agonists. In one embodiment, an assay is performed under conditions permissive for IRAP activity, wherein IRAP is combined with at least one test compound, and the activity of IRAP in the presence of a test compound is compared with the activity of IRAP in the absence of the test compound. A change in the activity of IRAP in the presence of the test compound is indicative of a compound that modulates the activity of IRAP. Alternatively, a test compound is combined with an in vitro or cell-free system comprising IRAP under conditions suitable for IRAP activity, and the assay is performed. In either of these assays, a test compound which modulates the activity of IRAP may do so indirectly and need not come in direct contact with the test compound. At least one and up to a plurality of test compounds may be screened.

another embodiment, polynucleotides encoding IRAP or their mammalian homologs may be ‘knocked out’ in an animal model system using homologous recombination in embryonic stem (ES) cells. Such techniques are well known in the art and are useful for the generation of animal models of human disease (see, e.g., U.S. Pat. No. 5,175,383 and U.S. Pat. No. 5,767,337). For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (neo; Capecchi, M. R. (1989) Science 244:1288-1292). The vector integrates into the corresponding region of the host genome by homologous recombination. Alternatively, homologous recombination takes place using the Cre-loxP system to knockout a gene of interest in a tissue- or developmental stage-specific manner (Marth, J. D. (1996) Clin. Invest. 97:1999-2002; Wagner, K. U. et al. (1997) Nucleic Acids Res. 25:4323-4330). Transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Transgenic animals thus generated may be tested with potential therapeutic or toxic agents.

Polynucleotides encoding IRAP may also be manipulated in vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al. (1998) Science 282:1145-1147).

Polynucleotides encoding IRAP can also be used to create ‘knockin’ humanized animals (pigs) or transgenic animals (mice or rats) to model human disease. With knockin technology, a region of a polynucleotide encoding IRAP is injected into animal ES cells, and the injected sequence integrates into the animal cell genome. Transformed cells are injected into blastulae, and the blastulae are implanted as described above. Transgenic progeny or inbred lines are studied and treated with potential pharmaceutical agents to obtain information on treatment of a human disease. Alternatively, a mammal inbred to overexpress IRAP, e.g., by secreting IRAP in its milk, may also serve as a convenient source of that protein (Janne, J. et al. (1998) Biotechnol. Annu. Rev. 4:55-74).

Therapeutics

Chemical and structural similarity, e.g., in the context of sequences and motifs, exists between regions of IRAP and immune response associated proteins. In addition, examples of tissues expressing IRAP can be found in Table 6 and can also be found in Example XI. Therefore, IRAP appears to play a role in immune system, neurological, developmental, muscle, and cell proliferative disorders. In the treatment of disorders associated with increased IRAP expression or activity, it is desirable to decrease the expression or activity of IRAP. In the treatment of disorders associated with decreased IRAP expression or activity, it is desirable to increase the expression or activity of IRAP.

Therefore, in one embodiment, IRAP or a fragment or derivative thereof may be administered to a subject to treat or prevent a disorder associated with decreased expression or activity of IRAP. Examples of such disorders include, but are not limited to, an immune system disorder such as acquired immunodeficiency syndrome (AIDS), X-linked agammaglobinemia of Bruton, common variable immunodeficiency (CVI), DiGeorge's syndrome (thymic hypoplasia), thymic dysplasia, isolated IgA deficiency, severe combined immunodeficiency disease (SCID), immunodeficiency with thrombocytopenia and eczema (Wiskott-Aldrich syndrome), Chediak-Higashi syndrome, chronic granulomatous diseases, hereditary angioneurotic edema, immunodeficiency associated with Cushing's disease, Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss; a muscle disorder such as cardiomyopathy, myocarditis, Duchenne's muscular dystrophy, Becker's muscular dystrophy, myotonic dystrophy, central core disease, nemaline myopathy, centronuclear myopathy, lipid myopathy, mitochondrial myopathy, infectious myositis, polymyositis, dermatomyositis, inclusion body myositis, thyrotoxic myopathy, and ethanol myopathy; and a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, colon, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus.

In another embodiment, a vector capable of expressing IRAP or a fragment or derivative thereof may be administered to a subject to treat or prevent a disorder associated with decreased expression or activity of IRAP including, but not limited to, those described above.

In a further embodiment, a composition comprising a substantially purified IRAP in conjunction with a suitable pharmaceutical carrier may be administered to a subject to treat or prevent a disorder associated with decreased expression or activity of IRAP including, but not limited to, those provided above.

In still another embodiment, an agonist which modulates the activity of IRAP may be administered to a subject to treat or prevent a disorder associated with decreased expression or activity of IRAP including, but not limited to, those listed above.

In a further embodiment, an antagonist of IRAP may be administered to a subject to treat or prevent a disorder associated with increased expression or activity of IRAP. Examples of such disorders include, but are not limited to, those immune system, neurological, developmental, muscle, and cell proliferative disorders described above. In one aspect, an antibody which specifically binds IRAP may be used directly as an antagonist or indirectly as a targeting or delivery mechanism for bringing a pharmaceutical agent to cells or tissues which express IRAP.

In an additional embodiment, a vector expressing the complement of the polynucleotide encoding IRAP may be administered to a subject to treat or prevent a disorder associated with increased expression or activity of IRAP including, but not limited to, those described above.

In other embodiments, any protein, agonist, antagonist, antibody, complementary sequence, or vector embodiments may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of IRAP may be produced using methods which are generally known in the art. In particular, purified IRAP may be used to produce antibodies or to screen libraries of pharmaceutical agents to identify those which specifically bind IRAP. Antibodies to IRAP may also be generated using methods that are well known in the art. Such antibodies may include, but are not limited to, polyclonal, monoclonal, chimeric, and single chain antibodies, Fab fragments, and fragments produced by a Fab expression library. In an embodiment, neutralizing antibodies (i.e., those which inhibit dimer formation) can be used therapeutically. Single chain antibodies (e.g., from camels or llamas) may be potent enzyme inhibitors and may have application in the design of peptide mimetics, and in the development of immuno-adsorbents and biosensors (Muyldermans, S. (2001) J. Biotechnol. 74:277-302).

For the production of antibodies, various hosts including goats, rabbits, rats, mice, camels, dromedaries, llamas, humans, and others may be immunized by injection with IRAP or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to IRAP have an amino acid sequence consisting of at least about 5 amino acids, and generally will consist of at least about 10 amino acids. It is also preferable that these oligopeptides, peptides, or fragments are substantially identical to a portion of the amino acid sequence of the natural protein. Short stretches of IRAP amino acids may be fused with those of another protein, such as KLH, and antibodies to the chimeric molecule may be produced.

Monoclonal antibodies to IRAP may be prepared using any technique which provides for the production of antibody molecules by continuous cell lines in culture. These include, but are not limited to, the hybridoma technique, the human B-cell hybridoma technique, and the EBV-hybridoma technique (Kohler, G. et al. (1975) Nature 256:495-497; Kozbor, D. et al. (1985) J. Immunol. Methods 81:31-42; Cote, R. J. et al. (1983) Proc. Natl. Acad. Sci. USA 80:2026-2030; Cole, S. P. et al. (1984) Mol. Cell Biol. 62:109-120).

In addition, techniques developed for the production of ‘chimeric antibodies,’ such as the splicing of mouse antibody genes to human antibody genes to obtain a molecule with appropriate antigen specificity and biological activity, can be used (Morrison, S. L. et al. (1984) Proc. Natl. Acad. Sci. USA 81:6851-6855; Neuberger, M. S. et al. (1984) Nature 312:604-608; Takeda, S. et al. (1985) Nature 314:452-454). Alternatively, techniques described for the production of single chain antibodies may be adapted, using methods known in the art, to produce IRAP-specific single chain antibodies. Antibodies with related specificity, but of distinct idiotypic composition, may be generated by chain shuffling from random combinatorial immunoglobulin libraries (Burton, D. R. (1991) Proc. Natl. Acad. Sci. USA 88:10134-10137).

Antibodies may also be produced by inducing in vivo production in the lymphocyte population or by screening immunoglobulin libraries or panels of highly specific binding reagents as disclosed in the literature (Orlandi, R. et al. (1989) Proc. Natl. Acad. Sci. USA 86:3833-3837; Winter, G. et al. (1991) Nature 349:293-299).

Antibody fragments which contain specific binding sites for IRAP may also be generated. For example, such fragments include, but are not limited to, F(ab′)₂ fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity (Huse, W. D. et al. (1989) Science 246:1275-1281).

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between IRAP and its specific antibody. A two-site, monoclonal-based immunoassay utilizing monoclonal antibodies reactive to two non-interfering IRAP epitopes is generally used, but a competitive binding assay may also be employed (Pound, supra).

Various methods such as Scatchard analysis in conjunction with radioimmunoassay techniques may be used to assess the affinity of antibodies for IRAP. Affinity is expressed as an association constant, Kₐ, which is defined as the molar concentration of IRAP-antibody complex divided by the molar concentrations of free antigen and free antibody under equilibrium conditions. The Kₐ determined for a preparation of polyclonal antibodies, which are heterogeneous in their affinities for multiple IRAP epitopes, represents the average affinity, or avidity, of the antibodies for IRAP. The Kₐ determined for a preparation of monoclonal antibodies, which are monospecific for a particular IRAP epitope, represents a true measure of affinity. High-affinity antibody preparations with Kₐ ranging from about 10⁹ to 10¹² L/mole are preferred for use in immunoassays in which the IRAP-antibody complex must withstand rigorous manipulations. Low-affinity antibody preparations with Kₐ ranging from about 10⁶ to 10⁷ L/mole are preferred for use in immunopurification and similar procedures which ultimately require dissociation of IRAP, preferably in active form, from the antibody (Catty, D. (1988) Antibodies, Volume I: A Practical Approach, IRL Press, Washington D. C. ; Liddell, J. E. and A. Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of IRAP-antibody complexes. Procedures for evaluating antibody specificity, titer, and avidity, and guidelines for antibody quality and usage in various applications, are generally available (Catty, supra; Coligan et al., supra).

In another embodiment of the invention, polynucleotides encoding IRAP, or any fragment or complement thereof, may be used for therapeutic purposes. In one aspect, modifications of gene expression can be achieved by designing complementary sequences or antisense molecules (DNA, RNA, PNA, or modified oligonucleotides) to the coding or regulatory regions of the gene encoding IRAP. Such technology is well known in the art, and antisense oligonucleotides or larger fragments can be designed from various locations along the coding or control regions of sequences encoding IRAP (Agrawal, S., ed. (1996) Antisense Therapeutics, Humana Press, Totawa N.J.).

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein (Slater, J. E. et al. (1 998) J. Allerg Clin. Immunol. 102:469-475; Scanlon, K. J. et al. (1995) FASEB J. 9:1288-1296). Antisense sequences can also be introduced intracellularly through the use of viral vectors, such as retrovirus and adeno-associated virus vectors (Miller, A. D. (1990) Blood 76:271-278; Ausubel et al., supra; Uckert, W. and W. Walther (1994) Pharmacol. Ther. 63:323-347). Other gene delivery mechanisms include liposome-derived systems, artificial viral envelopes, and other systems known in the art (Rossi, J. J. (1995) Br. Med. Bull. 51:217-225; Boado, R. J. et al. (1998) J. Pharm. Sci. 87:1308-1315; Morris, M. C. et al. (1997) Nucleic Acids Res. 25:2730-2736).

In another embodiment of the invention, polynucleotides encoding IRAP may be used for somatic or germline gene therapy. Gene therapy may be performed to (i) correct a genetic deficiency (e.g., in the cases of severe combined immunodeficiency (SCID)-X1 disease characterized by X-linked inheritance (Cavazzana-Calvo, M. et al. (2000) Science 288:669-672), severe combined immunodeficiency syndrome associated with an inherited adenosine deaminase (ADA) deficiency (Blaese, R. M. et al. (1995) Science 270:475-480; Bordignon, C. et al. (1995) Science 270:470-475), cystic fibrosis (Zabner, J. et al. (1993) Cell 75:207-216; Crystal, R. G. et al. (1995) Hum. Gene Therapy 6:643-666; Crystal, R. G. et al. (1995) Hum. Gene Therapy 6:667-703), thalassamias, familial hypercholesterolemia, and hemophilia resulting from Factor VIII or Factor IX deficiencies (Crystal, R. G. (1995) Science 270:404-410; Verma, I. M. and N. Somia (1997) Nature 389:239-242)), (ii) express a conditionally lethal gene product (e.g., in the case of cancers which result from unregulated cell proliferation), or (iii) express a protein which affords protection against intracellular parasites (e.g., against human retroviruses, such as human immunodeficiency virus (HIV) (Baltimore, D. (1988) Nature 335:395-396; Poeschla, E. et al. (1996) Proc. Natl. Acad. Sci. USA 93:11395-11399), hepatitis B or C virus (HBV, HCV); fungal parasites, such as Candida albicans and Paracoccidioides brasiliensis; and protozoan parasites such as Plasmodium falciparum and Trypanosoma cruzi). In the case where a genetic deficiency in IRAP expression or regulation causes disease, the expression of IRAP from an appropriate population of transduced cells may alleviate the clinical manifestations caused by the genetic deficiency.

In a further embodiment of the invention, diseases or disorders caused by deficiencies in IRAP are treated by constructing mammalian expression vectors encoding IRAP and introducing these vectors by mechanical means into IRAP-deficient cells. Mechanical transfer technologies for use with cells in vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and W. F. Anderson (1993) Annu. Rev. Biochem. 62:191-217; Ivics, Z. (1997) Cell 91:501-510; Boulay, J.-L. and H. Recipon (1998) Curr. Opin. Biotechnol. 9:445-450).

Expression vectors that may be effective for the expression of IRAP include, but are not limited to, the PCDNA 3.1, EPITAG, PRCCMV2, PREP, PVAX, PCR2-TOPOTA vectors (Invitrogen, Carlsbad Calif.), PCMV-SCRIPT, PCMV-TAG, PEGSH/PERV (Stratagene, La Jolla Calif.), and PTET-OFF, PTET-ON, PTRE2, PTRE2-LUC, PTK-HYG (BD Clontech, Palo Alto Calif.). IRAP may be expressed using (i) a constitutively active promoter, (e.g., from cytomegalovirus (CMV), Rous sarcoma virus (RSV), SV40 virus, thymidine kinase (TK), or β-actin genes), (ii) an inducible promoter (e.g., the tetracycline-regulated promoter (Gossen, M. and H. Bujard (1992) Proc. Natl. Acad. Sci. USA 89:5547-5551; Gossen, M. et al. (1995) Science 268:1766-1769; Rossi, F. M. V. and H. M. Blau (1998) Curr. Opin. Biotechnol. 9:451-456), commercially available in the T-REX plasmid (Invitrogen)); the ecdysone-inducible promoter (available in the plasmids PVGRXR and PIND; Invitrogen); the FK506/rapamycin inducible promoter; or the RU486/mifepristone inducible promoter (Rossi, F. M. V. and H. M. Blau, supra)), or (iii) a tissue-specific promoter or the native promoter of the endogenous gene encoding IRAP from a normal individual.

Commercially available liposome transformation kits (e.g., the PERFECT LIPID TRANSFECTION KIT, available from Invitrogen) allow one with ordinary skill in the art to deliver polynucleotides to target cells in culture and require minimal effort to optimize experimental parameters. In the alternative, transformation is performed using the calcium phosphate method (Graham, F. L. and A. J. Eb (1973) Virology 52:456-467), or by electroporation (Neumann, E. et al. (1982) EMBO J. 1:841-845). The introduction of DNA to primary cells requires modification of these standardized mammalian transfection protocols.

In another embodiment of the invention, diseases or disorders caused by genetic defects with respect to IRAP expression are treated by constructing a retrovirus vector consisting of (i) the polynucleotide encoding IRAP under the control of an independent promoter or the retrovirus long terminal repeat (LTR) promoter, (ii) appropriate RNA packaging signals, and (iii) a Rev-responsive element (RRE) along with additional retrovirus cis-acting RNA sequences and coding sequences required for efficient vector propagation. Retrovirus vectors (e.g., PFB and PFBNEO) are commercially available (Stratagene) and are based on published data (Riviere, I. et al. (1995) Proc. Natl. Acad. Sci. USA 92:6733-6737), incorporated by reference herein. The vector is propagated in an appropriate vector producing cell line (VPCL) that expresses an envelope gene with a tropism for receptors on the target cells or a promiscuous envelope protein such as VSVg (Armentano, D. et al. (1987) J. Virol. 61:1647-1650; Bender, M. A. et al. (1987) J. Virol. 61:1639-1646; Adam, M. A. and A. D. Miller (1988) J. Virol. 62:3802-3806; Dull, T. et al. (1998) J. Virol. 72:8463-8471; Zufferey, R. et al. (1998) J. Virol. 72:9873-9880). U.S. Pat. No. 5,910,434 to Rigg (‘Method for obtaining retrovirus packaging cell lines producing high transducing efficiency retroviral supernatant’) discloses a method for obtaining retrovirus packaging cell lines and is hereby incorporated by reference. Propagation of retrovirus vectors, transduction of a population of cells (e.g., CD4⁺T-cells), and the return of transduced cells to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M. L. (1997) J. Virol. 71:4707-4716; Ranga, U. et al. (1998) Proc. Natl. Acad. Sci. USA 95:1201-1206; Su, L. (1997) Blood 89:2283-2290).

In an embodiment, an adenovirus-based gene therapy delivery system is used to deliver polynucleotides encoding IRAP to cells which have one or more genetic abnormalities with respect to the expression of IRAP. The construction and packaging of adenovirus-based vectors are well known to those with ordinary skill in the art. Replication defective adenovirus vectors have proven to be versatile for importing genes encoding immunoregulatory proteins into intact islets in the pancreas (Csete, M. E. et al. (1995) Transplantation 27:263-268). Potentially useful adenoviral vectors are described in U.S. Pat. No. 5,707,618 to Armentano (‘Adenovirus vectors for gene therapy’), hereby incorporated by reference. For adenoviral vectors, see also Antinozzi, P. A. et al. (1999; Annu. Rev. Nutr. 19:511-544) and Verma, I. M. and N. Somia (1997; Nature 18:389:239-242).

In another embodiment, a herpes-based, gene therapy delivery system is used to deliver polynucleotides encoding IRAP to target cells which have one or more genetic abnormalities with respect to the expression of IRAP. The use of herpes simplex virus (HSV)-based vectors may be especially valuable for introducing IRAP to cells of the central nervous system, for which HSV has a tropism. The construction and packaging of herpes-based vectors are well known to those with ordinary skill in the art. A replication-competent herpes simplex virus (HSV) type 1-based vector has been used to deliver a reporter gene to the eyes of primates (Liu, X. et al. (1999) Exp. Eve Res. 169:385-395). The construction of a HSV-1 virus vector has also been disclosed in detail in U.S. Pat. No. 5,804,413 to DeLuca (‘Herpes simplex virus strains for gene transfer’), which is hereby incorporated by reference. U.S. Pat. No. 5,804,413 teaches the use of recombinant HSV d92 which consists of a genome containing at least one exogenous gene to be transferred to a cell under the control of the appropriate promoter for purposes including human gene therapy. Also taught by this patent are the construction and use of recombinant HSV strains deleted for ICP4, ICP27 and ICP22. For HSV vectors, see also Goins, W. F. et al. (1999; J. Virol. 73:519-532) and Xu, H. et al. (1994; Dev. Biol. 163:152-161). The manipulation of cloned herpesvirus sequences, the generation of recombinant virus following the transfection of multiple plasmids containing different segments of the large herpesvirus genomes, the growth and propagation of herpesvirus, and the infection of cells with herpesvirus are techniques well known to those of ordinary skill in the art.

In another embodiment, an alphavirus (positive, single-stranded RNA virus) vector is used to deliver polynucleotides encoding IRAP to target cells. The biology of the prototypic alphavirus, Semliki Forest Virus (SFV), has been studied extensively and gene transfer vectors have been based on the SFV genome (Garoff, H. and K.-J. Li (1998) Curr. Opin. Biotechnol. 9:464-469). During alphavirus RNA replication, a subgenomic RNA is generated that normally encodes the viral capsid proteins. This subgenomic RNA replicates to higher levels than the full length genomic RNA, resulting in the overproduction of capsid proteins relative to the viral proteins with enzymatic activity (e.g., protease and polymerase). Similarly, inserting the coding sequence for IRAP into the alphavirus genome in place of the capsid-coding region results in the production of a large number of IRAP-coding RNAs and the synthesis of high levels of IRAP in vector transduced cells. While alphavirus infection is typically associated with cell lysis within a few days, the ability to establish a persistent infection in hamster normal kidney cells (BHK-21) with a variant of Sindbis virus (SIN) indicates that the lytic replication of alphaviruses can be altered to suit the needs of the gene therapy application (Dryga, S. A. et al. (1997) Virology 228:74-83). The wide host range of alphaviruses will allow the introduction of IRAP into a variety of cell types. The specific transduction of a subset of cells in a population may require the sorting of cells prior to transduction. The methods of manipulating infectious cDNA clones of alphaviruses, performing alphavirus cDNA and RNA transfections, and performing alphavirus infections, are well known to those with ordinary skill in the art.

Oligonucleotides derived from the transcription initiation site, e.g., between about positions−10 and +10 from the start site, may also be employed to inhibit gene expression. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) in Huber, B. E. and B. I. Carr, Molecular and Immunologic Approaches, Futura Publishing, Mt. Kisco N.Y., pp. 163-177). A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of RNA molecules encoding IRAP.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA molecules encoding IRAP. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

In other embodiments of the invention, the expression of one or more selected polynucleotides of the present invention can be altered, inhibited, decreased, or silenced using RNA interference (RNAi) or post-transcriptional gene silencing (PTGS) methods known in the art. RNAi is a post-transcriptional mode of gene silencing in which double-stranded RNA (dsRNA) introduced into a targeted cell specifically suppresses the expression of the homologous gene (i.e., the gene bearing the sequence complementary to the dsRNA). This effectively knocks out or substantially reduces the expression of the targeted gene. PTGS can also be accomplished by use of DNA or DNA fragments as well. RNAi methods are described by Fire, A. et al. (1998; Nature 391:806-811) and Gura, T. (2000; Nature 404:804-808). PTGS can also be initiated by introduction of a complementary segment of DNA into the selected tissue using gene delivery and/or viral vector delivery methods described herein or known in the art.

RNAi can be induced in mammalian cells by the use of small interfering RNA also known as siRNA. siRNA are shorter segments of dsRNA (typically about 21 to 23 nucleotides in length) that result in vivo from cleavage of introduced dsRNA by the action of an endogenous ribonuclease. siRNA appear to be the mediators of the RNAi effect in mammals. The most effective siRNAs appear to be 21 nucleotide dsRNAs with 2 nucleotide 3′ overhangs. The use of siRNA for inducing RNAi in mammalian cells is described by Elbashir, S. M. et al. (2001; Nature 411:494-498).

siRNA can be generated indirectly by introduction of dsRNA into the targeted cell. Alternatively, siRNA can be synthesized directly and introduced into a cell by transfection methods and agents described herein or known in the art (such as liposome-mediated transfection, viral vector methods, or other polynucleotide delivery/introductory methods). Suitable siRNAs can be selected by examining a transcript of the target polynucleotide (e.g., mRNA) for nucleotide sequences downstream from the AUG start codon and recording the occurrence of each nucleotide and the 3′ adjacent 19 to 23 nucleotides as potential siRNA target sites, with sequences having a 21 nucleotide length being preferred. Regions to be avoided for target siRNA sites include the 5′ and 3′ untranslated regions (UTRs) and regions near the start codon (within 75 bases), as these may be richer in regulatory protein binding sites. UTR-binding proteins and/or translation initiation complexes may interfere with binding of the siRNP endonuclease complex. The selected target sites for siRNA can then be compared to the appropriate genome database (e.g., human, etc.) using BLAST or other sequence comparison algorithms known in the art. Target sequences with significant homology to other coding sequences can be eliminated from consideration. The selected siRNAs can be produced by chemical synthesis methods known in the art or by in vitro transcription using commercially available methods and kits such as the SILENCER siRNA construction kit (Ambion, Austin Tex.).

In alternative embodiments, long-term gene silencing and/or RNAi effects can be induced in selected tissue using expression vectors that continuously express siRNA. This can be accomplished using expression vectors that are engineered to express hairpin RNAs (shRNAs) using methods known in the art (see, e.g., Brummelkamp, T. R. et al. (2002) Science 296:550-553; and Paddison, P. J. et al. (2002) Genes Dev. 16:948-958). In these and related embodiments, shRNAs can be delivered to target cells using expression vectors known in the art. An example of a suitable expression vector for delivery of siRNA is the PSILENCER1.0-U6 (circular) plasmid (Ambion). Once delivered to the target tissue, shRNAs are processed in vivo into siRNA-like molecules capable of carrying out gene-specific silencing.

In various embodiments, the expression levels of genes targeted by RNAi or PTGS methods can be determined by assays for mRNA and/or protein analysis. Expression levels of the mRNA of a targeted gene can be determined, for example, by northern analysis methods using the NORTHERNMAX-GLY kit (Ambion); by microarray methods; by PCR methods; by real time PCR methods; and by other RNA/polynucleotide assays known in the art or described herein. Expression levels of the protein encoded by the targeted gene can be determined, for example, by microarray methods; by polyacrylamide gel electrophoresis; and by Western analysis using standard techniques known in the art.

An additional embodiment of the invention encompasses a method for screening for a compound which is effective in altering expression of a polynucleotide encoding IRAP. Compounds which may be effective in altering expression of a specific polynucleotide may include, but are not limited to, oligonucleotides, antisense oligonucleotides, triple helix-forming oligonucleotides, transcription factors and other polypeptide transcriptional regulators, and non-macromolecular chemical entities which are capable of interacting with specific polynucleotide sequences. Effective compounds may alter polynucleotide expression by acting as either inhibitors or promoters of polynucleotide expression. Thus, in the treatment of disorders associated with increased IRAP expression or activity, a compound which specifically inhibits expression of the polynucleotide encoding IRAP may be therapeutically useful, and in the treatment of disorders associated with decreased IRAP expression or activity, a compound which specifically promotes expression of the polynucleotide encoding IRAP may be therapeutically useful.

In various embodiments, one or more test compounds may be screened for effectiveness in altering expression of a specific polynucleotide. A test compound may be obtained by any method commonly known in the art, including chemical modification of a compound known to be effective in altering polynucleotide expression; selection from an existing, commercially-available or proprietary library of naturally-occurring or non-natural chemical compounds; rational design of a compound based on chemical and/or structural properties of the target polynucleotide; and selection from a library of chemical compounds created combinatorially or randomly. A sample comprising a polynucleotide encoding IRAP is exposed to at least one test compound thus obtained. The sample may comprise, for example, an intact or permeabilized cell, or an in vitro cell-free or reconstituted biochemical system. Alterations in the expression of a polynucleotide encoding IRAP are assayed by any method commonly known in the art. Typically, the expression of a specific nucleotide is detected by hybridization with a probe having a nucleotide sequence complementary to the sequence of the polynucleotide encoding IRAP. The amount of hybridization may be quantified, thus forming the basis for a comparison of the expression of the polynucleotide both with and without exposure to one or more test compounds. Detection of a change in the expression of a polynucleotide exposed to a test compound indicates that the test compound is effective in altering the expression of the polynucleotide. A screen for a compound effective in altering expression of a specific polynucleotide can be carried out, for example, using a Schizosaccharomyces pombe gene expression system (Atkins, D. et al. (1999) U.S. Pat. No. 5,932,435; Arndt, G. M. et al. (2000) Nucleic Acids Res. 28:E15) or a human cell line such as HeLa cell (Clarke, M. L. et al. (2000) Biochem. Biophys. Res. Commun. 268:8-13). A particular embodiment of the present invention involves screening a combinatorial library of oligonucleotides (such as deoxyribonucleotides, ribonucleotides, peptide nucleic acids, and modified oligonucleotides) for antisense activity against a specific polynucleotide sequence (Bruice, T. W. et al. (1997) U.S. Pat. No. 5,686,242; Bruice, T. W. et al. (2000) U.S. Pat. No. 6,022,691).

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art (Goldman, C. K. et al. (1997) Nat. Biotechnol. 15:462-466).

Any of the therapeutic methods described above may be applied to any subject in need of such therapy, including, for example, mammals such as humans, dogs, cats, cows, horses, rabbits, and monkeys.

An additional embodiment of the invention relates to the administration of a composition which generally comprises an active ingredient formulated with a pharmaceutically acceptable excipient. Excipients may include, for example, sugars, starches, celluloses, gums, and proteins. Various formulations are commonly known and are thoroughly discussed in the latest edition of Remington's Pharmaceutical Sciences (Maack Publishing, Easton Pa.). Such compositions may consist of IRAP, antibodies to IRAP, and mimetics, agonists, antagonists, or inhibitors of IRAP.

In various embodiments, the compositions described herein, such as pharmaceutical compositions, may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means.

Compositions for pulmonary administration may be prepared in liquid or dry powder form. These compositions are generally aerosolized immediately prior to inhalation by the patient. In the case of small molecules (e.g. traditional low molecular weight organic drugs), aerosol delivery of fast-acting formulations is well-known in the art. In the case of macromolecules (e.g. larger peptides and proteins), recent developments in the field of pulmonary delivery via the alveolar region of the lung have enabled the practical delivery of drugs such as insulin to blood circulation (see, e.g., Patton, J. S. et al., U.S. Pat. No. 5,997,848). Pulmonary delivery allows administration without needle injection, and obviates the need for potentially toxic penetration enhancers.

Compositions suitable for use in the invention include compositions wherein the active ingredients are contained in an effective amount to achieve the intended purpose. The determination of an effective dose is well within the capability of those skilled in the art.

Specialized forms of compositions may be prepared for direct intracellular delivery of macromolecules comprising IRAP or fragments thereof. For example, liposome preparations containing a cell-impermeable macromolecule may promote cell fusion and intracellular delivery of the macromolecule. Alternatively, IRAP or a fragment thereof may be joined to a short cationic N-terminal portion from the HIV Tat-1 protein. Fusion proteins thus generated have been found to transduce into the cells of all tissues, including the brain, in a mouse model system (Schwarze, S. R. et al. (1999) Science 285:1569-1572).

For any compound, the therapeutically effective dose can be estimated initially either in cell culture assays, e.g., of neoplastic cells, or in animal models such as mice, rats, rabbits, dogs, monkeys, or pigs. An animal model may also be used to determine the appropriate concentration range and route of administration. Such information can then be used to determine useful doses and routes for administration in humans.

A therapeutically effective dose refers to that amount of active ingredient, for example IRAP or fragments thereof, antibodies of IRAP, and agonists, antagonists or inhibitors of IRAP, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of toxic to therapeutic effects is the therapeutic index, which can be expressed as the LD₅/ED₅₀ ratio. Compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting compositions may be administered every 3 to 4 days, every week, or biweekly depending on the half-life and clearance rate of the particular formulation.

Normal dosage amounts may vary from about 0.1 μg to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Diagnostics

In another embodiment, antibodies which specifically bind IRAP may be used for the diagnosis of disorders characterized by expression of IRAP, or in assays to monitor patients being treated with IRAP or agonists, antagonists, or inhibitors of IRAP. Antibodies useful for diagnostic purposes may be prepared in the same manner as described above for therapeutics. Diagnostic assays for IRAP include methods which utilize the antibody and a label to detect IRAP in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring IRAP, including ELISAs, RIAs, and FACS, are known in the art and provide a basis for diagnosing altered or abnormal levels of IRAP expression. Normal or standard values for IRAP expression are established by combining body fluids or cell extracts taken from normal mammalian subjects, for example, human subjects, with antibodies to IRAP under conditions suitable for complex formation. The amount of standard complex formation may be quantitated by various methods, such as photometric means. Quantities of IRAP expressed in subject, control, and disease samples from biopsied tissues are compared with the standard values. Deviation between standard and subject values establishes the parameters for diagnosing disease.

In another embodiment of the invention, polynucleotides encoding IRAP may be used for diagnostic purposes. The polynucleotides which may be used include oligonucleotides, complementary RNA and DNA molecules, and PNAs. The polynucleotides may be used to detect and quantify gene expression in biopsied tissues in which expression of IRAP may be correlated with disease. The diagnostic assay may be used to determine absence, presence, and excess expression of IRAP, and to monitor regulation of IRAP levels during therapeutic intervention.

In one aspect, hybridization with PCR probes which are capable of detecting polynucleotides, including genomic sequences, encoding IRAP or closely related molecules may be used to identify nucleic acid sequences which encode IRAP. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding IRAP, allelic variants, or related sequences.

Probes may also be used for the detection of related sequences, and may have at least 50% sequence identity to any of the IRAP encoding sequences. The hybridization probes of the subject invention may be DNA or RNA and may be derived from the sequence of SEQ ID NO: 2 or from genomic sequences including promoters, enhancers, and introns of the IRAP gene.

Means for producing specific hybridization probes for polynucleotides encoding IRAP include the cloning of polynucleotides encoding IRAP or IRAP derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or 35S, or by enzymatic labels, such as alkaline phosphatase coupled to the probe via avidin/biotin coupling systems, and the like.

Polynucleotides encoding IRAP may be used for the diagnosis of disorders associated with expression of IRAP. Examples of such disorders include, but are not limited to, an immune system disorder such as acquired immunodeficiency syndrome (AIDS), X-linked agammaglobinemia of Bruton, common variable immunodeficiency (CVI), DiGeorge's syndrome (thymic hypoplasia), thymic dysplasia, isolated IgA deficiency, severe combined immunodeficiency disease (SCID), immunodeficiency with thrombocytopenia and eczema (Wiskott-Aldrich syndrome), Chediak-Higashi syndrome, chronic granulomatous diseases, hereditary angioneurotic edema, immunodeficiency associated with Cushing's disease, Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss; a muscle disorder such as cardiomyopathy, myocarditis, Duchenne's muscular dystrophy, Becker's muscular dystrophy, myotonic dystrophy, central core disease, nemaline myopathy, centronuclear myopathy, lipid myopathy, mitochondrial myopathy, infectious myositis, polymyositis, dermatomyositis, inclusion body myositis, thyrotoxic myopathy, and ethanol myopathy; and a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, colon, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus. Polynucleotides encoding IRAP may be used in Southern or northern analysis, dot blot, or other membrane-based technologies; in PCR technologies; in dipstick, pin, and multiformat ELISA-like assays; and in microarrays utilizing fluids or tissues from patients to detect altered IRAP expression. Such qualitative or quantitative methods are well known in the art.

In a particular embodiment, polynucleotides encoding IRAP may be used in assays that detect the presence of associated disorders, particularly those mentioned above. Polynucleotides complementary to sequences encoding IRAP may be labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of polynucleotides encoding IRAP in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of IRAP, a normal or standard profile for expression is established. This may be accomplished by combining body fluids or cell extracts taken from normal subjects, either animal or human, with a sequence, or a fragment thereof, encoding IRAP, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier, thereby preventing the development or further progression of the cancer.

Additional diagnostic uses for oligonucleotides designed from the sequences encoding IRAP may involve the use of PCR. These oligomers may be chemically synthesized, generated enzymatically, or produced in vitro. Oligomers will preferably contain a fragment of a polynucleotide encoding IRAP, or a fragment of a polynucleotide complementary to the polynucleotide encoding IRAP, and will be employed under optimized conditions for identification of a specific gene or condition. Oligomers may also be employed under less stringent conditions for detection or quantification of closely related DNA or RNA sequences.

In a particular aspect, oligonucleotide primers derived from polynucleotides encoding IRAP may be used to detect single nucleotide polymorphisms (SNPs). SNPs are substitutions, insertions and deletions that are a frequent cause of inherited or acquired genetic disease in humans. Methods of SNP detection include, but are not limited to, single-stranded conformation polymorphism (SSCP) and fluorescent SSCP (fSSCP) methods. In SSCP, oligonucleotide primers derived from polynucleotides encoding IRAP are used to amplify DNA using the polymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In fSSCP, the oligonucleotide primers are fluorescently labeled, which allows detection of the amplimers in high-throughput equipment such as DNA sequencing machines. Additionally, sequence database analysis methods, termed in silico SNP (isSNP), are capable of identifying polymorphisms by comparing the sequence of individual overlapping DNA fragments which assemble into a common consensus sequence. These computer-based methods filter out sequence variations due to laboratory preparation of DNA and sequencing errors using statistical models and automated analyses of DNA sequence chromatograms. In the alternative, SNPs may be detected and characterized by mass spectrometry using, for example, the high throughput MASSARRAY system (Sequenom, Inc., San Diego Calif.).

SNPs may be used to study the genetic basis of human disease. For example, at least 16 common SNPs have been associated with non-insulin-dependent diabetes mellitus. SNPs are also useful for examining differences in disease outcomes in monogenic disorders, such as cystic fibrosis, sickle cell anemia, or chronic granulomatous disease. For example, variants in the mannose-binding lectin, MBL2, have been shown to be correlated with deleterious pulmonary outcomes in cystic fibrosis. SNPs also have utility in pharmacogenomics, the identification of genetic variants that influence a patient's response to a drug, such as life-threatening toxicity. For example, a variation in N-acetyl transferase is associated with a high incidence of peripheral neuropathy in response to the anti-tuberculosis drug isoniazid, while a variation in the core promoter of the ALOX5 gene results in diminished clinical response to treatment with an anti-asthma drug that targets the 5-lipoxygenase pathway. Analysis of the distribution of SNPs in different populations is useful for investigating genetic drift, mutation, recombination, and selection, as well as for tracing the origins of populations and their migrations (Taylor, J. G. et al. (2001) Trends Mol. Med. 7:507-512; Kwok, P.-Y. and Z. Gu (1999) Mol. Med. Today 5:538-543; Nowotny, P. et al. (2001) Curr. Opin. Neurobiol. 11:637-641).

Methods which may also be used to quantify the expression of IRAP include radiolabeling or biotinylating nucleotides, coamplification of a control nucleic acid, and interpolating results from standard curves (Melby, P. C. et al. (1993) J. Immunol. Methods 159:235-244; Duplaa, C. et al. (1993) Anal. Biochem. 212:229-236). The speed of quantitation of multiple samples may be accelerated by running the assay in a high-throughput format where the oligomer or polynucleotide of interest is presented in various dilutions and a spectrophotometric or colorimetric response gives rapid quantitation.

In further embodiments, oligonucleotides or longer fragments derived from any of the polynucleotides described herein may be used as elements on a microarray. The microarray can be used in transcript imaging techniques which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents which are highly effective and display the fewest side effects may be selected for a patient based on his/her pharmacogenomic profile.

In another embodiment, IRAP, fragments of IRAP, or antibodies specific for IRAP may be used as elements on a microarray. The microarray may be used to monitor or measure protein-protein interactions, drug-target interactions, and gene expression profiles, as described above.

A particular embodiment relates to the use of the polynucleotides of the present invention to generate a transcript image of a tissue or cell type. A transcript image represents the global pattern of gene expression by a particular tissue or cell type. Global gene expression patterns are analyzed by quantifying the number of expressed genes and their relative abundance under given conditions and at a given time (Seilhamer et al., ‘Comparative Gene Transcript Analysis,’ U.S. Pat. No. 5,840,484; hereby expressly incorporated by reference herein). Thus a transcript image may be generated by hybridizing the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity.

Transcript images may be generated using transcripts isolated from tissues, cell lines, biopsies, or other biological samples. The transcript image may thus reflect gene expression in vivo, as in the case of a tissue or biopsy sample, or in vitro, as in the case of a cell line.

Transcript images which profile the expression of the polynucleotides of the present invention may also be used in conjunction with in vitro model systems and preclinical evaluation of pharmaceuticals, as well as toxicological testing of industrial and naturally-occurring environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and N. L. Anderson (2000) Toxicol. Lett. 112-113:467-471). If a test compound has a signature similar to that of a compound with known toxicity, it is likely to share those toxic properties. These fingerprints or signatures are most useful and refined when they contain expression information from a large number of genes and gene families. Ideally, a genome-wide measurement of expression provides the highest quality signature. Even genes whose expression is not altered by any tested compounds are important as well, as the levels of expression of these genes are used to normalize the rest of the expression data. The normalization procedure is useful for comparison of expression data after treatment with different compounds. While the assignment of gene function to elements of a toxicant signature aids in interpretation of toxicity mechanisms, knowledge of gene function is not necessary for the statistical matching of signatures which leads to prediction of toxicity (see, for example, Press Release 00-02 from the National Institute of Environmental Health Sciences, released Feb. 29, 2000, available at niehs.nih.gov/oc/news/toxchip.htm). Therefore, it is important and desirable in toxicological screening using toxicant signatures to include all expressed gene sequences.

In an embodiment, the toxicity of a test compound can be assessed by treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention may be quantified. The transcript levels in the treated biological sample are compared with levels in an untreated biological sample. Differences in the transcript levels between the two samples are indicative of a toxic response caused by the test compound in the treated sample.

Another embodiment relates to the use of the polypeptides disclosed herein to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifying the number of expressed proteins and their relative abundance under given conditions and at a given time. A profile of a cell's proteome may thus be generated by separating and analyzing the polypeptides of a particular tissue or cell type. In one embodiment, the separation is achieved using two-dimensional gel electrophoresis, in which proteins from a sample are separated by isoelectric focusing in the first dimension, and then according to molecular weight by sodium dodecyl sulfate slab gel electrophoresis in the second dimension (Steiner and Anderson, supra). The proteins are visualized in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment. The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of interest. In some cases, further sequence data may be obtained for definitive protein identification.

A proteomic profile may also be generated using antibodies specific for IRAP to quantify the levels of IRAP expression. In one embodiment, the antibodies are used as elements on a microarray, and protein expression levels are quantified by contacting the microarray with the sample and detecting the levels of protein bound to each array element (Lueking, A. et al. (1999) Anal. Biochem. 270:103-111; Mendoze, L. G. et al. (1999) Biotechnigues 27:778-788). Detection may be performed by a variety of methods known in the art, for example, by reacting the proteins in the sample with a thiol- or amino-reactive fluorescent compound and detecting the amount of fluorescence bound at each array element.

Toxicant signatures at the proteome level are also useful for toxicological screening, and should be analyzed in parallel with toxicant signatures at the transcript level. There is a poor correlation between transcript and protein abundances for some proteins in some tissues (Anderson, N. L. and J. Seilhamer (1997) Electrophoresis 18:533-537), so proteome toxicant signatures may be useful in the analysis of compounds which do not significantly affect the transcript image, but which alter the proteomic profile. In addition, the analysis of transcripts in body fluids is difficult, due to rapid degradation of mRNA, so proteomic profiling may be more reliable and informative in such cases.

In another embodiment, the toxicity of a test compound is assessed by treating a biological sample containing proteins with the test compound. Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the polypeptides of the present invention.

In another embodiment, the toxicity of a test compound is assessed by treating a biological sample containing proteins with the test compound. Proteins from the biological sample are incubated with antibodies specific to the polypeptides of the present invention. The amount of protein recognized by the antibodies is quantified. The amount of protein in the treated biological sample is compared with the amount in an untreated biological sample. A difference in the amount of protein between the two samples is indicative of a toxic response to the test compound in the treated sample.

Microarrays may be prepared, used, and analyzed using methods known in the art (Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/25116; Shalon, D. et al. (1995) PCT application WO95/35505; Heller, R. A. et al. (1997) Proc. Natl. Acad. Sci. USA 94:2150-2155; Heller, M. J. et al. (1997) U.S. Pat. No.5,605,662). Various types of microarrays are well known and thoroughly described in Schena, M., ed. (1999; DNA Microarrays: A Practical Approach, Oxford University Press, London).

In another embodiment of the invention, nucleic acid sequences encoding IRAP may be used to generate hybridization probes useful in mapping the naturally occurring genomic sequence. Either coding or noncoding sequences may be used, and in some instances, noncoding sequences may be preferable over coding sequences. For example, conservation of a coding sequence among members of a multi-gene family may potentially cause undesired cross hybridization during chromosomal mapping. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries (Harrington, J. J. et al. (1997) Nat. Genet. 15:345-355; Price, C. M. (1993) Blood Rev. 7:127-134; Trask, B. J. (1991) Trends Genet. 7:149-154). Once mapped, the nucleic acid sequences may be used to develop genetic linkage maps, for example, which correlate the inheritance of a disease state with the inheritance of a particular chromosome region or restriction fragment length polymorphism (RFLP) (Lander, E. S. and D. Botstein (1986) Proc. Natl. Acad. Sci. USA 83:7353-7357).

Fluorescent in situ hybridization (FISH) may be correlated with other physical and genetic map data (Heinz-Ulrich, et al. (1995) in Meyers, supra, pp. 965-968). Examples of genetic map data can be found in various scientific journals or at the Online Mendelian Inheritance in Man (OMIM) World Wide Web site. Correlation between the location of the gene encoding IRAP on a physical map and a specific disorder, or a predisposition to a specific disorder, may help define the region of DNA associated with that disorder and thus may further positional cloning efforts.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the exact chromosomal locus is not known. This information is valuable to investigators searching for disease genes using positional cloning or other gene discovery techniques. Once the gene or genes responsible for a disease or syndrome have been crudely localized by genetic linkage to a particular genomic region, e.g., ataxia-telangiectasia to 11q22-23, any sequences mapping to that area may represent associated or regulatory genes for further investigation (Gatti, R. A. et al. (1988) Nature 336:577-580). The nucleotide sequence of the instant invention may also be used to detect differences in the chromosomal location due to translocation, inversion, etc., among normal, carrier, or affected individuals.

In another embodiment of the invention, IRAP, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between IRAP and the agent being tested may be measured.

Another technique for drug screening provides for high throughput screening of compounds having suitable binding affinity to the protein of interest (Geysen, et al. (1984) PCT application WO84/03564). In this method, large numbers of different small test compounds are synthesized on a solid substrate. The test compounds are reacted with IRAP, or fragments thereof, and washed. Bound IRAP is then detected by methods well known in the art. Purified IRAP can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding IRAP specifically compete with a test compound for binding IRAP. In this manner, antibodies can be used to detect the presence of any peptide which shares one or more antigenic determinants with IRAP.

In additional embodiments, the nucleotide sequences which encode IRAP may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent. The following embodiments are, therefore, to be construed as merely illustrative, and not limitative of the remainder of the disclosure in any way whatsoever.

The disclosures of all patents, applications, and publications mentioned above and below, including U.S. Ser. No. 60/407,561, U.S. Ser. No. 60/410,178, U.S. Ser. No. 60/410,571, U.S. Ser. No. 60/419,906, and U.S. Ser. No. 60/421,445, are hereby expressly incorporated by reference.

### EXAMPLES

I. Construction of cDNA Libraries

Inccyte cDNAs are derived from cDNA libraries described in the LIFESEQ database (Incyte, Palo Alto Calif.). Some tissues are homogenized and lysed in guanidinium isothiocyanate, while others are homogenized and lysed in phenol or in a suitable mixture of denaturants, such as TRIZOL (Invitrogen), a monophasic solution of phenol and guanidine isothiocyanate. The resulting lysates are centrifuged over CsCl cushions or extracted with chloroform. RNA is precipitated from the lysates with either isopropanol or sodium acetate and ethanol, or by other routine methods.

Phenol extraction and precipitation of RNA are repeated as necessary to increase RNA purity. In some cases, RNA is treated with DNase. For most libraries, poly(A)+RNA is isolated using oligo d(T)-coupled paramagnetic particles (Promega), OLIGOTEX latex particles (QIAGEN, Chatsworth Calif.), or an OLIGOTEX mRNA purification kit (QIAGEN). Alternatively, RNA is isolated directly from tissue lysates using other RNA isolation kits, e.g., the POLY(A)PURE mRNA purification kit (Ambion, Austin Tex.).

In some cases, Stratagene is provided with RNA and constructs the corresponding cDNA libraries. Otherwise, cDNA is synthesized and cDNA libraries are constructed with the UNIZAP vector system (Stratagene) or SUPERSCRIPT plasmid system (Invitrogen), using the recommended procedures or similar methods known in the art (Ausubel et al., supra, ch. 5). Reverse transcription is initiated using oligo d(T) or random primers. Synthetic oligonucleotide adapters are ligated to double stranded cDNA, and the cDNA is digested with the appropriate restriction enzyme or enzymes. For most libraries, the cDNA is size-selected (300-1000 bp) using SEPHACRYL S1000, SEPHAROSE CL2B, or SEPHAROSE CL4B column chromatography (Amersham Biosciences) or preparative agarose gel electrophoresis. cDNAs are ligated into compatible restriction enzyme sites of the polylinker of a suitable plasmid, e.g., PBLUESCRIPT plasmid (Stratagene), PSPORT1 plasmid (Invitrogen, Carlsbad Calif.), PCDNA2.1 plasmid (Invitrogen), PBK-CMV plasmid (Stratagene), PCR2-TOPOTA plasmid (Invitrogen), PCMV-ICIS plasmid (Stratagene), pIGEN (Incyte, Palo Alto Calif.), pRARE (Incyte), or pINCY (Incyte), or derivatives thereof. Recombinant plasmids are transformed into competent E. coli cells including XL1-Blue, XL1-BlueMRF, or SOLR from Stratagene or DH5α, DH10B, or ElectroMAX DH10B from Invitrogen.

II. Isolation of cDNA Clones

Plasmids obtained as described in Example I are recovered from host cells by in vivo excision using the UNIZAP vector system (Stratagene) or by cell lysis. Plasmids are purified using at least one of the following: a Magic or WIZARD Minipreps DNA purification system (Promega); an AGTC Miniprep purification kit (Edge Biosystems, Gaithersburg Md.); and QIAWELL 8 Plasmid, QIAWELL 8 Plus Plasmid, QIAWELL 8 Ultra Plasmid purification systems or the R.E.A.L. PREP 96 plasmid purification kit from QIAGEN. Following precipitation, plasmids are resuspended in 0.1 ml of distilled water and stored, with or without lyophilization, at 4° C.

Alternatively, plasmid DNA is amplified from host cell lysates using direct link PCR in a high-throughput format (Rao, V. B. (1994) Anal. Biochem. 216:1-14). Host cell lysis and thermal cycling steps are carried out in a single reaction mixture. Samples are processed and stored in 384-well plates, and the concentration of amplified plasmid DNA is quantified fluorometrically using PICOGREEN dye (Molecular Probes, Eugene OR) and a FLUOROSKAN II fluorescence scanner (Labsystems Oy, Helsinki, Finland).

III. Sequencing and Analysis

Incyte cDNA recovered in plasmids as described in Example II are sequenced as follows. Sequencing reactions are processed using standard methods or high-throughput instrumentation such as the ABI CATALYST 800 (Applied Biosystems) thermal cycler or the PTC-200 thermal cycler (MJ Research) in conjunction with the HYDRA microdispenser (Robbins Scientific) or the MICROLAB 2200 (Hamilton) liquid transfer system. cDNA sequencing reactions are prepared using reagents provided by Amersham Biosciences or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides are carried out using the MEGABACE 1000 DNA sequencing system (Amersham Biosciences); the ABI PRISM 373 or 377 sequencing system (Applied Biosystems) in conjunction with standard ABI protocols and base calling software; or other sequence analysis systems known in the art. Reading frames within the cDNA sequences are identified using standard methods (Ausubel et al., supra, ch. 7). Some of the cDNA sequences are selected for extension using the techniques disclosed in Example VIII.

Polynucleotide sequences derived from Incyte cDNAs are validated by removing vector, linker, and poly(A) sequences and by masking ambiguous bases, using algorithms and programs based on BLAST, dynamic programming, and dinucleotide nearest neighbor analysis. The Incyte cDNA sequences or translations thereof are then queried against a selection of public databases such as the GenBank primate, rodent, mammalian, vertebrate, and eukaryote databases, and BLOCKS, PRINTS, DOMO, PRODOM; PROTEOME databases with sequences from Homo sapiens, Rattus norvegicus, Mus musculus, Caenorhabditis elegans, Saccharomyces cerevisiae, Schizosaccharomyces pombe, and Candida albicans (Incyte, Palo Alto Calif.); hidden Markov model (HMM)-based protein family databases such as PFAM, INCY, and TIGRFAM (Haft, D. H. et al. (2001) Nucleic Acids Res. 29:41-43); and HMM-based protein domain databases such as SMART (Schultz, J. et al. (1998) Proc. Natl. Acad. Sci. USA 95:5857-5864; Letunic, I. et al. (2002) Nucleic Acids Res. 30:242-244). (HMM is a probabilistic approach which analyzes consensus primary structures of gene families; see, for example, Eddy, S. R. (1996) Curr. Opin. Struct. Biol. 6:361-365.) The queries are performed using programs based on BLAST, FASTA, BLIMPS, and HMMER. The Incyte cDNA sequences are assembled to produce full length polynucleotide sequences. Alternatively, GenBank cDNAs, GenBank ESTs, stitched sequences, stretched sequences, or Genscan-predicted coding sequences (see Examples IV and V) are used to extend Incyte cDNA assemblages to full length. Assembly is performed using programs based on Phred, Phrap, and Consed, and cDNA assemblages are screened for open reading frames using programs based on GeneMark, BLAST, and FASTA. The full length polynucleotide sequences are translated to derive the corresponding full length polypeptide sequences. Alternatively, a polypeptide may begin at any of the methionine residues of the full length translated polypeptide. Full length polypeptide sequences are subsequently analyzed by querying against databases such as the GenBank protein databases (genpept), SwissProt, the PROTEOME databases, BLOCKS, PRINTS, DOMO, PRODOM, Prosite, hidden Markov model (HMM)-based protein family databases such as PFAM, INCY, and TIGRFAM; and HMM-based protein domain databases such as SMART. Full length polynucleotide sequences are also analyzed using MACDNASIS PRO software (MiraiBio, Alameda Calif.) and LASERGENE software (DNASTAR). Polynucleotide and polypeptide sequence alignments are generated using default parameters specified by the CLUSTAL algorithm as incorporated into the MEGALIGN multisequence alignment program (DNASTAR), which also calculates the percent identity between aligned sequences.

Table 7 summarizes tools, programs, and algorithms used for the analysis and assembly of Incyte cDNA and full length sequences and provides applicable descriptions, references, and threshold parameters. The first column of Table 7 shows the tools, programs, and algorithms used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score or the lower the probability value, the greater the identity between two sequences).

The programs described above for the assembly and analysis of full length polynucleotide and polypeptide sequences are also used to identify polynucleotide sequence fragments from SEQ ID NO: 2. Fragments from about 20 to about 4000 nucleotides which are useful in hybridization and amplification technologies are described in Table 4, column 2.

IV. Identification and Editing of Coding Sequences from Genomic DNA

Putative immune response associated proteins are initially identified by running the Genscan gene identification program against public genomic sequence databases (e.g., gbpri and gbhtg). Genscan is a general-purpose gene identification program which analyzes genomic DNA sequences from a variety of organisms (Burge, C. and S. Karlin (1997) J. Mol. Biol. 268:78-94; Burge, C. and S. Karlin (1998) Curr. Opin. Struct. Biol. 8:346-354). The program concatenates predicted exons to form an assembled cDNA sequence extending from a methionine to a stop codon. The output of Genscan is a FASTA database of polynucleotide and polypeptide sequences. The maximum range of sequence for Genscan to analyze at once is set to 30 kb. To determine which of these Genscan predicted cDNA sequences encode immune response associated proteins, the encoded polypeptides are analyzed by querying against PFAM models for immune response associated proteins. Potential immune response associated proteins are also identified by homology to Incyte cDNA sequences that have been annotated as immune response associated proteins. These selected Genscan-predicted sequences are then compared by BLAST analysis to the genpept and gbpri public databases. Where necessary, the Genscan-predicted sequences are then edited by comparison to the top BLAST hit from genpept to correct errors in the sequence predicted by Genscan, such as extra or omitted exons. BLAST analysis is also used to find any Incyte cDNA or public cDNA coverage of the Genscan-predicted sequences, thus providing evidence for transcription. When Incyte cDNA coverage is available, this information is used to correct or confirm the Genscan predicted sequence. Full length polynucleotide sequences are obtained by assembling Genscan-predicted coding sequences with Incyte cDNA sequences and/or public cDNA sequences using the assembly process described in Example III. Alternatively, full length polynucleotide sequences are derived entirely from edited or unedited Genscan-predicted coding sequences.

V. Assembly of Genomic Sequence Data with cDNA Sequence Data

‘Stitched’ Sequences

Partial cDNA sequences are extended with exons predicted by the Genscan gene identification program described in Example IV. Partial cDNAs assembled as described in Example III are mapped to genomic DNA and parsed into clusters containing related cDNAs and Genscan exon predictions from one or more genomic sequences. Each cluster is analyzed using an algorithm based on graph theory and dynamic programming to integrate cDNA and genomic information, generating possible splice variants that are subsequently confirmed, edited, or extended to create a full length sequence. Sequence intervals in which the entire length of the interval is present on more than one sequence in the cluster are identified, and intervals thus identified are considered to be equivalent by transitivity. For example, if an interval is present on a cDNA and two genomic sequences, then all three intervals are considered to be equivalent. This process allows unrelated but consecutive genomic sequences to be brought together, bridged by cDNA sequence. Intervals thus identified are then ‘stitched’ together by the stitching algorithm in the order that they appear along their parent sequences to generate the longest possible sequence, as well as sequence variants. Linkages between intervals which proceed along one type of parent sequence (cDNA to cDNA or genomic sequence to genomic sequence) are given preference over linkages which change parent type (cDNA to genomic sequence). The resultant stitched sequences are translated and compared by BLAST analysis to the genpept and gbpri public databases. Incorrect exons predicted by Genscan are corrected by comparison to the top BLAST hit from genpept. Sequences are further extended with additional cDNA sequences, or by inspection of genomic DNA, when necessary.

‘Stretched’ Sequences

Partial DNA sequences are extended to full length with an algorithm based on BLAST analysis. First, partial cDNAs assembled as described in Example III are queried against public databases such as the GenBank primate, rodent, mammalian, vertebrate, and eukaryote databases using the BLAST program. The nearest GenBank protein homolog is then compared by BLAST analysis to either Incyte cDNA sequences or GenScan exon predicted sequences described in Example IV. A chimeric protein is generated by using the resultant high-scoring segment pairs (HSPs) to map the translated sequences onto the GenBank protein homolog. Insertions or deletions may occur in the chimeric protein with respect to the original GenBank protein homolog. The GenBank protein homolog, the chimeric protein, or both are used as probes to search for homologous genomic sequences from the public human genome databases. Partial DNA sequences are therefore ‘stretched’ or extended by the addition of homologous genomic sequences. The resultant stretched sequences are examined to determine whether they contain a complete gene.

VI. Chromosomal Mapping of IRAP Encoding Polynucleotides

The sequences used to assemble SEQ ID NO: 2 are compared with sequences from the Incyte LIFESEQ database and public domain databases using BLAST and other implementations of the Smith-Waterman algorithm. Sequences from these databases that matched SEQ ID NO: 2 are assembled into clusters of contiguous and overlapping sequences using assembly algorithms such as Phrap (Table 7). Radiation hybrid and genetic mapping data available from public resources such as the Stanford Human Genome Center (SHGC), Whitehead Institute for Genome Research (WIGR), and Généthon are used to determine if any of the clustered sequences have been previously mapped. Inclusion of a mapped sequence in a cluster results in the assignment of all sequences of that cluster, including its particular SEQ ID NO:, to that map location.

Map locations are represented by ranges, or intervals, of human chromosomes. The map position of an interval, in centiMorgans, is measured relative to the terminus of the chromosome's p-arm. (The centiMorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Généthon which provide boundaries for radiation hybrid markers whose sequences were included in each of the clusters. Human genome maps and other resources available to the public, such as the NCBI ‘GeneMap’99″ World Wide Web site (ncbi.nlm.nih.gov/genemap/), can be employed to determine if previously identified disease genes map within or in proximity to the intervals indicated above.

VII. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound (Sambrook and Russell, supra, ch. 7; Ausubel et al., supra, ch. 4).

Analogous computer techniques applying BLAST are used to search for identical or related molecules in databases such as GenBank or LIFESEQ (Incyte). This analysis is much faster than multiple membrane-based hybridizations. In addition, the sensitivity of the computer search can be modified to determine whether any particular match is categorized as exact or similar. The basis of the search is the product score, which is defined as:

$$\frac{BLAST Score \times Percent Identity}{5 \times minimum \{ length ( Seq .1 ) , length ( Seq .2 ) \}}$$

The product score takes into account both the degree of similarity between two sequences and the length of the sequence match. The product score is a normalized value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP), and −4 for every mismatch. Two sequences may share more than one HSP (separated by gaps). If there is more than one HSP, then the pair with the highest BLAST score is used to calculate the product score. The product score represents a balance between fractional overlap and quality in a BLAST alignment. For example, a product score of 100 is produced only for 100% identity over the entire length of the shorter of the two sequences being compared. A product score of 70 is produced either by 100% identity and 70% overlap at one end, or by 88% identity and 100% overlap at the other. A product score of 50 is produced either by 100% identity and 50% overlap at one end, or 79% identity and 100% overlap.

Alternatively, polynucleotides encoding IRAP are analyzed with respect to the tissue sources from which they are derived. For example, some full length sequences are assembled, at least in part, with overlapping Incyte cDNA sequences (see Example III). Each cDNA sequence is derived from a cDNA library constructed from a human tissue. Each human tissue is classified into one of the following organ/tissue categories: cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract. The number of libraries in each category is counted and divided by the total number of libraries across all categories. Similarly, each human tissue is classified into one of the following disease/condition categories: cancer, cell line, developmental, inflammation, neurological, trauma, cardiovascular, pooled, and other, and the number of libraries in each category is counted and divided by the total number of libraries across all categories. The resulting percentages reflect the tissue- and disease- specific expression of cDNA encoding IRAP. cDNA sequences and cDNA library/tissue information are found in the LIFESEQ database (Incyte, Palo Alto Calif.).

VIII. Extension of IRAP Encoding Polynucleotides

Full length polynucleotides are produced by extension of an appropriate fragment of the full length molecule using oligonucleotide primers designed from this fragment. One primer is synthesized to initiate 5′ extension of the known fragment, and the other primer is synthesized to initiate 3′ extension of the known fragment. The initial primers are designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations is avoided.

Selected human cDNA libraries are used to extend the sequence. If more than one extension is necessary or desired, additional or nested sets of primers are designed.

High fidelity amplification is obtained by PCR using methods well known in the art. PCR is performed in 96-well plates using the PTC-200 thermal cycler (MJ Research, Inc.). The reaction mix contains DNA template, 200 nmol of each primer, reaction buffer containing Mg²⁺, (NH₄)₂SO₄, and 2-mercaptoethanol, Taq DNA polymerase (Amersham Biosciences), ELONGASE enzyme (Invitrogen), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ are as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well is determined by dispensing 100 μl PICOGREEN quantitation reagent (0.25% (v/v) PICOGREEN; Molecular Probes, Eugene Oreg.) dissolved in 1×TE and 0.5 μl of undiluted PCR product into each well of an opaque fluorimeter plate (Corning Costar, Acton Mass.), allowing the DNA to bind to the reagent. The plate is scanned in a Fluoroskan II (Labsystems Oy, Helsinki, Finland) to measure the fluorescence of the sample and to quantify the concentration of DNA. A 5 μl to 10 μl aliquot of the reaction mixture is analyzed by electrophoresis on a 1% agarose gel to determine which reactions are successful in extending the sequence.

The extended nucleotides are desalted and concentrated, transferred to 384-well plates, digested with CviJI cholera virus endonuclease (Molecular Biology Research, Madison Wis.), and sonicated or sheared prior to religation into pUC 18 vector (Amersham Biosciences). For shotgun sequencing, the digested nucleotides are separated on low concentration (0.6 to 0.8%) agarose gels, fragments are excised, and agar digested with Agar ACE (Promega). Extended clones were religated using T4 ligase (New England Biolabs, Beverly Mass.) into pUC 18 vector (Amersham Biosciences), treated with Pfu DNA polymerase (Stratagene) to fill-in restriction site overhangs, and transfected into competent E. coli cells. Transformed cells are selected on antibiotic-containing media, and individual colonies are picked and cultured overnight at 37° C. in 384-well plates in LB/2×carb liquid media.

The cells are lysed, and DNA is amplified by PCR using Taq DNA polymerase (Amersham Biosciences) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: step 2, 3, and 4 repeated 29 times; Step 6: 72° C., 5 min; Step 7: storage at 4° C. DNA is quantified by PICOGREEN reagent (Molecular Probes) as described above. Samples with low DNA recoveries are reamplified using the same conditions as described above. Samples are diluted with 20% dimethysulfoxide (1:2, v/v), and sequenced using DYENAMIC energy transfer sequencing primers and the DYENAMIC DIRECT kit (Amersham Biosciences) or the ABI PRISM BIGDYE Terminator cycle sequencing ready reaction kit (Applied Biosystems).

In like manner, full length polynucleotides are verified using the above procedure or are used to obtain 5′ regulatory sequences using the above procedure along with oligonucleotides designed for such extension, and an appropriate genomic library.

Ix. Identification of Single Nucleotide Polymorphisms in IRAP Encoding Polynucleotides

Common DNA sequence variants known as single nucleotide polymorphisms (SNPs) are identified in SEQ ID NO: 2 using the LIFESEQ database (Incyte). Sequences from the same gene are clustered together and assembled as described in Example III, allowing the identification of all sequence variants in the gene. An algorithm consisting of a series of filters is used to distinguish SNPs from other sequence variants. Preliminary filters remove the majority of basecall errors by requiring a minimum Phred quality score of 15, and remove sequence alignment errors and errors resulting from improper trimming of vector sequences, chimeras, and splice variants. An automated procedure of advanced chromosome analysis is applied to the original chromatogram files in the vicinity of the putative SNP. Clone error filters use statistically generated algorithms to identify errors introduced during laboratory processing, such as those caused by reverse transcriptase, polymerase, or somatic mutation. Clustering error filters use statistically generated algorithms to identify errors resulting from clustering of close homologs or pseudogenes, or due to contamination by non-human sequences. A final set of filters removes duplicates and SNPs found in immunoglobulins or T-cell receptors.

Certain SNPs are selected for further characterization by mass spectrometry using the high throughput MASSARRAY system (Sequenom, Inc.) to analyze allele frequencies at the SNP sites in four different human populations. The Caucasian population comprises 92 individuals (46 male, 46 female), including 83 from Utah, four French, three Venezualan, and two Amish individuals. The African population comprises 194 individuals (97 male, 97 female), all African Americans. The Hispanic population comprises 324 individuals (162 male, 162 female), all Mexican Hispanic. The Asian population comprises 126 individuals (64 male, 62 female) with a reported parental breakdown of 43% Chinese, 31% Japanese, 13% Korean, 5% Vietnamese, and 8% other Asian. Allele frequencies are first analyzed in the Caucasian population; in some cases those SNPs which show no allelic variance in this population are not further tested in the other three populations.

X. Labeling and Use of Individual Hybridization Probes

Hybridization probes derived from SEQ ID NO: 2 are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 software (National Biosciences) and labeled by combining 50 pmol of each oligomer, 250 μCi of [γ−³²P] adenosine triphosphate (Amersham Biosciences), and T4 polynucleotide kinase (DuPont NEN, Boston Mass.). The labeled oligonucleotides are substantially purified using a SEPHADEX G-25 superfine size exclusion dextran bead column (Amersham Biosciences). An aliquot containing 10⁷ counts per minute of the labeled probe is used in a typical membrane-based hybridization analysis of human genomic DNA digested with one of the following endonucleases: Ase I, Bgl II, Eco RI, Pst I, Xba I, or Pvu II (DuPont NEN).

The DNA from each digest is fractionated on a 0.7% agarose gel and transferred to nylon membranes (Nytran Plus, Schleicher & Schuell, Durham N.H.). Hybridization is carried out for 16 hours at 40° C. To remove nonspecific signals, blots are sequentially washed at room temperature under conditions of up to, for example, 0.1× saline sodium citrate and 0.5% sodium dodecyl sulfate. Hybridization patterns are visualized using autoradiography or an alternative imaging means and compared.

XI. Microarrays

The linkage or synthesis of array elements upon a microarray can be achieved utilizing photolithography, piezoelectric printing (ink-jet printing; see, e.g., Baldeschweiler et al., supra), mechanical microspotting technologies, and derivatives thereof. The substrate in each of the aforementioned technologies should be uniform and solid with a non-porous surface (Schena, M., ed. (1999) DNA Microarrays: A Practical Approach, Oxford University Press, London). Suggested substrates include silicon, silica, glass slides, glass chips, and silicon wafers. Alternatively, a procedure analogous to a dot or slot blot may also be used to arrange and link elements to the surface of a substrate using thermal, UV, chemical, or mechanical bonding procedures. A typical array may be produced using available methods and machines well known to those of ordinary skill in the art and may contain any appropriate number of elements (Schena, M. et al. (1995) Science 270:467-470; Shalon, D. et al. (1996) Genome Res. 6:639-645; Marshall, A. and J. Hodgson (1998) Nat. Biotechnol. 16:27-31).

Full length cDNAs, Expressed Sequence Tags (ESTs), or fragments or oligomers thereof may comprise the elements of the microarray. Fragments or oligomers suitable for hybridization can be selected using software well known in the art such as LASERGENE software (DNASTAR). The array elements are hybridized with polynucleotides in a biological sample. The polynucleotides in the biological sample are conjugated to a fluorescent label or other molecular tag for ease of detection. After hybridization, nonhybridized nucleotides from the biological sample are removed, and a fluorescence scanner is used to detect hybridization at each array element. Alternatively, laser desorbtion and mass spectrometry may be used for detection of hybridization. The degree of complementarity and the relative abundance of each polynucleotide which hybridizes to an element on the microarray may be assessed. In one embodiment, microarray preparation and usage is described in detail below.

Tissue or Cell Sample Preparation

Total RNA is isolated from tissue samples using the guanidinium thiocyanate method and poly(A)⁺RNA is purified using the oligo-(dT) cellulose method. Each poly(A)⁺RNA sample is reverse transcribed using MMLV reverse-transcriptase, 0.05 pg/μl oligo-(dT) primer (21 mer), 1×first strand buffer, 0.03 units/μl RNase inhibitor, 500 μM dATP, 500 μM dGTP, 500 μM dTTP, 40 μM dCTP, 40 μM dCTP-Cy3 (BDS) or dCTP-Cy5 (Amersham Biosciences). The reverse transcription reaction is performed in a 25 ml volume containing 200 ng poly(A)⁺RNA with GEMBRIGHT kits (Incyte). Specific control poly(A)⁺RNAs are synthesized by in vitro transcription from non-coding yeast genomic DNA. After incubation at 37° C. for 2 hr, each reaction sample (one with Cy3 and another with Cy5 labeling) is treated with 2.5 ml of 0.5M sodium hydroxide and incubated for 20 minutes at 85° C. to the stop the reaction and degrade the RNA. Samples are purified using two successive CHROMA SPIN 30 gel filtration spin columns (BD Clontech, Palo Alto Calif.) and after combining, both reaction samples are ethanol precipitated using 1 ml of glycogen (1 mg/ml), 60 ml sodium acetate, and 300 ml of 100% ethanol. The sample is then dried to completion using a SpeedVAC (Savant Instruments Inc., Holbrook N.Y.) and resuspended in 14 μl 5×SSC/0.2% SDS.

Microarray Preparation

Sequences of the present invention are used to generate array elements. Each array element is amplified from bacterial cells containing vectors with cloned cDNA inserts. PCR amplification uses primers complementary to the vector sequences flanking the cDNA insert. Array elements are amplified in thirty cycles of PCR from an initial quantity of 1-2 ng to a final quantity greater than 5 μg. Amplified array elements are then purified using SEPHACRYL-400 (Amersham Biosciences).

Purified array elements are immobilized on polymer-coated glass slides. Glass microscope slides (Corning) are cleaned by ultrasound in 0.1% SDS and acetone, with extensive distilled water washes between and after treatments. Glass slides are etched in 4% hydrofluoric acid (VWR Scientific Products Corporation (VWR), West Chester Pa.), washed extensively in distilled water, and coated with 0.05% aminopropyl silane (Sigma-Aldrich, St. Louis Mo.) in 95% ethanol. Coated slides are cured in a 110° C. oven.

Array elements are applied to the coated glass substrate using a procedure described in U.S. Pat. No. 5,807,522, incorporated herein by reference. 1 μl of the array element DNA, at an average concentration of 100 ng/μl, is loaded into the open capillary printing element by a high-speed robotic apparatus. The apparatus then deposits about 5 nl of array element sample per slide.

Microarrays are UV-crosslinked using a STRATALINKER UV-crosslinker (Stratagene). Microarrays are washed at room temperature once in 0.2% SDS and three times in distilled water. Non-specific binding sites are blocked by incubation of microarrays in 0.2% casein in phosphate buffered saline (PBS) (Tropix, Inc., Bedford Mass.) for 30 minutes at 60° C. followed by washes in 0.2% SDS and distilled water as before.

Hybridization

Hybridization reactions contain 9 μl of sample mixture consisting of 0.2 μg each of Cy3 and Cy5 labeled cDNA synthesis products in 5×SSC., 0.2% SDS hybridization buffer. The sample mixture is heated to 65° C. for 5 minutes and is aliquoted onto the microarray surface and covered with an 1.8 cm² coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hours at 60° C. The arrays are washed for 10 min at 45° C. in a first wash buffer (1×SSC, 0.1% SDS), three times for 10 minutes each at 45° C. in a second wash buffer (0.1×SSC), and dried.

Detection

Reporter-labeled hybridization complexes are detected with a microscope equipped with an Innova 70 mixed gas 10 W laser (Coherent, Inc., Santa Clara Calif.) capable of generating spectral lines at 488 nm for excitation of Cy3 and at 632 nm for excitation of Cy5. The excitation laser light is focused on the array using a 20× microscope objective (Nikon, Inc., Melville N.Y.). The slide containing the array is placed on a computer-controlled X-Y stage on the microscope and raster-scanned past the objective. The 1.8 cm×1.8 cm array used in the present example is scanned with a resolution of 20 micrometers.

In two separate scans, a mixed gas multiline laser excites the two fluorophores sequentially. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the sample mixture at a known concentration. A specific location on the array contains a complementary DNA sequence, allowing the intensity of the signal at that location to be correlated with a weight ratio of hybridizing species of 1:100,000. When two samples from different sources (e.g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit RTI-835H analog-to-digital (A/D) conversion board (Analog Devices, Inc., Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each fluorophore's emission spectrum.

A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS gene expression analysis program (Incyte). Array elements that exhibit at least about a two-fold change in expression, a signal-to-background ratio of at least about 2.5, and an element spot size of at least about 40%, are considered to be differentially expressed.

Expression

In one example, SEQ ID NO: 2 was differentially expressed in peripheral blood mononuclear cells (PBMCs), as determined by microarray technology. PBMCs were collected from the blood of healthy volunteer donors, some of which were pooled, using standard gradient separation. The PBMCs were placed in culture for 2 hours with a number of different cytokines or growth factors, and for various times with bacterial toxins. These included: TNF-α, a protein that plays a critical role in mediation of the inflammatory response and in mediation of resistance to infections and tumor growth; interleukin-1-beta (IL-1β), a cytokine associated with acute inflammatory responses which is generally considered the prototypical pro-inflammatory cytokine; LIF (leukemia inhibitory factor), recognized to be a pleiotropic factor with multiple effects on both hematopoietic and non-hematopoietic cells; leptin, which is involved in body weight regulation, hematopoiesis, and reproduction and interacts with receptors that are expressed in at least four isoforms in very primitive hematopoietic cell populations and in a variety of lymphohematopoietic cell lines; LPS, bacterial endotoxins that are expressed on the outer membrane of gram-negative bacteria; and Staphylococcal exotoxins such as staphlococcal exotoxin B (SEB), which specifically activate human T cells expressing an appropriate TCR-Vβ chain. In the presence of TNF-α (10 ng/ml), expression levels of SEQ ID NO: 2 increased at least 2-fold in PBMCs from specific donors or pooled sets of PBMCs, when compared to untreated PBMCs. In the presence of 10 ng/ml of IL-1β, expression of SEQ ID NO: 2 increased at least 2.5-fold, when examined in pooled PBMCs and compared to untreated cells. Additionally, culture of pooled PBMCs with either LIF (20 ng/ml) or leptin (1 μg/ml) for 2 hours resulted in at least a 2-fold increase in SEQ ID NO: 2 expression, when compared to levels in untreated cells. Upon treatment with LPS (1 μg/ml), an increase in SEQ ID NO: 2 expression was detected in PBMCs from various donors, ranging from 2- to 6-fold after treatment times that varied between donors, but generally were from 1 to 4 hours in length. SEB treatment of PBMCs (1 ng/ml) also resulted in a 2- to 9-fold increase in SEQ ID NO: 2 expression, compared to untreated cells, which peaked between 2 hours and 3 days of treatment, depending on the donor or pool of cells utilized. Therefore, in various embodiments, SEQ ID NO: 2 can be used for one or more of the following: i) monitoring treatment of immune disorders and related diseases and conditions, ii) diagnostic assays for immune disorders and related diseases and conditions, and iii) developing therapeutics and/or other treatments for immune disorders and related diseases and conditions.

In another example, SEQ ID NO: 2 expression was increased in various vascular endothelial cells treated with TNF-α, IFN-γ, and/or IL-1β, as determined by microarray expression analysis. Endothelial cell models are important for studying both physiological and pathophysiological conditions involving activated vascular endothelium, which include vascular tone regulation, coagulation and thrombosis, atherosclerosis, and inflammation. HUVECs (human umbilical vein endothelial cells) were treated with 10 ng/ml each of IL-1β and TNF-α for 24 hours, or with IL-1β alone, and expression of SEQ ID NO: 2 was increased at least 5-fold compared to untreated HUVECs. HCAECs (human coronary artery endothelial cells) treated for 1 hour with 10 ng/ml TNF-α responded with an at least 2-fold increase in SEQ ID NO: 2 expression, compared to untreated HCAECs. In another set of experiments, HCAECs were activated by treatment with 10 ng/ml each of IL-1β and TNF-α for 24 hours, or left untreated. Expression levels of SEQ ID NO: 2 increased between 11.1-fold and 14.7-fold in the treated cells. Additionally, ECV304 cells, a human endothelial cell line, treated with 10 ng/ml each of TNF-α and IFN-γ showed an increase of at least 2-fold in SEQ ID NO: 2 expression from 40 minutes to 24 hours, compared to ECV304 cells treated with IFN-γ alone. In another experiment, ECV304 cells treated with TNF-α alone (10 ng/ml) showed at least a 2.5-fold increase in SEQ ID NO: 2 expression at 40 minutes, which increased to up to 8-fold after 48 hours, in comparison to untreated ECV304 cells. Also, HAECs (human aortic endothelial cells) treated with TNF-α (10 ng/ml) differentially expressed SEQ ID NO: 2 at least 2-fold when compared to untreated HAECs. Therefore, in various embodiments, SEQ ID NO: 2 can be used for one or more of the following: i) monitoring treatment of vascular conditions involving acute or chronic inflammation such as atherosclerosis and chronic arterial hypertension, ii) diagnostic assays for vascular conditions involving acute or chronic inflammation such as atherosclerosis and chronic arterial hypertension, and iii) developing therapeutics and/or other treatments for vascular conditions involving acute or chronic inflammation such as atherosclerosis and chronic arterial hypertension.

In another example, SEQ ID NO: 2 showed differential expression in smooth muscle cells, as determined by microarray expression analysis. Cells such as human coronary artery smooth muscle cells (CASMCs, isolated from the intermediate muscular layer of a coronary artery), PASMCs (pulmonary artery smooth muscle cells), and UASMCs (human umbilical artery smooth muscle cells) serve as important models for studying not only the role of vascular smooth muscle cells in regulation of vascular tone and oxygen supply to tissues, but also their behavior under inflammatory conditions that lead to atherosclerosis and restenosis. CASMCs, PASMCs, and UASMCs were treated for various time periods with TNF-α (10 ng/ml), and the expression levels of SEQ ID NO: 2 were assessed with respect to the levels in untreated cells. In CASMCs, SEQ ID NO: 2 expression increased at least 2-fold after 1 hour of treatment, and remained elevated through 2.3 days. In PASMCs, SEQ ID NO: 2 expression increased at least 2-fold after only 1 hour of TNF-α treatment, and remained elevated through 24 hours. In UASMCs, expression of SEQ ID NO: 2 increased at least 2-fold after 24 hours of TNF-α treatment, compared to untreated UASMCs. In addition, CASMCs treated with both TNF-α and IL-1β at 10 ng/ml each for 24 hours showed at least a 2-fold increase, and up to a 9.2-fold increase in SEQ ID NO: 2 expression levels, compared to control, untreated cells. Therefore, in various embodiments, SEQ ID NO: 2 can be used for one or more of the following: i) monitoring treatment of cardiovascular and other related disorders, ii) diagnostic assays for cardiovascular and other related disorders, and iii) developing therapeutics and/or other treatments for cardiovascular and other related disorders.

In another example, SEQ ID NO: 2 expression was differentially expressed in lung tumor tissue samples, as determined by microarray expression analysis. Tissue was isolated from lung tumors, both adenocarcinomas and squamous cell carcinomas, from various donors, and gene expression levels compared in these samples with normal lung tissue isolated from the same donors. In one set of samples, SEQ ID NO: 2 expression was decreased by at least 3-fold in adenocarcinoma tumor tissue, when compared to matched normal tissue from the same donor. In another set of samples, SEQ ID NO: 2 gene expression levels were decreased by at least 2.5-fold in squamous cell carcinoma lung tumor tissue, when compared to levels detected in matched normal lung tissue from the same donor. Therefore, in various embodiments, SEQ ID NO: 2 can be used for one or more of the following: i) monitoring treatment of lung cancer, ii) diagnostic assays for lung cancer, and iii) developing therapeutics and/or other treatments for lung cancer.

In another example, SEQ ID NO: 2 gene expression levels were analyzed by microarray expression analysis in fibroblasts isolated from patients with Tangier disease (TD). Human fibroblasts were obtained from skin explants from both normal subjects and two patients with homozygous TD. Cell lines were immortalized by transfection with human papillomavirus 16 genes E6 and E7 and a neomycin resistance selectable marker. In addition, both types of cells were cultured in the presence of cholesterol and compared with the same cell type cultured in the absence of cholesterol. TD-derived cells are shown to be deficient in an assay of apoA-I-mediated tritiated cholesterol efflux. Expression of SEQ ID NO: 2 was increased at least 11-fold in fibroblasts from TD patients, when compared to control normal fibroblasts. Similar results were seen when both TD and normal fibroblasts were treated with LDL. Therefore, in various embodiments, SEQ ID NO: 2 can be used for one or more of the following: i) monitoring treatment of Tangier disease, ii) diagnostic assays for Tangier disease, and iii) developing therapeutics and/or other treatments for Tangier disease.

In yet another example, differential expression of SEQ ID NO: 2 was detected in preadipocytes cultured in differentiation media, as determined by microarray expression analysis. Body mass index or BMI is a measurement based on weight and height used to determine whether an individual is of normal weight (18.5-24.9), overweight (25-29.9), or obese (greater than 30). Preadipocytes isolated from an obese (BMI=32.47), but otherwise healthy donor were cultured for various duration in differentiation media containing insulin and PPAR-γ agonist. After 48 hours, 96 hours, and 1.1 weeks of treatment, the expression level of SEQ ID NO: 2 was decreased at least 2-fold, when compared to levels seen in untreated preadipocytes. When preadipocytes isolated from a donor of normal weight (BMI=23.59), SEQ ID NO: 2 expression levels did not changes significantly. Therefore, in various embodiments, SEQ ID NO: 2 can be used for one or more of the following: i) monitoring treatment of obesity, diabetes, and other related conditions, ii) diagnostic assays for obesity, diabetes, and other related conditions, and iii) developing therapeutics and/or other treatments for obesity, diabetes, and other related conditions.

XII. Complementary Polynucleotides

Sequences complementary to the IRAP-encoding sequences, or any parts thereof, are used to detect, decrease, or inhibit expression of naturally occurring IRAP. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of IRAP. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the IRAP-encoding transcript.

XIII. Expression of IRAP

Expression and purification of IRAP is achieved using bacterial or virus-based expression systems. For expression of IRAP in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express IRAP upon induction with isopropyl beta-D-thiogalactopyranoside (IPTG). Expression of IRAP in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding IRAP by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera frugiperda (Sf9) insect cells in most cases, or human hepatocytes, in some cases. Infection of the latter requires additional genetic modifications to baculovirus (Engelhard, E. K. et al. (1994) Proc. Natl. Acad. Sci. USA 91:3224-3227; Sandig, V. et al. (1996) Hum. Gene Ther. 7:1937-1945).

In most expression systems, IRAP is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Amersham Biosciences). Following purification, the GST moiety can be proteolytically cleaved from IRAP at specifically engineered sites. FLAG, an 8-amino acid peptide, enables immunoaffinity purification using commercially available monoclonal and polyclonal anti-FLAG antibodies (Eastman Kodak). 6-His, a stretch of six consecutive histidine residues, enables purification on metal-chelate resins (QIAGEN). Methods for protein expression and purification are discussed in Ausubel et al. (supra, ch. 10 and 16). Purified IRAP obtained by these methods can be used directly in the assays shown in Examples XVII and XVIII, where applicable.

XIV. Functional Assays

IRAP function is assessed by expressing the sequences encoding IRAP at physiologically elevated levels in mammalian cell culture systems. cDNA is subcloned into a mammalian expression vector containing a strong promoter that drives high levels of cDNA expression. Vectors of choice include PCMV SPORT plasmid (Invitrogen, Carlsbad Calif.) and PCR3.1 plasmid (Invitrogen), both of which contain the cytomegalovirus promoter. 5-10 μg of recombinant vector are transiently transfected into a human cell line, for example, an endothelial or hematopoietic cell line, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; BD Clontech), CD64, or a CD64-GFP fusion protein. Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994; Flow Cytometry, Oxford, New York N.Y.).

The influence of IRAP on gene expression can be assessed using highly purified populations of cells transfected with sequences encoding IRAP and either CD64 or CD64-GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Lake Success N.Y.). mRNA can be purified from the cells using methods well known by those of skill in the art. Expression of mRNA encoding IRAP and other genes of interest can be analyzed by northern analysis or microarray techniques.

XV. Production of IRAP Specific Antibodies

IRAP substantially purified using polyacrylamide gel electrophoresis (PAGE; see, e.g., Harrington, M. G. (1990) Methods Enzymol. 182:488-495), or other purification techniques, is used to immunize animals (e.g., rabbits, mice, etc.) and to produce antibodies using standard protocols.

Alternatively, the IRAP amino acid sequence is analyzed using LASERGENE software (DNASTAR) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art (Ausubel et al., supra, ch. 11).

Typically, oligopeptides of about 15 residues in length are synthesized using an ABI 431A peptide synthesizer (Applied Biosystems) using FMOC chemistry and coupled to KLH (Sigma-Aldrich, St. Louis Mo.) by reaction with N-maleimidobenzoyl-N-hydroxysuccinimide ester (MBS) to increase immunogenicity (Ausubel et al., supra). Rabbits are immunized with the oligopeptide-KLH complex in complete Freund's adjuvant. Resulting antisera are tested for antipeptide and anti-IRAP activity by, for example, binding the peptide or IRAP to a substrate, blocking with 1% BSA, reacting with rabbit antisera, washing, and reacting with radio-iodinated goat anti-rabbit IgG.

XVI. Purification of Naturally Occurring IRAP Using Specific Antibodies

Naturally occurring or recombinant IRAP is substantially purified by immunoaffinity chromatography using antibodies specific for IRAP. An immunoaffinity column is constructed by covalently coupling anti-IRAP antibody to an activated chromatographic resin, such as CNBr-activated SEPHAROSE (Amersham Biosciences). After the coupling, the resin is blocked and washed according to the manufacturer's instructions.

Media containing IRAP are passed over the immunoaffinity column, and the column is washed under conditions that allow the preferential absorbance of IRAP (e.g., high ionic strength buffers in the presence of detergent). The column is eluted under conditions that disrupt antibody/IRAP binding (e.g., a buffer of pH 2 to pH 3, or a high concentration of a chaotrope, such as urea or thiocyanate ion), and IRAP is collected.

XVII. Identification of Molecules Which Interact with IRAP

IRAP, or biologically active fragments thereof, are labeled with ¹²⁵I Bolton-Hunter reagent (Bolton, A. E. and W. M. Hunter (1973) Biochem. J. 133:529-539). Candidate molecules previously arrayed in the wells of a multi-well plate are incubated with the labeled IRAP, washed, and any wells with labeled IRAP complex are assayed. Data obtained using different concentrations of IRAP are used to calculate values for the number, affinity, and association of IRAP with the candidate molecules.

Alternatively, molecules interacting with IRAP are analyzed using the yeast two-hybrid system as described in Fields, S. and O. Song (1989; Nature 340:245-246), or using commercially available kits based on the two-hybrid system, such as the MATCHMAKER system (BD Clontech).

IRAP may also be used in the PATHCALLING process (CuraGen Corp., New Haven Conn.) which employs the yeast two-hybrid system in a high-throughput manner to determine all interactions between the proteins encoded by two large libraries of genes (Nandabalan, K. et al. (2000) U.S. Pat. No. 6,057,101).

XVIII. Demonstration of IRAP Activity

An assay for IRAP activity measures the ability of IRAP to recognize and precipitate antigens from serum. This activity can be measured by the quantitative precipitin reaction (Golub, E. S. et al. (1987) Immunology: A Synthesis, Sinauer Associates, Sunderland, Mass., pages 113-115). IRAP is isotopically labeled using methods known in the art. Various serum concentrations are added to constant amounts of labeled IRAP. IRAP-antigen complexes precipitate out of solution and are collected by centrifugation. The amount of precipitable IRAP-antigen complex is proportional to the amount of radioisotope detected in the precipitate. The amount of precipitable IRAP-antigen complex is plotted against the serum concentration. For various serum concentrations, a characteristic precipitin curve is obtained, in which the amount of precipitable IRAP-antigen complex initially increases proportionately with increasing serum concentration, peaks at the equivalence point, and then decreases proportionately with further increases in serum concentration. Thus, the amount of precipitable IRAP-antigen complex is a measure of IRAP activity which is characterized by sensitivity to both limiting and excess quantities of antigen.

Alternatively, an assay for IRAP activity measures the expression of IRAP on the cell surface. cDNA encoding IRAP is transfected into a non-leukocytic cell line. Cell surface proteins are labeled with biotin (de la Fuente, M. A. et al. (1997) Blood 90:2398-2405). Immunoprecipitations are performed using IRAP-specific antibodies, and immunoprecipitated samples are analyzed using SDS- PAGE and immunoblotting techniques. The ratio of labeled immunoprecipitant to unlabeled immunoprecipitant is proportional to the amount of IRAP expressed on the cell surface.

Alternatively, an assay for IRAP activity measures the amount of cell aggregation induced by overexpression of IRAP. In this assay, cultured cells such as NIH3T3 are transfected with cDNA encoding IRAP contained within a suitable mammalian expression vector under control of a strong promoter. Cotransfection with cDNA encoding a fluorescent marker protein, such as Green Fluorescent Protein (CLONTECH), is useful for identifying stable transfectants. The amount of cell agglutination, or clumping, associated with transfected cells is compared with that associated with untransfected cells. The amount of cell agglutination is a direct measure of IRAP activity.

Various modifications and variations of the described compositions, methods, and systems of the invention will be apparent to those skilled in the art without departing from the scope and spirit of the invention. It will be appreciated that the invention provides novel and useful proteins, and their encoding polynucleotides, which can be used in the drug discovery process, as well as methods for using these compositions for the detection, diagnosis, and treatment of diseases and conditions. Although the invention has been described in connection with certain embodiments, it should be understood that the invention as claimed should not be unduly limited to such specific embodiments. Nor should the description of such embodiments be considered exhaustive or limit the invention to the precise forms disclosed. Furthermore, elements from one embodiment can be readily recombined with elements from one or more other embodiments. Such combinations can form a number of embodiments within the scope of the invention. It is intended that the scope of the invention be defined by the following claims and their equivalents.

TABLE 1

| Incyte | Polypeptide | Incyte | | Incyte | |
|---|---|---|---|---|---|
| Project | SEQ ID | Polypeptide | Polynucleotide | Polynucleotide | Incyte Full |
| ID | NO: | ID | SEQ ID NO: | ID | Length Clones |
| 7514650 | 1 | 7514650CD1 | 2 | 7514650CB1 | 90208684CA2 |

TABLE 2

| | | GenBank ID | | |
|---|---|---|---|---|
| | Incyte | NO: or | | |
| Polypeptide | Polypeptide | PROTEOME | Probability | |
| SEQ ID NO: | ID | ID NO: | Score | Annotation |
| 1 | 7514650CD1 | g186352 | 3.1E−80 | [Homo sapiens] interleukin 6 |
| | | | | Wong, G. G. et al. (1988) Interleukin 6: identification as a |
| | | | | hematopoietic colony-stimulating factor. Behring Inst. Mitt. |
| | | | | 83: 40-47. |
| | | 618376\|IL6 | 2.4E−81 | [Homo sapiens] [Ligand] [Cytoplasmic; Extracellular |
| | | | | (excluding cell wall)] Interleukin 6 (interferon-beta 2), |
| | | | | cytokine that promotes the proliferation of T and B cells and |
| | | | | final maturation of B-cells; implicated in multiple sclerosis and |
| | | | | the persistence of multiple myeloma and B-chronic lympohcytic |
| | | | | leukemia |
| | | | | Tosato, G. et al. (1988) Monocyte-derived human B-cell growth |
| | | | | factor identified as interferon- beta 2 (BSF-2, IL-6). Science |
| | | | | 239: 502-504. |
| | | | | Hirano, T. et al. (1986) Complementary DNA for a novel human |
| | | | | interleukin (BSF-2) that induces B lymphocytes to produce |
| | | | | immunoglobulin. Nature 324: 73-76. |
| | | | | Zilberstein, A. et al. (1986) Structure and expression of cDNA |
| | | | | and genes for human interferon-beta-2, a distinct species |
| | | | | inducible by growth-stimulatory cytokines. EMBO J 5: 2529-2537. |
| | | 590819\|I16 | 9.2E−29 | [Rattus norvegicus] [Ligand] [Extracellular (excluding |
| | | | | cell wall)] Interleukin 6, inflammatory cytokine that likely |
| | | | | promotes the proliferation and differentiation of B and T cells; |
| | | | | human IL6 is implicated in multiple sclerosis and the persistence of |
| | | | | multiple myeloma and chronic lymphocytic leukemia |
| | | | | Chiu, C. P. et al. (1988) Multiple biological activities are |
| | | | | expressed by a mouse interleukin 6 cDNA clone isolated from |
| | | | | bone marrow stromal cells. Proc. Natl. Acad. Sci. USA 85: |
| | | | | 7099-7103. |
| | | | | Northemann, W. et al. (1989) Structure of the rat interleukin |
| | | | | 6 gene and its expression in macrophage-derived cells. J. Biol. |
| | | | | Chem. 264: 16072-16082. |

TABLE 3

| SEQ | Incyte | Amino | | |
|---|---|---|---|---|
| ID | Polypeptide | Acid | | Analytical Methods |
| NO: | ID | Residues | Signature Sequences, Domains and Motifs | and Databases |
| 1 | 7514650CD1 | 163 | Signal_cleavage: M1-P27 | SPSCAN |
| | | | Signal Peptide: M1-V21, M1-A25, M1-F26, M1-P27 | HMMER |
| | | | Interleukin-6 homologues: I57-R161 | HMMER_SMART |
| | | | Interleukin-6/G-CSF/MGF family: I57-R161 | HMMER_PFAM |
| | | | Interleukin-6/G-CSF/MGF family | BLIMPS_BLOCKS |
| | | | IPB001716: L16-P27, E87-Q131, Q131-R161 | |
| | | | Interleukin-6/G-CSF/MGF family signature: | PROFILESCAN |
| | | | S80-R147 | |
| | | | Interleukin-6/G-CSF/MGF family signature | BLIMPS_PRINTS |
| | | | PR00433: Q56-T71, C72-M95, C101-L126, I145-R161 | |
| | | | Interleukin-6 signature | BLIMPS_PRINTS |
| | | | PR00434: Q56-C72, C78-G100, C101-T122, L144-R161 | |
| | | | FACTOR GROWTH GLYCOPROTEIN CYTOKINE PRECURSOR | BLAST_PRODOM |
| | | | SIGNAL INTERLEUKIN-6 | |
| | | | IL-6 GRANULOCYTE COLONY STIMULATING | |
| | | | PD004356: I57-I115 | |
| | | | INTERLEUKIN-6 PRECURSOR IL-6 CYTOKINE | BLAST_PRODOM |
| | | | GLYCOPROTEIN GROWTH FACTOR | |
| | | | SIGNAL B-CELL STIMULATORY | |
| | | | PD006297: M1-K55 | |
| | | | INTERLEUKIN-6/G-CSF/MGF | BLAST_DOMO |
| | | | DM02670\|P46650\|1-211: M1-T117, E108-M163 | |
| | | | DM02670\|P41323\|1-206: M1-T117, N107-M163 | |
| | | | DM02670\|P41683\|1-207: M1-T117, E108-M163 | |
| | | | DM02670\|P26892\|1-207: M1-A109, K110-R161 | |
| | | | Potential Phosphorylation Sites: S50 S80 S148 | MOTIFS |
| | | | S156 T48 T71 T116 | |
| | | | Potential Glycosylation Sites: N73 N123 | MOTIFS |

TABLE 4

| Polynucleotide | |
|---|---|
| SEQ ID NO:/ | |
| Incyte ID/ | |
| Sequence Length | Sequence Fragments |
| 2/7514650CB1/962 | 1-545, 2-962, 356-923, 737-951 |

TABLE 5

| Polynucleotide | | |
|---|---|---|
| SEQ ID NO: | Incyte Project ID: | Representative Library |
| 2 | 7514650CB1 | MPHGLPT02 |

TABLE 6

| Library | Vector | Library Description |
|---|---|---|
| MPHGLPT02 | PSPORT1 | Library was constructed using RNA |
| | | isolated from adherent mononuclear |
| | | cells, which came from a pool of male |
| | | and female donors. The cells were |
| | | stimulated with LPS. |

TABLE 7

| Program | Description | Reference | Parameter Threshold |
|---|---|---|---|
| ABI FACTURA | A program that removes vector | Applied Biosystems, Foster | |
| | sequences and masks ambiguous | City, CA. | |
| | bases in nucleic acid sequences. | | |
| ABI/PARACEL | A Fast Data Finder useful in | Applied Biosystems, Foster | Mismatch <50% |
| FDF | comparing and annotating amino | City, CA; Paracel Inc., | |
| | acid or nucleic acid sequences. | Pasadena, CA. | |
| ABI | A program that assembles nucleic | Applied Biosystems, Foster | |
| AutoAssembler | acid sequences. | City, CA. | |
| BLAST | A Basic Local Alignment Search | Altschul, S. F. et al. (1990) | ESTs: Probability value = |
| | Tool useful in sequence similarity | J. Mol. Biol. 215: 403-410; | 1.0E−8 or less; Full Length |
| | search for amino acid and nucleic | Altschul, S. F. et al. (1997) | sequences: Probability value = |
| | acid sequences. BLAST includes | Nucleic Acids Res. 25: 3389- | 1.0E−10 or less |
| | five functions: blastp, blastn, | 3402. | |
| | blastx, tblastn, and tblastx. | | |
| FASTA | A Pearson and Lipman algorithm that | Pearson, W. R. and D. J. Lipman | ESTs: fasta E value = |
| | searches for similarity between a | (1988) Proc. Natl. Acad Sci. USA | 1.06E−6; Assembled ESTs: |
| | query sequence and a group of | 85: 2444-2448; Pearson, W. R. | fasta Identity = 95% or |
| | sequences of the same type. FASTA | (1990) Methods Enzymol. 183: 63-98; | greater and Match length = |
| | comprises as least five functions: | and Smith, T. F. and M. S. Waterman | 200 bases or greater; fastx E |
| | fasta, tfasta, fastx, tfastx, and | (1981) Adv. Appl. Math. 2: 482-489. | value = 1.0E−8 or less; |
| | ssearch. | | Full Length sequences: fastx |
| | | | score = 100 or greater |
| BLIMPS | A BLocks IMProved Searcher that | Henikoff, S. and J. G. Henikoff | Probability value = |
| | matches a sequence against those in | (1991) Nucleic Acids Res. 19: | 1.0E−3 or less |
| | BLOCKS, PRINTS, DOMO, PRODOM, and | 6565-6572; Henikoff, J. G. and S. | |
| | PFAM databases to search for gene | Henikoff (1996) Methods Enzymol. | |
| | families, sequence homology, and | 266: 88-105; and Attwood, T. K. | |
| | structural fingerprint regions. | et al. (1997) J. Chem. Inf. | |
| | | Comput. Sci. 37: 417-424. | |
| HMMER | An algorithm for searching a query | Krogh, A. et al. (1994) J. Mol. | PFAM, INCY, SMART or TIGRFAM |
| | sequence against hidden Markov model | Biol. 235: 1501-1531; Sonnhammer, | hits: Probability value = |
| | (HMM)-based databases of protein family | E. L. L. et al. (1988) Nucleic | 1.0E−3 or less; Signal |
| | consensus sequences, such as PFAM, | Acids Res. 26: 320-322; Durbin, R. | peptide hits: Score = 0 |
| | INCY, SMART and TIGRFAM. | et al. (1998) Our World View, in a | or greater |
| | | Nutshell, Cambridge Univ. Press, | |
| | | pp. 1-350. | |
| ProfileScan | An algorithm that searches for | Gribskov, M. et al. (1988) CABIOS | Normalized quality score ≧ |
| | structural and sequence motifs | 4: 61-66; Gribskov, M. et al. (1989) | GCG-specified “HIGH” |
| | in protein sequences that match | Methods Enzymol. 183: 146-159; | value for that particular |
| | sequence patterns defined in Prosite. | Bairoch, A. et al. (1997) Nucleic | Prosite motif. Generally, |
| | | Acids Res. 25: 217-221. | score = 1.4-2.1. |
| Phred | A base-calling algorithm that examines | Ewing, B. et al. (1998) Genome | |
| | automated sequencer traces with high | Res. 8: 175-185; Ewing, B. and | |
| | sensitivity and probability. | P. Green (1998) Genome Res. 8: | |
| | | 186-194. | |
| Phrap | A Phils Revised Assembly Program | Smith, T. F. and M. S. Waterman | Score = 120 or greater; |
| | including SWAT and CrossMatch, programs | (1981) Adv. Appl. Math. 2: 482- | Match length = 56 or |
| | based on efficient implementation | 489; Smith, T. F. and M. S. | greater |
| | of the Smith-Waterman algorithm, useful | Waterman (1981) J. Mol. Biol. | |
| | in searching sequence homology and | 147: 195-197; and Green, P., | |
| | assembling DNA sequences. | University of Washington, | |
| | | Seattle, WA. | |
| Consed | A graphical tool for viewing and | Gordon, D. et al. (1998) Genome | |
| | editing Phrap assemblies. | Res. 8: 195-202. | |
| SPScan | A weight matrix analysis program that | Nielson, H. et al. (1997) Protein | Score = 3.5 or greater |
| | scans protein sequences for the presence | Engineering 10: 1-6; Claverie, | |
| | of secretory signal peptides. | J. M. and S. Audic (1997) CABIOS | |
| | | 12: 431-439. | |
| TMAP | A program that uses weight matrices | Persson, B. and P. Argos (1994) | |
| | to delineate transmembrane segments | J. Mol. Biol. 237: 182-192; | |
| | on protein sequences and determine | Persson, B. and P. Argos (1996) | |
| | orientation. | Protein Sci. 5: 363-371. | |
| TMHMMER | A program that uses a hidden Markov | Sonnhammer, E. L. et al. (1998) | |
| | model (HMM) to delineate transmembrane | Proc. Sixth Intl. Conf. On | |
| | segments on protein sequences | Intelligent Systems for Mol. | |
| | and determine orientation. | Biol., Glasgow et al., eds., | |
| | | The Am. Assoc. for Artificial | |
| | | Intelligence (AAAI) Press, Menlo | |
| | | Park, CA, and MIT Press, Cambridge, | |
| | | MA, pp. 175-182. | |
| Motifs | A program that searches amino acid | Bairoch, A. et al. (1997) Nucleic | |
| | sequences for patterns that matched | Acids Res. 25: 217-221; Wisconsin | |
| | those defined in Prosite. | Package Program Manual, version 9, | |
| | | page M51-59, Genetics Computer | |
| | | Group, Madison, WI. | |

TABLE 8

| SEQ | | | | | | EST | Al- | Al- | | Caucasian | African | Asian | Hispanic |
|---|---|---|---|---|---|---|---|---|---|---|---|---|---|
| ID | | | | EST | CB1 | Al- | lele | lele | Amino | Allele 1 | Allele 1 | Allele 1 | Allele 1 |
| NO: | PID | EST ID | SNP ID | SNP | SNP | lele | 1 | 2 | Acid | frequency | frequency | frequency | frequency |
| 2 | 7514650 | 2408395H1 | SNP00026991 | 85 | 458 | C | C | A | T142 | n/a | n/a | n/a | n/a |
| 2 | 7514650 | 2482074F6 | SNP00042027 | 348 | 337 | T | T | C | F102 | n/a | n/a | n/a | n/a |

What is claimed is:

1-125. (canceled)

126. An isolated polypeptide selected from the group consisting of:
(a) a polypeptide comprising an amino acid sequence of SEQ ID NO: 1;
(b) a biologically active fragment of the polypeptide of (a); and
(c) an immunogenic fragment of the polypeptide of (a).

127. An isolated polypeptide of claim 126 consisting of the polypeptide of (a).

128. An isolated polypeptide of claim 126 consisting of a biologically active fragment of the polypeptide of (a).

129. An isolated polypeptide of claim 126 consisting of an immunogenic fragment of the polypeptide of (a).

130. An isolated polypeptide of claim 126 encoded by a polynucleotide selected from the group consisting of:
(i) a polynucleotide comprising a polynucleotide sequence of SEQ ID NO: 2;
(ii) a polynucleotide comprising a naturally occurring polynucleotide sequence at least 90% identical to SEQ ID NO: 2;
(iii) a polynucleotide comprising a portion of the polynucleotide sequence of SEQ ID NO: 2 that specifically identifies SEQ ID NO: 2.
(iv) a polynucleotide comprising a polynucleotide complementary to the polynucleotide of (i), (ii), or (iii);
(v) an RNA equivalent of the polynucleotide of (i), (ii), (iii) or (iv);
(vi) a polynucleotide of (i), (ii) or (iii) further comprising a promoter sequence operably linked to said polynucleotide of (i), (ii) or (iii).

131. An isolated polypeptide of claim 126 produced recombinantly.

132. An isolated polypeptide of claim 130 produced by culturing a cell transformed with a polynucleotide of (iv) under conditions suitable for expression of the polypeptide, and recovering the polypeptide so expressed.

133. An isolated antibody that specifically binds to a polypeptide of claim 126.

134. An isolated antibody of claim 133, wherein said antibody is selected from the group consisting of a polyclonal antibody, a monoclonal antibody, a chimeric antibody, a single chain antibody, a Fab fragment, a F(ab′)₂ fragment, and a humanized antibody.

135. An isolated antibody of claim 134, wherein said antibody is selected by screening a recombinant immunoglobulin library.

136. An isolated antibody of claim 134, wherein said antibody is selected by screening a Fab expression library.

137. An isolated antibody that specifically binds to a polypeptide of claim 130.

138. An isolated antibody of claim 137, wherein said antibody is selected from the group consisting of a polyclonal antibody, a monoclonal antibody, a chimeric antibody, a single chain antibody, a Fab fragment, a F(ab′)₂ fragment, and a humanized antibody.

139. An isolated antibody of claim 137, wherein said antibody is selected by screening a recombinant immunoglobulin library.

140. An isolated antibody of claim 137, wherein said antibody is selected by screening a Fab expression library.

141. A method of detecting a polypeptide of interest in a sample, comprising:
incubating the sample with an antibody that specifically binds to a polypeptide of claim 126 under conditions suitable for binding of the antibody to the polypeptide of interest if present in the sample; and
detecting biding of the polypeptide of interest to the antibody, wherein binding indicates the presence or amount of the polypeptide of interest in the sample.

142. A method of claim 141, wherein the sample is a body fluid sample from a human.

143. An isolated polynucleotide selected from the group consisting of:
(i) a polynucleotide comprising a polynucleotide sequence of SEQ ID NO: 2;
(ii) a polynucleotide comprising a naturally occurring polynucleotide sequence at least 90% identical to SEQ ID NO: 2;
(iii) a polynucleotide comprising a portion of the polynucleotide sequence of SEQ ID NO: 2 that specifically identifies SEQ ID NO: 2.
(iv) a polynucleotide comprising a polynucleotide complementary to the polynucleotide of (i), (ii), or (iii);
(v) an RNA equivalent of the polynucleotide of (i), (ii), (iii) or (iv);
(vi) a polynucleotide of (i), (ii) or (iii) further comprising a promoter sequence operably linked to said polynucleotide of (i), (ii) or (iii).
